(19) United States
(12) Patent Application Publication
(10) Pub. No.: US 2026/0115008 A1
(43) Pub. Date: Apr. 30, 2026

(54) HIP NAVIGATION SYSTEMS AND METHODS

(71) Applicant: OrthAlign, Inc., Irvine, CA (US)

(72) Inventors: James Gannoe, Laguna Niguel, CA (US); Scott Seidman, Culver City, CA (US); Steven DeVincentis, Laguna Niguel, CA (US); Sean Cantarini, Laguna Niguel, CA (US)

(21) Appl. No.: 19/339,925

(22) Filed: Sep. 25, 2025

Related U.S. Application Data
(60) Provisional application No. 63/704,778, filed on Oct. 8, 2024.

(51) Int. Cl. A61F 2/46; A61F 2/30; A61F 2/36

(52) U.S. Cl. CPC A61F 2/4609; A61F 2/36; A61F 2/4684; A61F 2002/30538; A61F 2002/30617; A61F 2002/4687; A61F 2250/0096

(57) ABSTRACT

A hip navigation system is provided that includes a navigation device comprising one or more sensors configured to sense changes in orientation and rotation, a reference sensor device comprising one or more sensors configured to sense changes in orientation and rotation, and a femur sensor device comprising one or more sensors configured to sense changes in orientation and rotation. The hip navigation system is provided for navigating a cup toward target angles. The hip navigation system is provided for determining changes in leg length and/or joint offset. Methods of using the hip navigation system are provided. A graphical user interface of the navigation device is provided.

### INCORPORATION BY REFERENCE TO ANY PRIORITY APPLICATIONS

This application claims the benefit under 35 U.S.C. § 119(e) to U.S. Provisional Application No. 63/704,778, filed Oct. 8, 2024, which is hereby incorporated by reference in its entirety. Any and all applications for which a foreign or domestic priority claim is identified in the Application Data Sheet as filed with the present application is hereby incorporated by reference in its entirety under 37 CFR 1.57.

### BACKGROUND

### Field

This application is directed to the field of hip replacement, and particularly to surgical tools and methods for guiding the preparation of the bones in connection therewith.

### Description of the Related Art

Hip replacement surgery is common and getting more common by the year. One persistent issue with hip replacement is the relatively high incidence of poor placement of the cup and ball components of the prosthetic hip joint. For example, the cup is optimally placed in a specific alignment with a plane including a rim of the acetabulum of the pelvis. For several reasons an unacceptably high percentage of patients have the cup of the artificial hip joint out of alignment with this plane.

Unfortunately, misalignment can lead to dislocation of the hip as soon as within one year of the implantation procedure. This is particularly problematic because recovery from a hip procedure can take many months. Patients undergoing a revision so soon after the initial implantation will certainly be dissatisfied with their care, being subject to addition redundant surgery. Of course, all surgery carries some degree of risk. These poor outcomes are unsatisfactory for patients and surgeons and are inefficient for the healthcare system as a whole.

Also, in cup placement in total hip arthroplasty, the abduction and anteversion angles are with respect to the Anterior Pelvic Plane (defined as a plane created by the two anterior superior iliac spines (ASIS) and the pubic symphysis). While these anatomical features are visible/palpable while the patient is in a supine position, the majority of total hip replacements are accomplished via a posterolateral approach with the patient in some variation of a lateral position, in which most of these landmarks are not accessible or visible. Historically, navigation for posterior approach hip replacement has been accomplished using equipment linked to a computer to register the anatomical features of the Anterior Pelvic Plane with the patient first in a supine position and, once this plane is recorded by the navigation computer, moving the patient to a lateral position in order to perform hip surgery-with navigation performed with respect to the directly registered Anterior Pelvic Plane. This approach to hip navigation is sub-optimal for surgical workflow because the extra movement of the patient from supine to lateral position takes more surgeon and staff time and requires breaking sterility and re-draping. This is one of the key reasons why hip navigation has failed to be adopted by most of the market.

Additionally, altered leg length is a common patient complaint arising from hip replacement surgery and has been a common cause of patient dissatisfaction and complaints that arise from hip replacement. Because part of the hip replacement procedure requires precise measurements of patient leg length and joint off-set that are frequently difficult to visualize utilizing conventional instrumentation, there are opportunities to improve the surgeon's performance of these measurements using computer technology.

### SUMMARY OF THE INVENTION

There is a need for improved systems and methods for providing for proper placement, e.g., alignment, of hip components with a patient's anatomy during a hip replacement procedure. This can involve techniques for registering a reference frame. This can involve techniques for confirming alignment of a prosthetic component with target angles. This can involve techniques for measuring leg length and joint offset.

In some embodiments, a method of performing a hip joint replacement procedure on a patient in provided. The hip joint comprises a pelvis and a femur that is movable relative to the pelvis. The method can include positioning the femur relative to the pelvis. The method can include registering a femur registration marker. The method can include registering a point of a femur registration marker. The method can include inserting a cup and a liner into an acetabulum of the pelvis. The method can include inserting a concave structure into an acetabulum of the pelvis. The method can include registering a center of rotation by inserting a liner registration trial into the liner. The method can include inserting a liner registration trial into the liner. The method can include thereafter registering a center of rotation. The method can include repositioning the femur relative to the pelvis after inserting the cup. The method can include registering the femur registration marker after inserting the cup. The method can include registering the point of the femur registration marker after inserting the cup.

In some embodiments, the method can include coupling the liner registration trial to a registration wand. In some embodiments, registering the center of rotation comprises registering a point on the registration wand. In some embodiments, the method can include positioning a femur sensor device on a distal thigh of the patient while registering the point of the femur registration marker. In some embodiments, the method can include positioning a femur sensor device on the greater trochanter while registering the point of the femur registration marker. In some embodiments, registering the point of the femur registration marker comprises coupling a reference sensor device to a registration probe. In some embodiments, registering the center of rotation comprises coupling a reference sensor device to a registration probe.

In some embodiments, a system for orthopedic surgery is provided. The system can include a pelvic bracket configured to couple to a bone of a patient, wherein the pelvic bracket comprises a first interface, a second interface, and a third interface. The system can include a navigation device configured to couple to the first interface. In some embodiments, the navigation device comprises one or more sensors configured to sense changes in orientation and rotation. The system can include a reference sensor device configured to couple to the second interface. In some embodiments, the reference sensor device comprises one or more sensors configured to sense changes in orientation and rotation. The system can include a femur sensor device configured to couple to the third interface. In some embodiments, the femur sensor device comprises one or more sensors configured to sense changes in orientation and rotation.

In some embodiments, a system for orthopedic surgery is provided. The system can include a bracket configured to couple to a bone of a patient, wherein the bracket comprises a first interface, a second interface, and a third interface. The system can include a navigation device configured to couple to the first interface. In some embodiments, the navigation device comprises one or more sensors configured to sense changes in orientation and rotation. The system can include a reference sensor device configured to couple to the second interface. In some embodiments, the reference sensor device comprises one or more sensors configured to sense changes in orientation and rotation. The system can include a bone sensor device configured to couple to the third interface. In some embodiments, the bone sensor device comprises one or more sensors configured to sense changes in orientation and rotation.

In some embodiments, at least one of the navigation device, the reference sensor device, and/or the femur sensor device is configured to communicate changes in orientation and rotation of the at least one of the navigation device, the reference sensor device, and/or the femur sensor device to another of the at least one of the navigation device, the reference sensor device, and/or the femur sensor device. In some embodiments, at least one of the navigation device, the reference sensor device, and/or the femur sensor device is configured to calculate leg length. In some embodiments, at least one of the navigation device, the reference sensor device, and/or the femur sensor device is configured to calculate joint offset. In some embodiments, at least one of the navigation device, the reference sensor device, and/or the femur sensor device is configured to calculate a center of rotation. In some embodiments, at least one of the navigation device, the reference sensor device, and/or the femur sensor device is configured to calculate a leg length direction. In some embodiments, the navigation device is configured to receive an input related to pelvic tilt. In some embodiments, the navigation device is configured to receive an input related a patient specific reference frame. In some embodiments, the system can include a processor. In some embodiments, the processor is configured to mathematically rotate a femur about a center of rotation.

In some embodiments, a system for orthopedic surgery is provided.. The system can include a thigh support configured to couple to a distal thigh of a patient. The system can include a thigh a plate configured to couple to a distal thigh of a patient. The system can include a thigh mount configured to couple to the thigh support. The system can include a thigh mount configured to couple to the thigh plate. The system can include a femur sensor device configured to couple to the thigh mount. In some embodiments, the femur sensor device comprises one or more sensors configured to sense changes in orientation and rotation.

In some embodiments, the system can include a pelvic bracket. In some embodiments, the femur sensor device is configured to couple to the pelvic bracket. In some embodiments, the system can include a registration wand. In some embodiments, the femur sensor device is configured to couple to the registration wand. In some embodiments, the system can include an impactor adapter. In some embodiments, the femur sensor device is configured to couple to the impactor adapter. In some embodiments, the system can include an impactor. In some embodiments, the impactor adapter is configured to couple to the impactor.

In some embodiments, a system for orthopedic surgery is provided. The system can include a pelvic bracket configured to couple to a bone of a patient. In some embodiments, the pelvic bracket comprises a first interface and a second interface. The system can include a first sensor device configured to couple to the first interface. In some embodiments, the first device comprises one or more sensors configured to sense changes in orientation and rotation. The system can include a second sensor device configured to couple to the second interface. In some embodiments, the second sensor device comprises one or more sensors configured to sense changes in orientation and rotation. In some embodiments the second sensor device is configured to be transferred from the second interface to a location on a femur of a patient.

In some embodiments, the second sensor device is configured to communicate changes in orientation and rotation of the second sensor device to the first sensor device. In some embodiments, the first sensor device is configured to calculate leg length. In some embodiments, the first sensor device is configured to calculate joint offset. In some embodiments, the first sensor device is configured to calculate a center of rotation. In some embodiments, the first sensor device is configured to calculate a leg length direction. In some embodiments, the first sensor device is configured to receive an input related to pelvic tilt. In some embodiments, the first sensor device is configured to receive an input related a patient specific reference frame. In some embodiments, the system can include a processor. In some embodiments, the processor is configured to mathematically rotate a femur about a center of rotation. In some embodiments, the system can include a thigh mount. In some embodiments, the thigh mount is configured to be positioned at the location on the femur of the patient. In some embodiments, the second sensor device is configured to sense changes in orientation and rotation of the femur. In some embodiments, the first sensor device is configured to calculate a change in rotation of the femur.

In some embodiments, a system for orthopedic surgery is provided. The system can include a femur sensor device. In some embodiments, the femur sensor device comprises one or more sensors configured to sense changes in orientation and rotation. The system can include a registration wand. In some embodiments, the femur sensor device is configured to couple to the registration wand.

In some embodiments, the system can include a plurality of liner registration trials. In some embodiments, each liner registration trial is configured to couple to the registration wand. In some embodiments, the system can include a liner registration trial configured to couple to the registration wand. In some embodiments, the liner registration trial is configured to be inserted into a liner of a cup. In some embodiments, the registration wand comprises a divot. In some embodiments, a tip of a registration probe is configured to contact the divot to register the registration wand.

### BRIEF DESCRIPTION OF THE DRAWINGS

These and other features, aspects and advantages are described below with reference to the drawings, which are intended to illustrate but not to limit the inventions. In the drawings, like reference characters denote corresponding features consistently throughout similar embodiments.

FIG. 1 is a perspective view of a hip navigation system applied to a patient illustrating registering a point on a femur in connection with a measurement of leg length and/or joint offset before or after implantation of the prosthetic hip joint.

FIG. 2 is a front view of a navigation device of the hip navigation system FIG. 1.

FIG. 3 is a perspective view of a reference sensor device of the hip navigation system FIG. 1.

FIG. 4 is a perspective view of a femur sensor device of the hip navigation system FIG. 1.

FIG. 5 is a view of a screen of a graphical user interface that can be displayed on a component of the hip navigation system of FIG. 1, e.g., on the navigation device of FIG. 2.

FIG. 6 is a view of a screen of the graphical user interface that can be displayed on a component of the hip navigation system of FIG. 1.

FIG. 7 is a view of a screen of the graphical user interface that can be displayed on a component of the hip navigation system of FIG. 1.

FIG. 8 is a view of a screen of the graphical user interface that can be displayed on a component of the hip navigation system of FIG. 1.

FIG. 9 is a view of the components of one embodiment of a kit including the hip navigation system of FIG. 1.

FIG. 10 is a view of packaging of the navigation device.

FIG. 11 is a view of packaging of the navigation device.

FIG. 12 is a view of the navigation device.

FIG. 13 is a view of the navigation device.

FIG. 14 is a view of the navigation device and the reference sensor device.

FIG. 15 is a view of a screen of the graphical user interface that can be displayed on a component of the hip navigation system of FIG. 1.

FIG. 16 is a view of the reference sensor device.

FIG. 17 is a view of the reference sensor device.

FIG. 18 is a view of a screen of the graphical user interface that can be displayed on a component of the hip navigation system of FIG. 1.

FIG. 19 is a view of a screen of the graphical user interface that can be displayed on a component of the hip navigation system of FIG. 1.

FIG. 20 is a view of a screen of the graphical user interface that can be displayed on a component of the hip navigation system of FIG. 1.

FIG. 21 is a view of a screen of the graphical user interface that can be displayed on a component of the hip navigation system of FIG. 1.

FIG. 22 is a view of a screen of the graphical user interface that can be displayed on a component of the hip navigation system of FIG. 1.

FIG. 23 is a view of a screen of the graphical user interface that can be displayed on a component of the hip navigation system of FIG. 1.

FIG. 24 is a view of a screen of the graphical user interface that can be displayed on a component of the hip navigation system of FIG. 1.

FIG. 25 is a view of a screen of the graphical user interface that can be displayed on a component of the hip navigation system of FIG. 1.

FIG. 26 is a view of a screen of the graphical user interface that can be displayed on a component of the hip navigation system of FIG. 1.

FIG. 27 is a view of the navigation device disposed adjacent to a pelvic bracket to be coupled thereto.

FIG. 28 is a view of the navigation device coupled to the pelvic bracket and the reference sensor device disposed adjacent to the pelvic bracket to be coupled thereto.

FIG. 29 is a view of the reference sensor device coupled to the pelvic bracket and the femur sensor device adjacent to the pelvic bracket to be couple thereto.

FIG. 30 is a view of an assembly during a calibration step including the navigation device, the reference sensor device, and the femur sensor device coupled to the pelvic bracket.

FIG. 31 is a view of the assembly during a calibration step including the navigation device, the reference sensor device, and the femur sensor device coupled to the pelvic bracket.

FIG. 32 is a view of the assembly during a calibration step including the navigation device, the reference sensor device, and the femur sensor device coupled to the pelvic bracket.

FIG. 33 is a view of the assembly during a calibration step including the navigation device, the reference sensor device, and the femur sensor device coupled to the pelvic bracket.

FIG. 34 is a view of the reference sensor device disposed adjacent to a registration probe to be coupled thereto.

FIG. 35 is a view of the assembly including the navigation device, the registration probe, and the femur sensor device coupled to the pelvic bracket disposed adjacent to a probe calibration jig to be coupled thereto.

FIG. 36 is a view of the navigation device, the reference sensor device, the femur sensor device, and the probe calibration jig in a first calibration position.

FIG. 37 is a view of the navigation device, the reference sensor, the femur sensor device, and the probe calibration jig in a second calibration position.

FIG. 38 is a view of the navigation device, the reference sensor device, the femur sensor device, and the probe calibration jig in a third calibration position.

FIG. 39 is an exploded view of the navigation device and the pelvic bracket from a side position.

FIG. 40 is a view of the navigation device and the femur sensor coupled to the pelvic bracket.

FIG. 41 is a view of the navigation device, the reference sensor device, and the femur sensor device coupled to the pelvic bracket.

FIG. 42 is a view of an impactor adapter.

FIG. 43 is a view of the impactor adapter and an impactor.

FIG. 44 is a view of a femur registration marker.

FIG. 45 is a view of a thigh plate.

FIG. 46 is a view of a thigh mount.

FIG. 47 is a view of a fixation pin of the hip navigation system FIG. 1 disposed adjacent to the pelvis before insertion.

FIG. 48 is a view of fixation pins of the hip navigation system FIG. 1 inserted into the pelvis.

FIG. 49 is a view of a pelvic base of the hip navigation system FIG. 1 coupled to the fixation pins inserted into the pelvis.

FIG. 50 is a view of the navigation device, the reference sensor device, the femur sensor device, a pelvic bracket, and a pelvic base coupled to the fixation pins inserted into the pelvis.

FIG. 51 is a view of the thigh mount coupled to the distal thigh and the femur registration marker coupled to the greater trochanter.

FIG. 52 is a view of the femur registration marker.

FIG. 53 is a view of a fixation tower and the femur sensor device coupled to the fixation tower.

FIG. 54 is a view of a screen of the graphical user interface that can be displayed on a component of the hip navigation system of FIG. 1.

FIG. 55 is a view of a screen of the graphical user interface that can be displayed on a component of the hip navigation system of FIG. 1.

FIG. 56 is a view of the navigation device, the reference sensor device, the femur sensor device, and the registration probe.

FIG. 57 is a view of a screen of a graphical user interface that can be displayed on a component of the hip navigation system of FIG. 1.

FIG. 58 is a view of the femur registration marker coupled to the greater trochanter.

FIG. 59 is a view of the femur sensor device coupled to the pelvic bracket.

FIG. 60 is a view of the navigation device, the reference sensor device, the femur sensor device, and the registration probe during registration of a point.

FIG. 61 is a view of the femur sensor device coupled to the thigh mount.

FIG. 62 is a view of the femur registration marker coupled to a fixation plate.

FIG. 63 is a view of the navigation device, the reference sensor device, the femur sensor device, and the registration probe during registration of a point.

FIG. 64 is a view of the femur sensor device coupled to the fixation tower.

FIG. 65 is a view of the navigation device, the reference sensor device, the femur sensor device, and the registration probe during registration of a point.

FIG. 66 is a view of the femur sensor device coupled to the fixation tower.

FIG. 67 is a view of the impactor adapter and the impactor.

FIG. 68 is a view of the navigation device and the femur sensor device coupled to the pelvic bracket.

FIG. 69 is a view of the femur sensor device disposed adjacent to the impactor adapter and the impactor to be coupled thereto.

FIG. 70 is a view of a screen of the graphical user interface that can be displayed on a component of the hip navigation system of FIG. 1.

FIG. 71 is a view of a screen of the graphical user interface that can be displayed on a component of the hip navigation system of FIG. 1.

FIG. 72 is a view of a screen of the graphical user interface that can be displayed on a component of the hip navigation system of FIG. 1.

FIG. 73 is a view of the femur sensor device, the impactor adapter, and the impactor for cup placement.

FIG. 74 is a view of a screen of the graphical user interface that can be displayed on a component of the hip navigation system of FIG. 1.

FIG. 75 is a view of a screen of the graphical user interface that can be displayed on a component of the hip navigation system of FIG. 1.

FIG. 76 is a view of a cup and a liner disposed adjacent to an acetabulum for insertion therein.

FIG. 77 is a view of a registration wand and a plurality of liner registration trials in an embodiment of a kit.

FIG. 78 is a view of the registration wand and a liner registration trial disposed adjacent to the registration wand to be coupled thereto.

FIG. 79 is a view of the registration wand and the liner registration trial.

FIG. 80 is a view of a screen of the graphical user interface that can be displayed on a component of the hip navigation system of FIG. 1.

FIG. 81 is a view of a screen of the graphical user interface that can be displayed on a component of the hip navigation system of FIG. 1.

FIG. 82 is a view of a screen of the graphical user interface that can be displayed on a component of the hip navigation system of FIG. 1.

FIG. 83 is a view of a screen of the graphical user interface that can be displayed on a component of the hip navigation system of FIG. 1.

FIG. 84 is a view of the femur sensor device, the registration probe, and the registration wand for determining a center of rotation.

FIG. 85 is a view of the femur registration marker inserted into the greater trochanter.

FIG. 86 is a view of the femur sensor device coupled to the thigh mount.

FIG. 87 is a view of a screen of the graphical user interface that can be displayed on a component of the hip navigation system of FIG. 1.

FIG. 88 is a view of the navigation device, the reference sensor device, the femur sensor device, and the registration probe during registration of a point.

FIG. 89 is a view of the femur registration marker and the fixation plate.

FIG. 90 is a view of a screen of the graphical user interface that can be displayed on a component of the hip navigation system of FIG. 1.

FIG. 91 is a view of the femur sensor device.

FIG. 92 is a view of a screen of the graphical user interface that can be displayed on a component of the hip navigation system of FIG. 1.

FIG. 93 is a view of the navigation device, the reference sensor device, the femur sensor device, and the registration probe during registration of a point.

FIGS. 94A-94B are views of the femur sensor device.

FIGS. 95A-95B are views of the reference sensor device.

FIG. 96 is a view of the navigation device and the femur sensor device coupled to the pelvic bracket.

FIG. 97 is a view of the thigh plate and the thigh mount coupled to the distal thigh.

FIG. 98 is a view of a screen of the graphical user interface that can be displayed on a component of the hip navigation system of FIG. 1.

FIG. 99 is a view of a screen of the graphical user interface that can be displayed on a component of the hip navigation system of FIG. 1.

FIG. 100 is a view of a screen of the graphical user interface that can be displayed on a component of the hip navigation system of FIG. 1.

FIG. 101 is a view of a screen of the graphical user interface that can be displayed on a component of the hip navigation system of FIG. 1.

FIG. 102 is a view of a screen of the graphical user interface that can be displayed on a component of the hip navigation system of FIG. 1.

FIG. 103 is a view of a screen of the graphical user interface that can be displayed on a component of the hip navigation system of FIG. 1.

FIG. 104 is a view of a screen of the graphical user interface that can be displayed on a component of the hip navigation system of FIG. 1.

FIG. 105 is a view of a screen of the graphical user interface that can be displayed on a component of the hip navigation system of FIG. 1.

FIG. 106 is a view of a screen of the graphical user interface that can be displayed on a component of the hip navigation system of FIG. 1.

FIG. 107 is a view of a screen of the graphical user interface that can be displayed on a component of the hip navigation system of FIG. 1.

FIG. 108 is a view of a screen of the graphical user interface that can be displayed on a component of the hip navigation system of FIG. 1.

FIG. 109 is a view of a screen of the graphical user interface that can be displayed on a component of the hip navigation system of FIG. 1.

FIG. 110 is a view of a screen of the graphical user interface that can be displayed on a component of the hip navigation system of FIG. 1.

FIG. 111 is a view of a screen of the graphical user interface that can be displayed on a component of the hip navigation system of FIG. 1.

FIG. 112 is a view of a screen of the graphical user interface that can be displayed on a component of the hip navigation system of FIG. 1.

FIG. 113 is a view of a screen of the graphical user interface that can be displayed on a component of the hip navigation system of FIG. 1.

FIG. 114 is a view of a screen of the graphical user interface that can be displayed on a component of the hip navigation system of FIG. 1.

FIG. 115 is a view of a screen of the graphical user interface that can be displayed on a component of the hip navigation system of FIG. 1.

FIG. 116 is a view of a screen of the graphical user interface that can be displayed on a component of the hip navigation system of FIG. 1.

FIG. 117 is a view of a screen of the graphical user interface that can be displayed on a component of the hip navigation system of FIG. 1.

FIG. 118 is a view of a screen of the graphical user interface that can be displayed on a component of the hip navigation system of FIG. 1.

FIG. 119 is a view of a screen of the graphical user interface that can be displayed on a component of the hip navigation system of FIG. 1.

FIG. 120 is a view of a screen of the graphical user interface that can be displayed on a component of the hip navigation system of FIG. 1.

FIG. 121 is a view of a screen of the graphical user interface that can be displayed on a component of the hip navigation system of FIG. 1.

FIG. 122 is a view of a screen of the graphical user interface that can be displayed on a component of the hip navigation system of FIG. 1.

FIG. 123 is a view of a screen of the graphical user interface that can be displayed on a component of the hip navigation system of FIG. 1.

FIG. 124 is a view of a screen of the graphical user interface that can be displayed on a component of the hip navigation system of FIG. 1.

FIG. 125 is a view of a screen of the graphical user interface that can be displayed on a component of the hip navigation system of FIG. 1.

FIG. 126 is a view of a screen of the graphical user interface that can be displayed on a component of the hip navigation system of FIG. 1.

FIG. 127 is a view of a screen of the graphical user interface that can be displayed on a component of the hip navigation system of FIG. 1.

FIG. 128 is a view of a screen of the graphical user interface that can be displayed on a component of the hip navigation system of FIG. 1.

FIG. 129 is a view of a screen of the graphical user interface that can be displayed on a component of the hip navigation system of FIG. 1.

FIG. 130 is a view of a screen of the graphical user interface that can be displayed on a component of the hip navigation system of FIG. 1.

FIG. 131 is a view of a screen of the graphical user interface that can be displayed on a component of the hip navigation system of FIG. 1.

FIG. 132 is a view of a screen of the graphical user interface that can be displayed on a component of the hip navigation system of FIG. 1.

FIG. 133 is a view of a screen of the graphical user interface that can be displayed on a component of the hip navigation system of FIG. 1.

### DETAILED DESCRIPTION

A variety of systems and methods are discussed below that can be used to improve outcomes for patients by increasing the likelihood of proper placement prosthetic hip joint components for hip replacement. These systems can be focused on inertial navigation techniques.

### I. HIP Navigation Using Inertial Sensors

Systems and methods described below can improve prosthetic hip joint placement using navigation. These hip procedures generally guide a prosthetic cup to an orientation within the acetabulum that minimizes the chance of dislocation due to impingement of the femoral neck on the cup or on bones around the acetabulum or other reasons related to suboptimal orientation of the prosthetic cup. Various techniques leverage population averages of proper placement while others are amenable to patient specific refinements. Also various techniques for registering and confirming the position and/or orientation of the femur pre-and post-implantation of the cup are discussed herein, which are useful to control leg length and joint offset at the end of the procedure.

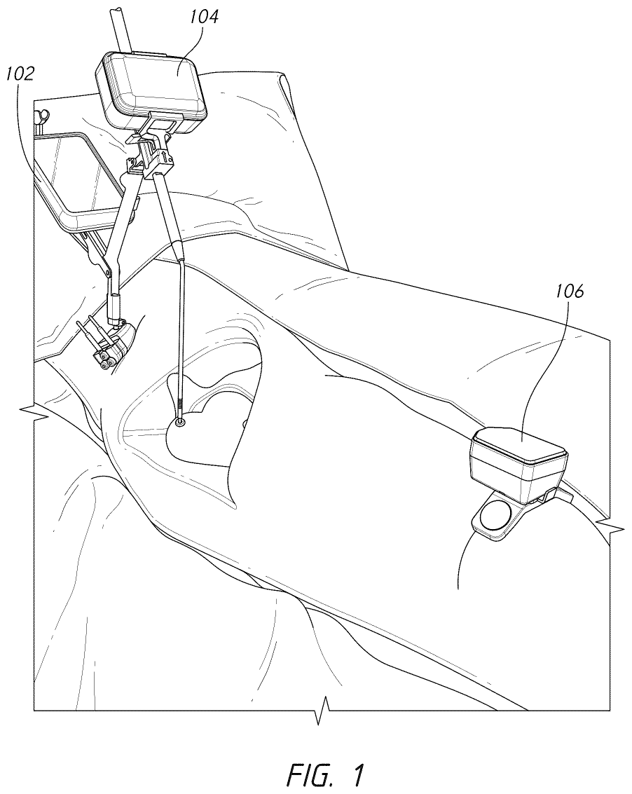

FIG. 1 shows a hip navigation system 100. The hip navigation system is configured to navigate toward optimal placement of a cup during a surgical procedure. The hip navigation system 100 is shown mounted on a pelvis for a supine approach in FIG. 1. As discussed herein, the method of use can include registering the femur prior to and after the cup is placed to confirm an aspect of the relative position and/or orientation of the femur such as leg length and joint offset.

The hip navigation system 100 can include one or more inertial sensor devices. The hip navigation system 100 includes a navigation device 102. The hip navigation system 100 includes a reference sensor device 104. The hip navigation system 100 includes a femur sensor device 106. Each of the navigation device 102, the reference sensor device 104, and/or the femur sensor device 106 can detect orientation and rotation. Each of the navigation device 102, the reference sensor device 104, and/or the femur sensor device 106 can comprise at least one sourceless sensor, such as an accelerometer, a gyroscope, or a combination of these sensors and other sensors. Each of the navigation device 102, the reference sensor device 104, and/or the femur sensor device 106 can include at least one accelerometer to detect orientation and at least one gyroscope to detect rotation.

The navigation device 102, the reference sensor device 104, and/or the femur sensor device 106 can be used to measure and record the location of points used in a hip procedure, such as the location of a point on a femur pre-and post-implantation of the cup and points that define a reference frame for cup navigation. “Navigation device”, “reference sensor device” and “femur sensor device” are broad terms and are to be given its ordinary and customary meaning to a person of ordinary skill in the art (i.e. it is not to be limited to a special or customized meaning) and includes, without limitation, any device that can be used to provide orientation information or perform orientation calculations for use in a surgical or other procedure. The navigation device 102, the reference sensor device 104, and/or the femur sensor device 106 can be configured to provide measurements for use in inertial navigation systems. The navigation device 102, the reference sensor device 104, and/or the femur sensor device 106 can provide inertial navigation to continuously calculate the position, orientation, and/or rotation of the respective device. The navigation device 102, the reference sensor device 104, and/or the femur sensor device 106 can provide inertial navigation without the need for external references.

The navigation device 102, the reference sensor device 104, and/or the femur sensor device 106 can comprise separate enclosures. The navigation device 102, the reference sensor device 104, and/or the femur sensor device 106 can be generally handheld and portable. The navigation device 102, the reference sensor device 104, and/or the femur sensor device 106 can include one or more of the following: an electronic control unit adapted to communicate with other devices, a processor, a receiver, a transmitter, a power supply, and a memory. The navigation device 102 can be configured to receive information from the reference sensor device 104 and/or the femur sensor device 106. The navigation device 102, the reference sensor device 104, and/or the femur sensor device 106 can include a plurality of sensors that together form an inertial measurement system.

The hip navigation system can include a processor. The processor is configured to register a point. The processor is configured to register the orientation and/or rotation of the navigation device 102. The processor is configured to register the orientation and/or rotation of the navigation device 102 to eliminate errors. The processor is configured to register the orientation and/or rotation of the navigation device 102 to account for movement of the pelvis. The processor is configured to register the position, orientation, and/or rotation of the reference sensor device 104. The processor is configured to register the position, orientation, and/or rotation of the reference sensor device 104 when a registration probe coupled to the reference sensor device 104 contacts a point, as described herein. The processor is configured to register the position, orientation, and/or rotation of the reference sensor device 104 when a registration probe coupled to the reference sensor device 104 contacts a divot, as described herein. The processor is configured to register the position, orientation, and/or rotation of the reference sensor device 104 when a registration probe coupled to the reference sensor device 104 contacts a femur registration marker, as described herein. The processor is configured to register the position, orientation, and/or rotation of the reference sensor device 104 when registering a reference frame. The processor is configured to register the orientation and/or rotation of the femur sensor device 106. The processor is configured to register the orientation and/or rotation of the femur sensor device 106 when the femur sensor is coupled to the greater trochanter. The processor is configured to register the orientation and/or rotation of the femur sensor device 106 when the femur sensor is coupled to the distal thigh. The processor is configured to register the orientation and/or rotation of the femur sensor device 106 when the femur sensor is coupled to an impactor adapter, as described herein. The processor is configured to register the orientation and/or rotation of the femur sensor device 106 during cup navigation. The processor is configured to register points of a reference frame. The processor is configured to store a reference frame. The processor is configured to calculate angles relative to the reference frame. The processor is configured to register a point before cup placement. The processor is configured to register a point after cup placement. The processor is configured to mathematically rotate a femur about a center of rotation. The processor is configured to determine changes in leg length. The processor is configured to determine changes in joint offset. In some embodiments, the navigation device 102 comprises the processor configured to perform one or more functions described herein. In some embodiments, the reference sensor device 104 comprises the processor configured to perform one or more functions described herein. In some embodiments, the femur sensor device 106 comprises the processor configured to perform one or more functions described herein.

The navigation device 102, the reference sensor device 104, and/or the femur sensor device 106 can include a first sensor or sensors for determining acceleration. The navigation device 102, the reference sensor device 104, and/or the femur sensor device 106 can include a second sensor or sensors for determining gyroscopic positioning. The navigation device 102, the reference sensor device 104, and/or the femur sensor device 106 can include one or more accelerometers. The navigation device 102, the reference sensor device 104, and/or the femur sensor device 106 can include one or more gyroscopic sensors. The navigation device 102, the reference sensor device 104, and/or the femur sensor device 106 can include at least one sourceless sensor, such as an accelerometer, a gyroscope, or a combination of these sensors and other sensors. The navigation device 102, the reference sensor device 104, and/or the femur sensor device 106 can includes a three-axis accelerometer to detect orientation relative to gravity. The navigation device 102, the reference sensor device 104, and/or the femur sensor device 106 can include a plurality of gyroscopes to detect rotation.

The navigation device 102, the reference sensor device 104, and/or the femur sensor device 106 can include a transmitter for sending data from the respective device to another device. The navigation device 102, the reference sensor device 104, and/or the femur sensor device 106 can include a receiver for receiving data from another device. In some embodiments, data from the navigation device 102, the reference sensor device 104, and/or the femur sensor device 106 can correspond to the respective rotation and/or respective orientation and/or respective position the navigation device 102, the reference sensor device 104, and/or the femur sensor device 106. In some embodiments, data from the reference sensor device 104 and/or the femur sensor device 106 can be used by the navigation device 102 to determine an aggregate, or overall, position, orientation, and/or rotation of components of the hip navigation system 100. In some embodiments, data from the reference sensor device 104 and/or the femur sensor device 106 can be used by the navigation device 102 to determine an aggregate, or overall, position, orientation, and/or rotation of the corresponding anatomy of the patient.

The navigation device 102, the reference sensor device 104, and/or the femur sensor device 106 can provide a measurement of the center of the rotation. The navigation device 102, the reference sensor device 104, and/or the femur sensor device 106 can provide a measurement of or can determine the direction of the femur. The navigation device 102, the reference sensor device 104, and/or the femur sensor device 106 can provide a measurement of or can determine a location of or relative position of points that define a reference frame. The navigation device 102, the reference sensor device 104, and/or the femur sensor device 106 can provide a measurement of or locate a gravity vector within a reference frame.

The data from the navigation device 102, the reference sensor device 104, and/or the femur sensor device 106 can be input to one or more microprocessors. The navigation device 102, the reference sensor device 104, and/or the femur sensor device 106 can comprise one or more microprocessors. In some embodiments, the navigation device 102, the reference sensor device 104, and/or the femur sensor device 106 each includes a microprocessor. In some embodiments, the navigation device 102 includes the microprocessor for processing data for the hip navigation system 100. In another embodiment, the reference sensor device 104 includes the microprocessor for processing data for the hip navigation system 100. In another embodiment, the femur sensor device 106 includes the microprocessor for processing data for the hip navigation system 100. In some methods, an algorithm implemented on the microprocessor can process data in real time.

The navigation device 102 can include a graphical user interface 108. The graphical user interface 108 of the navigation device 102 can illustrate one or more angles of the cup relative to a reference frame acquired by the hip navigation system 100. The graphical user interface 108 of the navigation device 102 can illustrate one or more of abduction and anteversion angles. The graphical user interface 108 can include an interactive window displaying on-screen graphics. The graphical user interface 108 can provide the user with a plurality of screens illustrating steps to be performed in a surgical procedure. The graphical user interface 108 can guide the user through the performance of the steps. Each screen can comprise one or more on-screen graphics. The on-screen graphics can comprise one or more visual cues or indicators to prompt the user as to what step or steps to take next during one of the procedural methods described herein. The visual cues of the graphical user interface 108 can include instructive images, diagrams, pictorial representations, icons, animations, visual cues, charts, numerical readings, measurements, textual instructions, warnings (visual and/or audible), or other data. The graphical user interface 108 can be configured to alter attributes (e.g., color) of the on-screen graphics according to one or more data protocols. The graphical user interface 108 can provide visual feedback to the user during performance of one or more steps of a surgical procedure. The graphical user interface 108 can be configured to generate GUI images to be displayed to the user. The graphical user interface 108 of the navigation device 102 can depict images of the surgical procedure. The graphical user interface 108 of the navigation device 102 can depict an image of the navigation device 102.

The graphical user interface 108 of the navigation device 102 can include a touch screen display. The user can interact with the navigation device 102 via buttons, switches, touch screen buttons, scroll wheel, track ball, keyboard, remote controls, and/or a microphone in conjunction with speech recognition software. The graphical user interface 108 can allow a user to confirm that a step has been completed, for example, by pressing a button on the touchscreen display. The graphical user interface 108 can allow the user to enter data such as a numerical value, a measurement from a pre-operative image, a distance, and/or a target angle. The graphical user interface 108 can allow the user to enter data to register the orientation of the navigation device 102, the reference sensor device 104, and/or the femur sensor device 106. In some embodiments, the navigation device 102 comprises the graphical user interface 108. In some embodiments, the reference sensor device 104 comprises the graphical user interface 108. In some embodiments, the femur sensor device 106 comprises the graphical user interface 108. In some embodiments, another device, such as a tablet, smartphone or other computing device configured to be positioned in the surgical field, comprises the graphical user interface 108.

The navigation device 102, the reference sensor device 104, and/or the femur sensor device 106 can be paired such that the devices are in wireless communication with each other. This permits one of the navigation device 102, the reference sensor device 104, and/or the femur sensor device 106 to control one or more other devices, store data from one or more other device, and/or display information based on signals from one or more other devices. In some embodiments, the graphical user interface 108 of the navigation device 102 confirms to the user certain angles based on the data sensed by the reference sensor device 104 and/or the femur sensor device 106. The graphical user interface 108 of the navigation device 102 can illustrate angles indicative of the orientation of the cup, e.g., degrees of abduction and anteversion with respect to the selected reference frame. The angle displayed can directly reflect one or more reference frames described herein. The angle displayed can directly reflect the reference frame from one or more of the acquisition of landmarks, gravity, and/or measurements from pre-operative imaging. In some embodiments, the graphical user interface 108 of the navigation device 102 confirms to the user the orientation and/or rotation of the femur based on the data sensed by the reference sensor device 104 and/or the femur sensor device 106. The pairing of the navigation device 102, the reference sensor device 104, and/or the femur sensor device 106 can be calibrated as described herein.

The hip navigation system 100 can be used in a surgical procedure. While some methods are shown as views on a screen of the graphical user interface 108 of the navigation device 102, the corresponding steps can be performed by a user during surgical methods. While some methods are shown with respect to the patient, the corresponding steps can be displayed on a screen of the graphical user interface 108 of the navigation device 102. The hip navigation system 100 can be used in a supine technique. The hip navigation system 100 can be used for total hip arthroplasty. The hip navigation system 100 can be used to assist the user in navigating acetabular cup abduction and anteversion angles for proper cup placement. The hip navigation system 100 can be used to assist the user in measuring changes in leg length and offset. The hip navigation system 100 can be used with software installed on the navigation device 102, the reference sensor device 104, and/or the femur sensor device 106. The software version installed on the devices can be available to view via a menu on a screen of the graphical user interface 108. The navigation device 102, the reference sensor device 104, and the femur sensor device 106 can be used in conjunction with mechanical instrumentation from the instrument tray described herein. The hip navigation system 100 provides users with a user-friendly, cost-effective, surgical navigation system for precise alignment.

The hip navigation system 100 can be a system intended to assist the user in determining reference alignment axes in relation to anatomical and instrumentation structures during stereotactic orthopedic surgical procedures. The hip navigation system 100 can facilitate the accurate positioning of implants relative to these alignment axes. The hip navigation system 100 aids the user in controlling leg length and joint offset discrepancies. Example orthopedic surgical procedures include but are not limited to total or partial hip arthroplasty. Other orthopedic surgical procedures include but are not limited to total or partial knee arthroplasty and total or partial shoulder arthroplasty. Example orthopedic surgical procedures include but are not limited to the supine approach.

In some embodiments, the navigation device 102 is not intended to be re-sterilized. The navigation device 102 can be provided sterile for single use only. The navigation device 102 can contain electronic components that could be damaged by sterilization. In some methods, the navigation device 102 is discarded after use. In some methods, the navigation device 102, the reference sensor device 104, and/or the femur sensor device 106 comprise inertial sensors and should not be used if dropped. In some embodiments, the reference sensor device 104 and/or the femur sensor device 106 can be re-sterilized for additional surgical procedures. In some methods, the reference sensor device 104 and/or the femur sensor device 106 can include a battery that is to be removed and discarded prior to sterilization. The battery must be installed within the reference sensor device 104 and/or the femur sensor device 106 per the markings on the respective device. The battery must be inserted in the correct orientation and fully seated to power on the respective device.

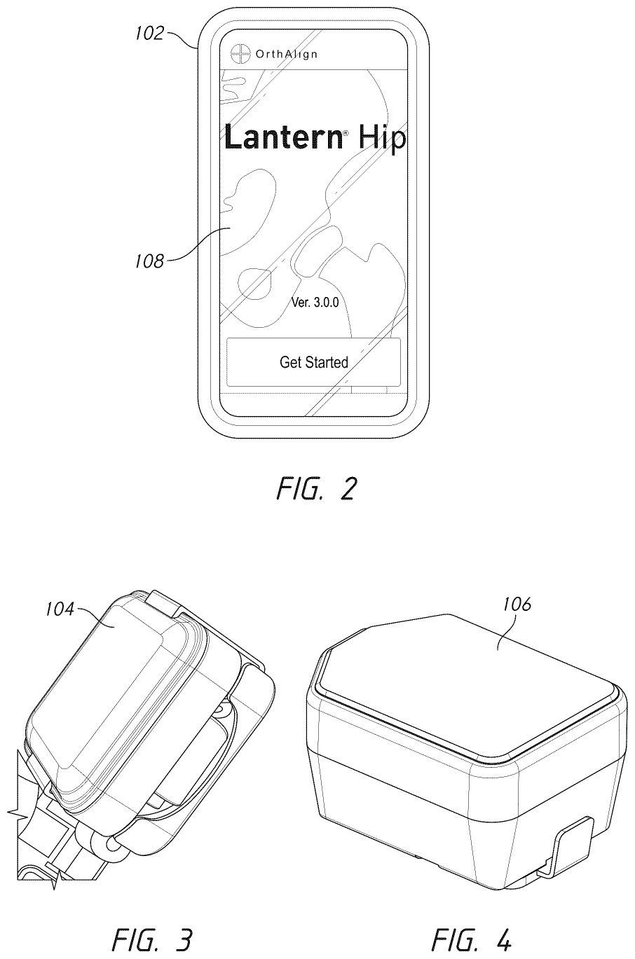

The hip navigation system 100 can include three electronic components to be used in conjunction with instrumentation from the tray as described herein. The hip navigation system 100 includes the navigation device 102. FIG. 2 is a front view of the navigation device 102 of the hip navigation system 100. The hip navigation system 100 includes the reference sensor device 104. FIG. 3 is a perspective view of the reference sensor device 104 of the hip navigation system 100. The hip navigation system 100 includes the femur sensor device 106. FIG. 4 is a side view of the femur sensor device 106 of the hip navigation system 100.

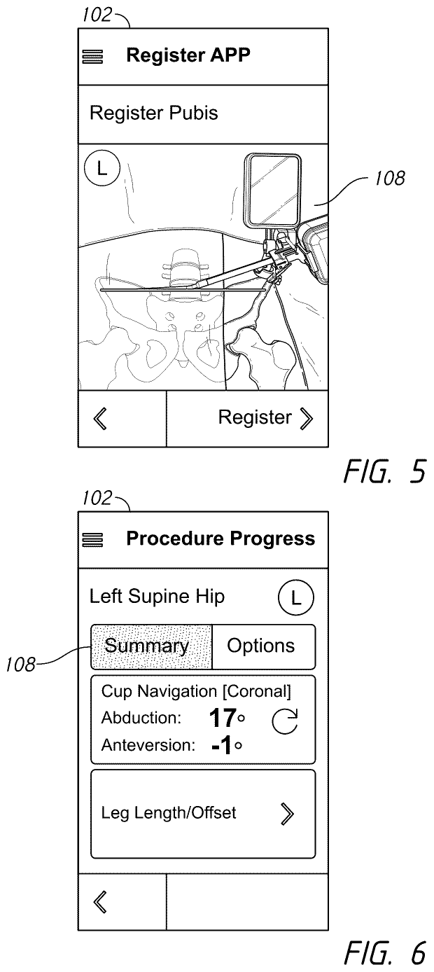

The navigation device 102 includes the graphical user interface 108 as shown in FIG. 2. The navigation device 102 can be a touchscreen device. To move through the workflow, the user can tap the prompts as they appear on the graphical user interface 108. While the following figures illustrate methods according to some embodiments, the methods can include one or more of the following steps and the methods can omit one or more of the following steps. While the following figures illustrate methods in a particular order according to some embodiments, the methods can include steps in any order. While the following figures illustrate methods according to some embodiments, the methods can include alternatives such as alternative placements of the femur sensor device 106 during leg length and joint offset. FIGS. 5-8 are examples of screens of the graphical user interface 108 of the navigation device 102 viewable during the surgical procedure.

FIG. 5 is a view of a screen of the graphical user interface 108 of a component of the hip navigation system 100. Disclosed herein are various screens of the graphical user interface 108. These screens are displayed on the navigation device 102 of FIG. 2 in one embodiment. These screens could be displayed on any of the sensor devices disclosed herein, such as the navigation device 102, the reference sensor device 104, and/or the femur sensor device 106. These screens could be displayed on another device, such as a tablet, smartphone or other mobile computing device configured to be positioned in the sterile field. The screen can be a register pubis screen. The graphical user interface 108 can include a menu, which can be tapped to expand the menu. The graphical user interface 108 can include a left/right icon, which indicates the operative side. The graphical user interface 108 can include a back arrow, which the user can tap to move to the previous step. The graphical user interface 108 can include a title bar to view the current procedure section. The graphical user interface 108 can include a step display to view the current procedure step. The graphical user interface 108 can include an animation to view the current workflow step. The graphical user interface 108 can include an advance arrow, which the user can tap to register or proceed to move to the next step.

FIG. 6 is a view of a screen of the graphical user interface 108 of the navigation device 102. The screen can be a procedure progress screen. The graphical user interface 108 can include a cup measurements button to view the recorded cup values. The graphical user interface 108 can include an options button, which the user can tap to view further options. The graphical user interface 108 can include a repeat icon, which the user can tap to repeat cup measurements. The graphical user interface 108 can include an advance arrow, which the user can tap to proceed to leg length and offset.

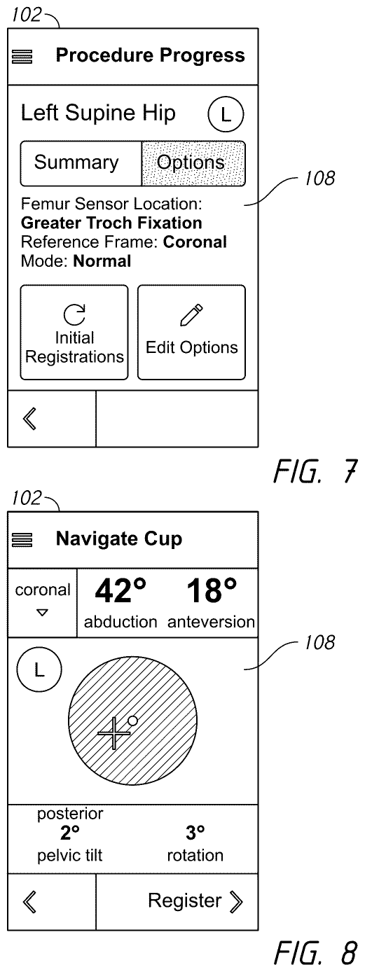

FIG. 7 is a view of a screen of the graphical user interface 108 of the navigation device 102. The screen can be a procedure progress screen, options tab. The graphical user interface 108 can include a summary button, which the user can tap to return to the procedure progress summary tab. The graphical user interface 108 can include a repeat button, which the user can tap to repeat initial instructions. The graphical user interface 108 can include an overview to view current surgical preferences. The graphical user interface 108 can include an edit options button, which the user can tap to edit surgical preference options.

FIG. 8 is a view of a screen of the graphical user interface 108 of the navigation device 102. The screen can be a navigate cup screen. The graphical user interface 108 can include a dropdown, which the user can tap to view additional reference frames. The graphical user interface 108 can allow the user to view real-time pelvic tilt and rotation. The graphical user interface 108 can allow the user to view real time cup abduction and anteversion angles. The graphical user interface 108 can include a cup target to view cup position in relation to the user's previously set target abduction and anteversion angles, represented by the center of the target. The graphical user interface 108 can include an advance arrow, which the user can tap to register to save measurements and move to the next step.

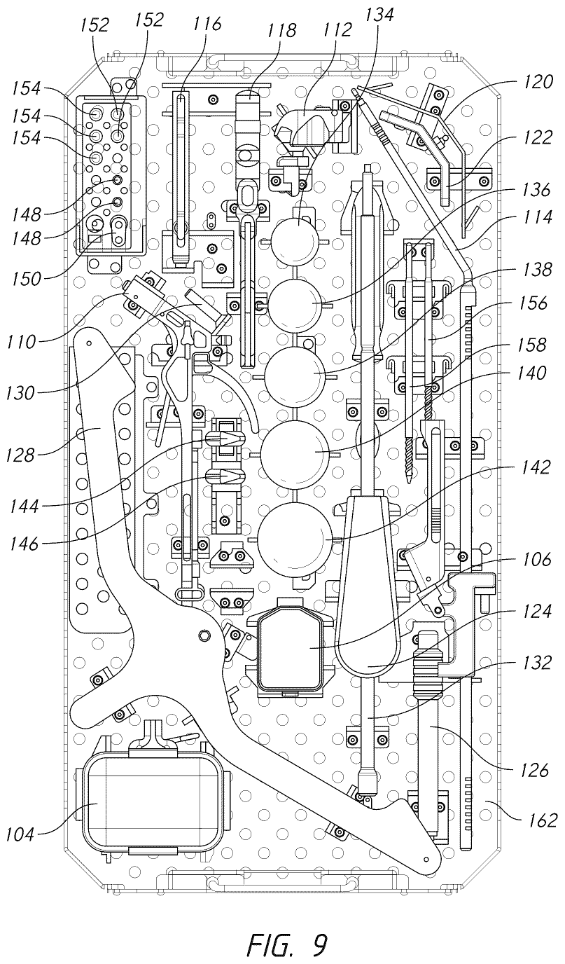

FIG. 9 is a view of the components of an embodiment of a kit including at least some of one embodiment of the hip navigation system 100 disposed within a tray. The tray can include reusable components. The tray can include component that are configured to be sterilized. The navigation device 102 can be separately provided from the tray. The navigation device 102 can be single use. The navigation device 102 can be disposable. Additional components of the hip navigation system can be disposable, such as batteries. The hip navigation system 100 can include the reference sensor device 104. The hip navigation system 100 can include the femur sensor device 106. The hip navigation system 100 can include a pelvic bracket 110. The hip navigation system 100 can include a pelvic base 112. The hip navigation system 100 can include a registration probe 114. The hip navigation system 100 can include a fixation tower 116. The hip navigation system 100 can include a fixation inserter handle 118. The hip navigation system 100 can include a thigh plate 120. The hip navigation system 100 can include a thigh mount 122. The thigh mount 122 can be magnetic. The hip navigation system 100 can include a hex driver 124. The hex driver 124 can be a 3.5 mm teardrop hex driver. The hip navigation system 100 can include a holding sleeve 126. The hip navigation system 100 can include a probe calibration jig 128. The probe calibration jig 128 can be a tripod. The hip navigation system 100 can include a drill guide 130. The drill guide 130 can be a 4 mm diameter drill guide. The hip navigation system 100 can include a registration wand 132. The hip navigation system 100 can include a first liner registration trial 134. The first liner registration trial 134 can have a diameter of 28 mm. The hip navigation system 100 can include a second liner registration trial 136. The second liner registration trial 136 can have a diameter of 32 mm. The hip navigation system 100 can include a third liner registration trial 138. The third liner registration trial 138 can have a diameter of 36 mm. The hip navigation system 100 can include a fourth liner registration trial 140. The fourth liner registration trial 140 can have a diameter of 40 mm. The hip navigation system 100 can include a fifth liner registration trial 142. The fifth liner registration trial 142 can have a diameter of 44 mm. The hip navigation system 100 can include an impactor adapter clip 144. The hip navigation system 100 can include an additional impactor adapter clip 146. The hip navigation system 100 can include one or more fixation screws 148. The hip navigation system 100 can include a fixation plate 150. The fixation plate 150 can be 5 mm. The hip navigation system 100 can include one or more screws 152. The screw 152 can be a 4.5 mm diameter×20 mm, 3.5 mm hex head. The hip navigation system 100 can include one or more femur registration markers 154. The hip navigation system 100 can include a fixation pin 156 and a fixation pin 158. The threaded fixation pins 156, 158 can be Ø4.0×110 mm or Ø4.0×140 mm with Ø3.2mm driver end. The hip navigation system 100 can include a tray base 162. The hip navigation system 100 can include a tray lid configured to couple to the tray base 162.

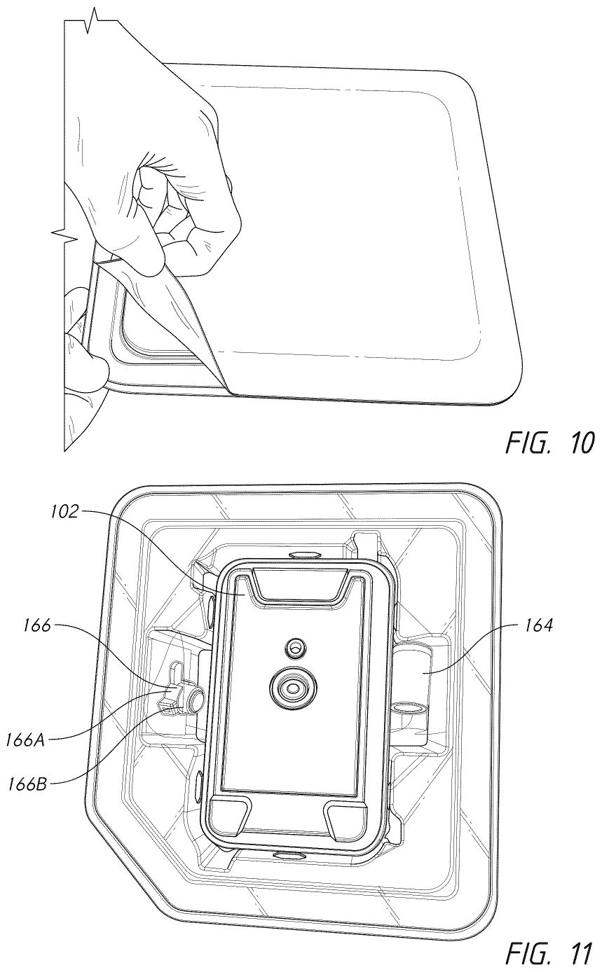

In some embodiments, the methods of use of the hip navigation system 100 can include back table set up. In some embodiments, the methods of use of the hip navigation system 100 can include electronic pairing and set up. FIG. 10 is a view of outer packaging of the navigation device 102. The method can include opening the outer packaging of the navigation device 102. The method can include passing the sterile inner packaging onto the sterile field. FIG. 11 is a view of inner packaging of the navigation device 102. The method can include opening the inner packaging of the navigation device 102. The method can include opening the inner packaging to expose the navigation device 102, a reference sensor battery 164, and a femur sensor battery assembly 166. The femur sensor battery assembly 166 includes a cap device 166A and a battery 166B. The cap device 166A comprises a recess into which one end of the battery 166B can be inserted. The cap device 166A includes one or more, e.g., two, flanges extending away from a closed end of the recess. The flanges can extend from diametrically opposed portions of the periphery of the closed end of the recess. The cap device 166A can include a removal tab extending from a side surface of the cap device 166A. The removal tab can receive a load (e.g., from a thumb of the user) to urge the cap device 166A and thus the battery assembly 166 out of the femur sensor device 106. The cap device 166A can be T-shaped, with a shell forming the recess being the vertical portion of the T and the flanges being the horizontal portions of the T. The removal tab can extend along the vertical direction of the T. The battery assembly 166 can be inserted into a T-shaped cavity in the housing of the femur sensor device 106. When so inserted the battery 166B makes contact with an electrical circuit of the femur sensor device 106. When so inserted the flanges and the removal tab are positioned in correspondingly shaped portions of the T-shaped cavity of the femur sensor device 106. One or more of the flanges and the removal tab can form flush surfaces of the femur sensor device 106 when the battery assembly 166 is inserted. The removal tab may project from an end surface to facilitate a thumb or other finger of the user applying a load to the femur sensor battery assembly 166 to remove the femur sensor battery assembly 166 from the femur sensor device 106.

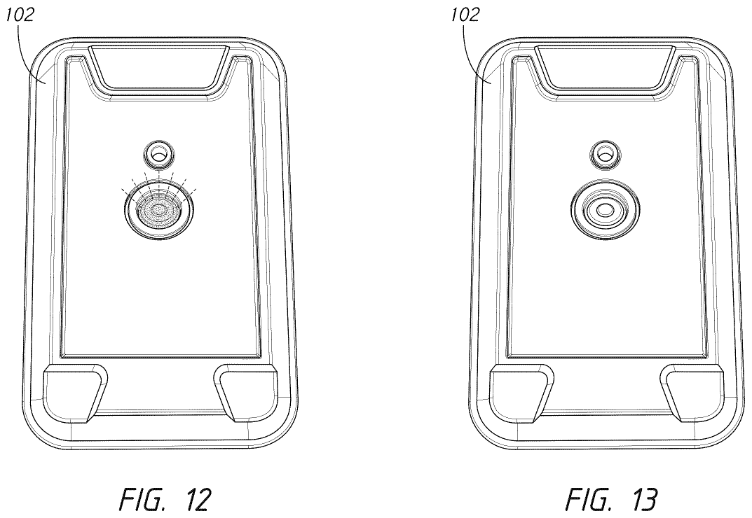

The method can include powering on the navigation device 102. The user can press and hold the power button on the back of the navigation device 102 for approximately 3 seconds. The power button can start blinking when the navigation device 102 initiates bootup. The navigation device 102 can take several seconds to display the bootup animation on the graphical user interface 108. The navigation device 102 can include a pre-installed battery with a battery life of at least 2.5 hours. The graphical user interface 108 can be configured to dim after approximately 3 minutes of inactivity to preserve power. FIG. 12 is a view of the navigation device 102 with the power button on. FIG. 13 is a view of the navigation device 102 with the power button off.

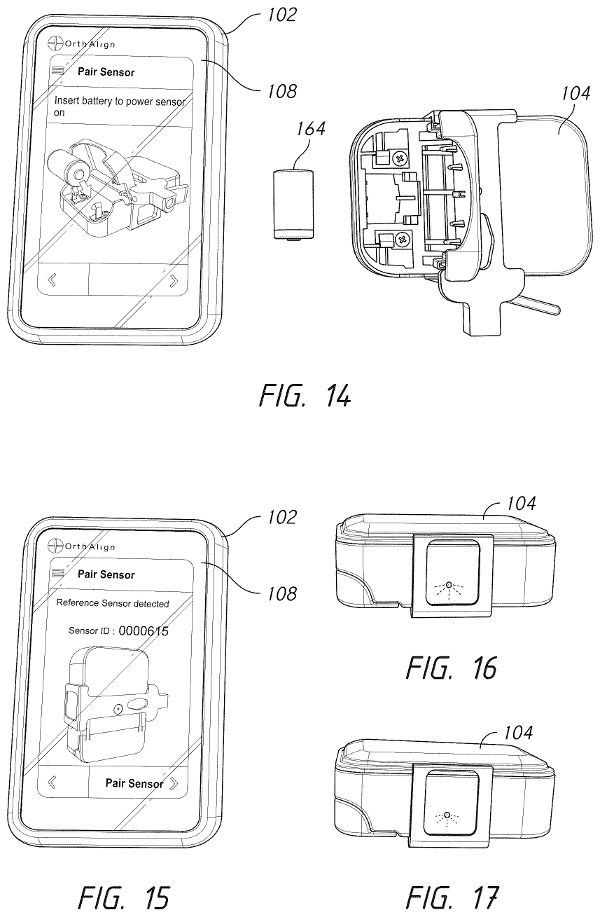

The method can include powering on the reference sensor device 104. The user can insert the reference sensor battery 164 into the reference sensor device 104, taking care to match the part markings visible under the battery cover. Once the reference sensor battery 164 is installed, the user can close the cover of the reference sensor device 104 and observe the LED of the reference sensor device 104. The LED of the reference sensor device 104 will blink amber to indicate that the reference sensor battery 164 has been inserted correctly. FIG. 14 is a view of the navigation device 102, the reference sensor battery 164, and the reference sensor device 104.

The method can include pairing the reference sensor device 104 with the navigation device 102. Once prompted, the user can select the reference sensor identification number from a drop-down menu on the graphical user interface 108 of the navigation device 102 that matches the identification number on the reference sensor device 104. The user can tap the pair sensor button on the graphical user interface 108 of the navigation device 102. The user can observe the green blinking LED when the reference sensor device 104 is successfully paired with the navigation device 102. FIG. 15 is a view of a screen of the graphical user interface 108 of the navigation device 102 displaying the pair reference sensor screen. FIG. 16 is a view of the reference sensor device 104 which is powered on, but not paired. The LED light is amber indicating that the reference sensor battery 164 has been inserted correctly. FIG. 17 is a view of the reference sensor device 104, which is powered on and paired. The LED light is green indicating that the reference sensor device 104 is paired with the navigation device 102.

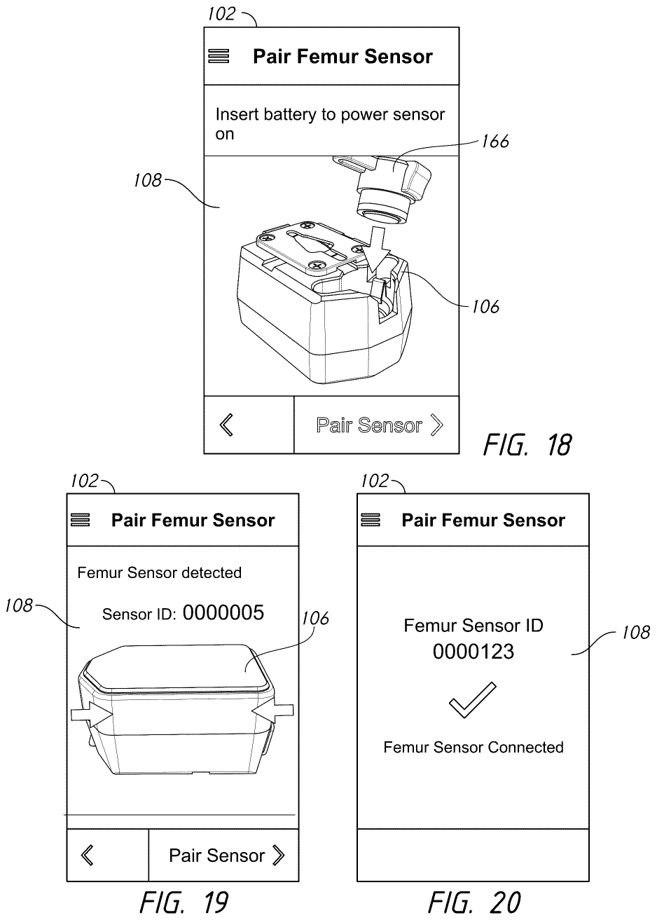

The method can include powering on the femur sensor device 106. The method can include inserting the femur sensor battery assembly 166 into the femur sensor device 106. The graphical user interface 108 of the navigation device 102 illustrates inserting the femur sensor battery assembly 166 into the femur sensor device 106. The user can ensure that the femur sensor battery assembly 166 is fully inserted into the battery cavity. Inserting the femur sensor battery assembly 166 can include rotationally aligning the cap device 166A, e.g., the flanges and/or the removal tab, with corresponding portions of a cavity of the housing of the femur sensor 106. The cap device 166A is rotationally asymmetric. The flanges of the femur sensor battery assembly 166 and the cavity of the housing of the femur sensor 106 can only mate in one rotational orientation in one embodiment. The removal tab of the femur sensor battery assembly 166 and the cavity of the housing of the femur sensor 106 can only mate in one rotational orientation in one embodiment. The rotational asymmetry facilitates quick assembly of the battery assembly 166 with the housing of the femur sensor device 106, keeping assembly and set up time to a minimum, which helps streamline and shorten the process of providing treatment to the patient. Upon successful insertion, the user can receive tactile feedback such as a feeling a click. The LED of the femur sensor device can blink amber to indicate that the femur sensor battery assembly 166 is inserted correctly. FIG. 18 is a view of a screen of the graphical user interface 108 of the navigation device 102. The graphical user interface 108 can provide an animation to direct the user to correctly orient the femur sensor battery 166.

The method can include pairing the femur sensor device 106 with the navigation device 102. In some methods, the user pairs the reference sensor device 104 before the femur sensor device 102. After pairing the reference sensor device 104, the navigation device 102 will begin searching for the femur sensor device 106. Once prompted, the user can select the femur sensor identification number from a drop-down menu on the graphical user interface 108 of the navigation device 102 that matches the identification number on the femur sensor device 106. The user can tap the pair sensor button on the graphical user interface 108 of the navigation device 102. The user can observe the green blinking LED on the femur sensor device 106 when the femur sensor device 106 is successfully paired with the navigation device 102.

FIG. 19 is a view of a screen of the graphical user interface 108 of the navigation device 102 displaying the pair femur sensor screen. The graphical user interface 108 can provide an animation to direct the user to find the femur sensor identification number located on the femur sensor device 106. FIG. 20 is a view of a screen of the graphical user interface 108 of the navigation device 102 indicating that the femur sensor device 106 is successfully paired with the navigation device 102.

The user can interact with the graphical user interface 108 of the navigation device 102 for procedural selection. The user can complete procedure selections in the procedure options screens, according to their preferences. FIG. 21 is a view of a screen of the graphical user interface 108 of the navigation device 102. The user can select operative side, either left or right. The user can toggle the button to turn advanced mode on or off. With the advanced mode on, the graphical user interface 108 of the navigation device 102 can display more streamlined instructions with fewer screens. With the advanced mode off, the graphical user interface 108 of the navigation device 102 can display additional animations and guidance.

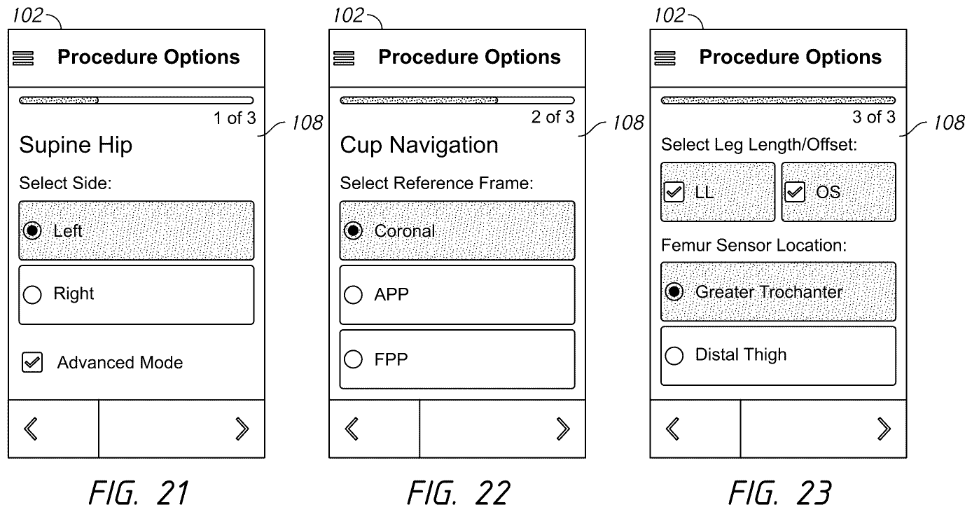

FIG. 22 is a view of a screen of the graphical user interface 108 of the navigation device 102. The user can select the reference frame for cup navigation. The reference frame can be the anterior pelvic plane (APP). The reference frame can be a functional pelvic plane (FPP). The functional pelvic plane can be determined by acquiring additional inputs based on patient anatomy. The functional pelvic plane can be determined from a measurement of pelvic tilt during standing lateral X-ray which must be determined pre-operatively. The measurement of pelvic tilt can be entered by the user into the graphical user interface 108 of the navigation device 102. The functional pelvic plane can allow patient specific cup placement. In some methods, if the functional pelvic plane is not set up during this step, then the functional pelvic plane will not be visible during the procedure. The reference frame can be a coronal plane. While the selected reference frame can be the reference frame for the primary display, the user may view all reference frames by tapping the dropdown arrow on the graphical user interface 108 of the navigation device 102 during cup navigation.

FIG. 23 is a view of a screen of a graphical user interface 108 of the navigation device 102. The user can select to measure leg length. The user can select to measure joint offset. If leg length and/or joint offset are selected, in some methods, then the user is prompted to also select a location. The user can select the location for fixation of the femur sensor 106 during leg length and/or joint offset measurements. The locations can be the greater trochanter or the distal thigh. In some methods, the surgical procedure is limited to the distal thigh location and the user does not need to select a location.

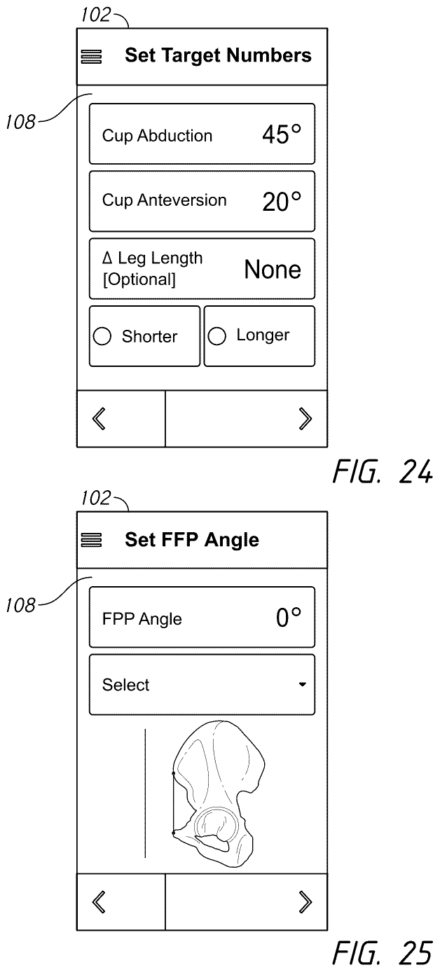

The method can include completing procedure selection in one or more screens, according to preferences of the user. FIG. 24 is a view of a screen of the graphical user interface 108 of the navigation device 102. The user can set target numbers. The user can set a target cup abduction angle. The user can set a target cup anteversion angle. The user can set a target change in leg length. The user can select whether a target change in leg length is shorter or longer. In some methods, the inputted numbers do not restrict the user to a specific plan, but instead provide feedback on subsequent live navigation screens.

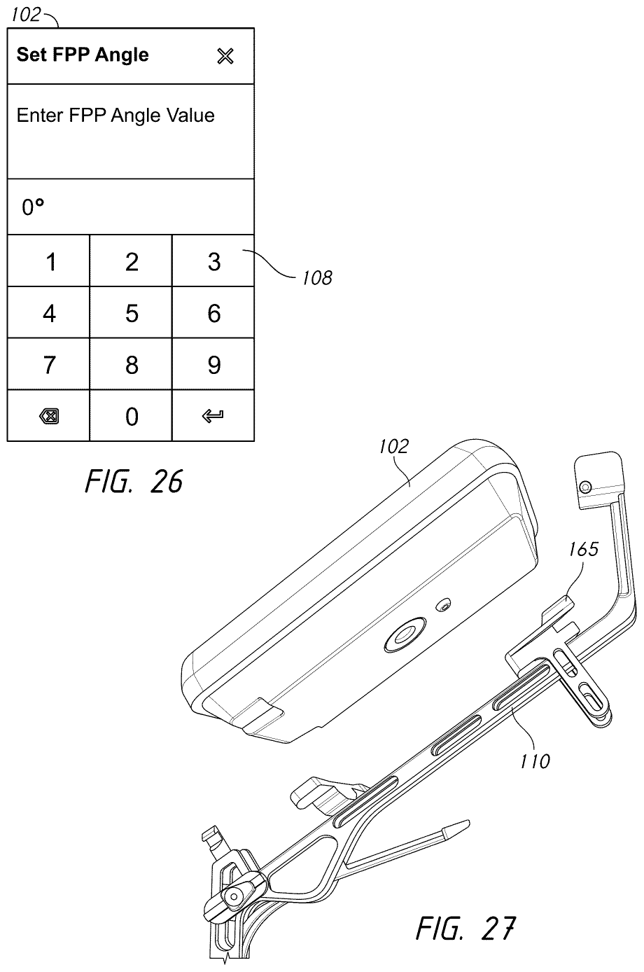

FIG. 25 is a view of a screen of the graphical user interface 108 of the navigation device 102. The user can set a functional pelvic plane angle for the patient. The functional pelvic plane can be determined from a measurement of pelvic tilt from a pre-operative image. The user can enter a numerical value for the angle. The user can select anterior or posterior. If functional pelvic plane was selected as a reference frame, then the user must also set the patient's functional pelvic plane angle. FIG. 26 is a view of a screen of the graphical user interface 108 of the navigation device 102. The user can enter the angle on a keypad screen of the graphical user interface 108 of the navigation device 102. The functional pelvic plane angle can be measured via standard practices using the patient's standing lateral X-ray. The functional pelvic plane angle is the angle between a vertical line and the patient's anterior pelvic plane drawn between the inter-ASIS line and pubic tubercle. The acceptable range of the functional pelvic plane angle can be between 0° and 30°.

The method can include calibration. FIG. 27 is a view of the navigation device 102. The method can include attaching the devices to the pelvic bracket 110. The graphical user interface 108 of the navigation device 102 can display animations illustrating how to attach the devices. The navigation device 102 can be coupled to the pelvic bracket 110. The pelvic bracket 110 can include a magnetic section 165. The navigation device 102 can have a corresponding magnetic section configured to securely couple to the magnetic section 165 of the pelvic bracket 110.

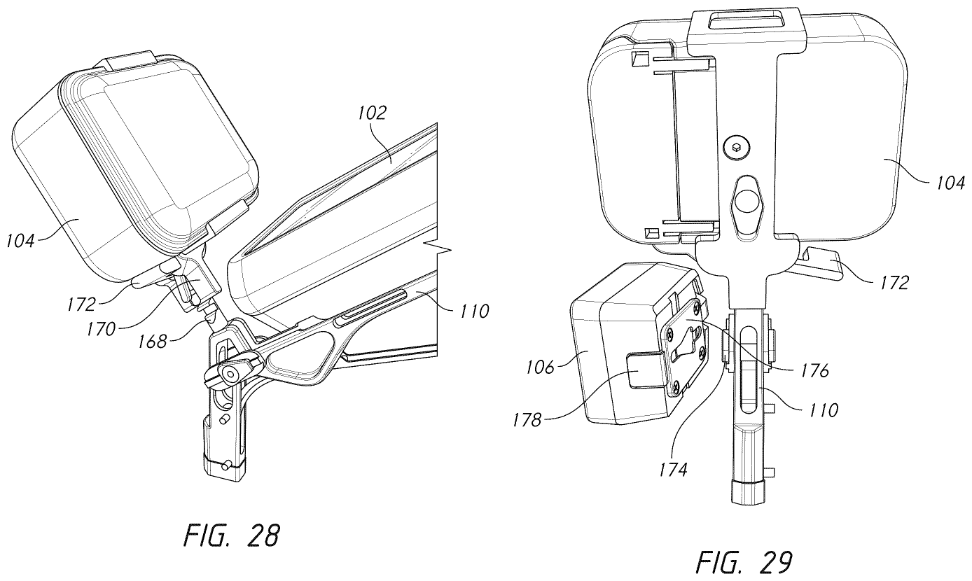

FIG. 28 is a view of the navigation device 102 and the reference sensor device 104. The reference sensor device 104 can be coupled to the pelvic bracket 110. The pelvic bracket 110 can include a coupler 168. The reference sensor device 104 can have a corresponding receiver 170 configured to securely couple to the coupler 168 of the pelvic bracket 110. The user can depress a lever 172 on the reference sensor device 104 to engage the coupler 168 of the pelvic bracket 110.

FIG. 29 is a view of the reference sensor device 104 and the femur sensor device 106. The femur sensor device 106 can be coupled to the pelvic bracket 110. The pelvic bracket 110 can include a coupler 174. The femur sensor device 106 can have a corresponding receiver 176 configured to securely couple to the coupler 174 of the pelvic bracket 110. The user can depress a lever 178 on the femur sensor device 106 to engage the coupler 174 of the pelvic bracket 110. The femur sensor device 106 can be coupled to the coupler 174 on the left side of the pelvic bracket 110. The femur sensor device 106 can be positioned on the left side of the pelvic bracket 110 for easier access to the lever 172 of the reference sensor 104. The lever 172 and the lever 178 can be on opposite sides of the pelvic bracket 110. The levers 172, 178 are easily accessible by the user during the procedure. The lever 178 of the femur sensor device 106 can be depressed to facilitate the femur sensor device 106 engaging the coupler 174 of the pelvic bracket 110. The navigation unit 102 is positioned behind the reference sensor device 104. The user can ensure the foot of the lever 178 of the femur sensor device 106 is pointed away from the navigation unit 102.

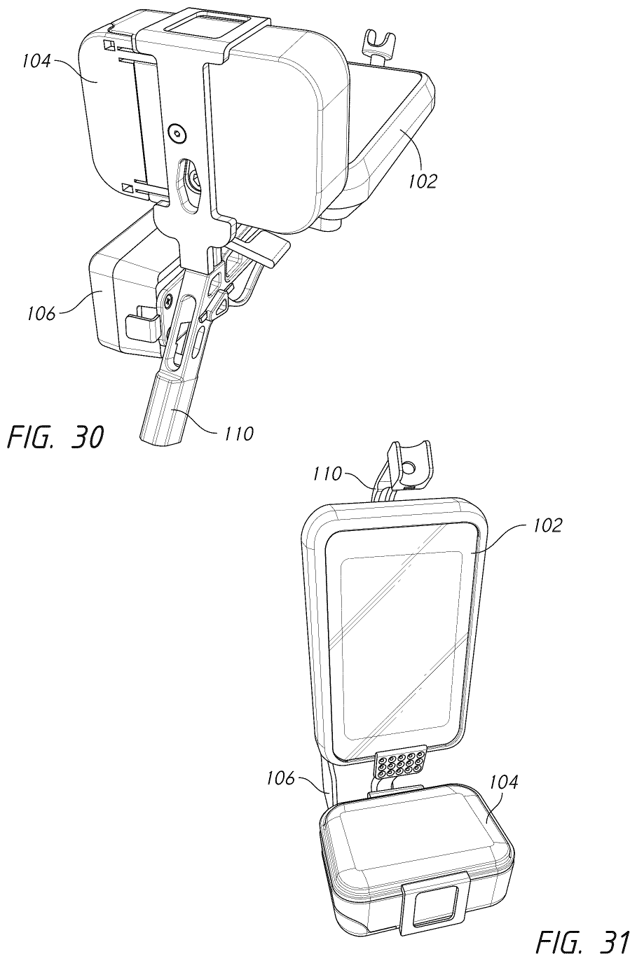

The method can include calibration. The user can complete device calibration using animations displayed on the graphical user interface 108 of the navigation device 102. The navigation unit 102, the reference sensor device 104, and the femur sensor device 106 can be coupled to the pelvic bracket 110 during calibration. The navigation unit 102, the reference sensor device 104, and the femur sensor device 106 can be in a fixed relationship to each other during calibration. The user can use a flat table surface for calibration. The flat table surface, instead of a Mayo stand, can provide better stability and calibration. FIG. 30 is a view of the navigation device 102, the reference sensor device 104, and the femur sensor device 106. The navigation device 102, the reference sensor device 104, and the femur sensor device 106 can be couped to the pelvic bracket 110 for calibration. The method can include placing the navigation device 102 flat on the table. The method can include waiting for an audible beep. The beep can indicate that the user can proceed to the next calibration step.

FIG. 31 is a view of the navigation device 102, the reference sensor device 104, and the femur sensor device 106. The navigation device 102, the reference sensor device 104, and the femur sensor device 106 can be couped to the pelvic bracket 110 for calibration. The method can include placing the navigation device 102 vertical. The method can include waiting for an audible beep. The beep can indicate that the user can proceed to the next calibration step.

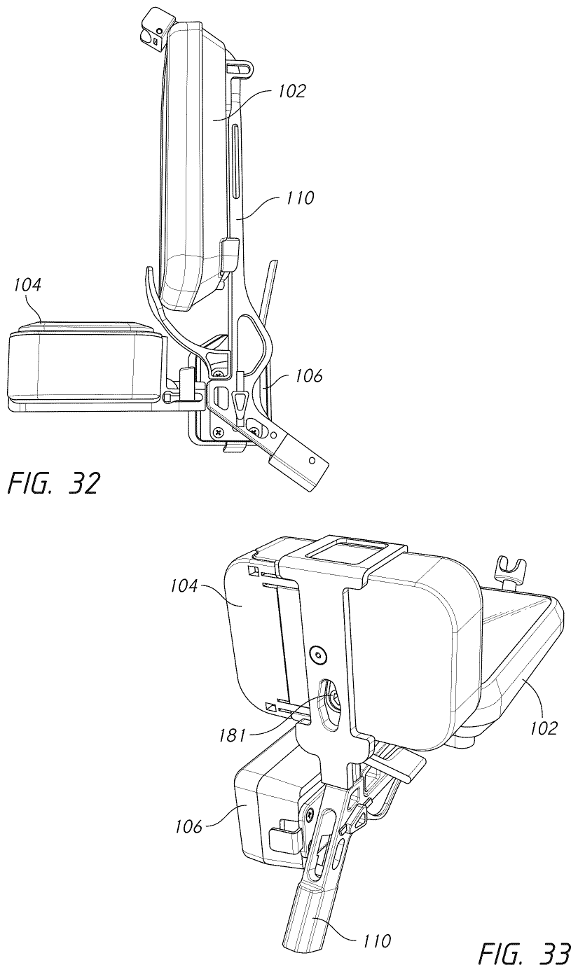

FIG. 32 is a view of the navigation device 102, the reference sensor device 104, and the femur sensor device 106. The navigation device 102, the reference sensor device 104, and the femur sensor device 106 can be couped to the pelvic bracket 110 for calibration. The method can include placing the navigation device 102 on its left side. The method can include waiting for an audible beep. The beep can indicate that the user can proceed to the next calibration step.

FIG. 33 is a view of the navigation device 102, the reference sensor device 104, and the femur sensor device 106. The navigation device 102, the reference sensor device 104, and the femur sensor device 106 can be couped to the pelvic bracket 110 for calibration. The method can include placing the navigation device 102 flat on the table. The method can include waiting for an audible beep. The beep can indicate that the user can proceed to the next calibration step.

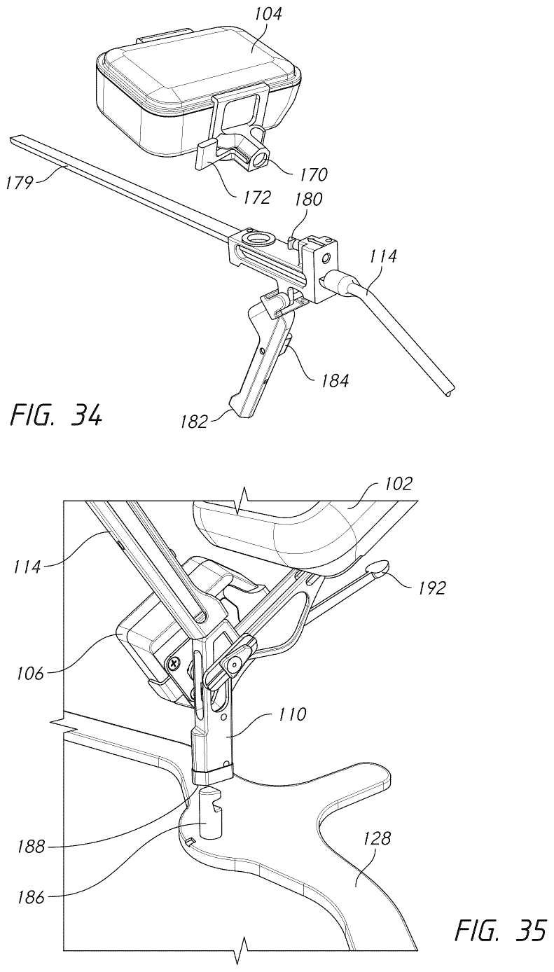

FIG. 34 is a view of the reference sensor device 104 and the registration probe 114. The registration probe 114 can have a range of motion relative to the pelvic bracket 110, when coupled thereto, to facilitate placement of the tip of the registration probe 114 in contact with a point. The registration probe 114 can contact one or more points on the pelvis for determining a reference frame. The registration probe 114 can contact a point on the femur for determining leg length and joint offset. The registration probe 114 can contact a point on the registration wand 132 for determining a center of rotation. The registration probe 114 can contact one or more points spaced apart from the attachment location of the fixation pins 156, 158.

The reference sensor device 104 can detect the orientation and rotation of the registration probe 114. The registration probe 114 can include a marking 179. The marking can indicate length or extension of the registration probe 114. The marking can be positioned on registration probe 114 beneath the reference sensor device 104 in use. The reference sensor device 104 can include a camera 181, shown in FIG. 33. The camera 181 of the reference sensor device 104 can capture an image of the marking 179 of the registration probe 114. The reference sensor device 104 and/or the navigation device 102 can determine the translational position of the registration probe 114 utilizing the captured image. In some embodiments, the reference sensor device 104 is capable of determining orientation, rotation, and position.

The method can include removing the reference sensor device 104 from the pelvic bracket 110. The method can include coupling the reference sensor device 104 to the registration probe 114. The registration probe 114 can include a coupler 180. The reference sensor device 104 can have the corresponding receiver 170 configured to couple to the coupler 180 of the registration probe 114. The user can depress the lever 172 on the reference sensor device 104 to engage the coupler 180 of the registration probe 114. The registration probe 114 can include a corresponding receiver 182. The corresponding receiver 182 of the registration probe 114 is configured to securely couple to the coupler 168 of the pelvic bracket 110. The user can depress a lever 184 on the registration probe 114 to engage the coupler 168 of the pelvic bracket 110.

FIG. 35 is a view of the probe calibration jig 128. The method can include coupling the registration probe 114 to the pelvic bracket 110. The reference sensor device 104 can be coupled to the registration probe 114. The navigation device 102 and the femur sensor device 106 can be couped to the pelvic bracket 110. The method can include coupling the pelvic bracket 110 to the probe calibration jig 128. The probe calibration jig 128 can include a coupler 186. The pelvic bracket 110 can include a corresponding receiver 188. The corresponding receiver 188 of the pelvic bracket 110 is configured to securely couple to the coupler 186 of the probe calibration jig 128. The method can include depressing a lever 192 on the back of the pelvic bracket 110. The method can include dropping the receiver 188 of the pelvic bracket 110 into place relative to the probe calibration jig 128. The method can include releasing the lever 192 when fully seated. The screen can provide instructions to the user to lock the probe calibration jig 128.

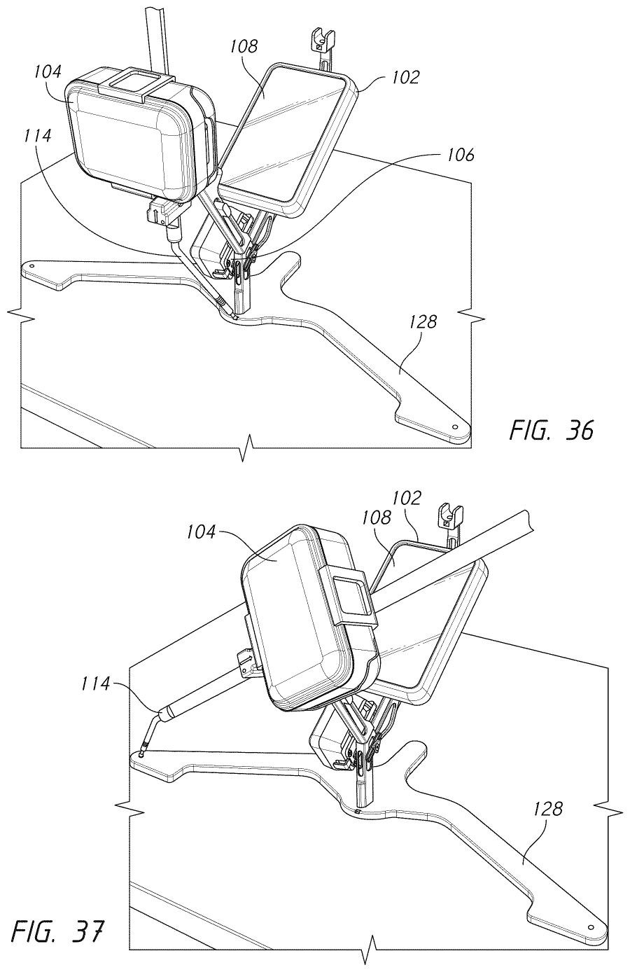

The method can include calibration using the probe calibration jig 128. FIG. 36 is a view of the navigation device 102, the reference sensor device 104, the femur sensor device 106, and the probe calibration jig 128. The user can place the tip of the registration probe 114 into a center point on the probe calibration jig 128. The user can interact with the graphical user interface 108 of the navigation device 102 to press register. The navigation device 102 can record the location when the tip of the registration probe 114 is placed in the center point.

FIG. 37 is a view of the navigation device 102, the reference sensor 104, the femur sensor device 106, and the probe calibration jig 128. The user can place the tip of the registration probe 114 into a left point on the probe calibration jig 128. The user can interact with the graphical user interface 108 of the navigation device 102 to press register. The navigation device 102 can record the location when the tip of the registration probe 114 is placed in the left point.

FIG. 38 is a view of the navigation device 102, the reference sensor 104, the femur sensor device 106, and the probe calibration jig 128. The user can place the tip of the registration probe 114 into a right point on the probe calibration jig 128. The user can interact with the graphical user interface 108 of the navigation device 102 to press register. The navigation device 102 can record the location when the tip of the registration probe 114 is placed in the right point.

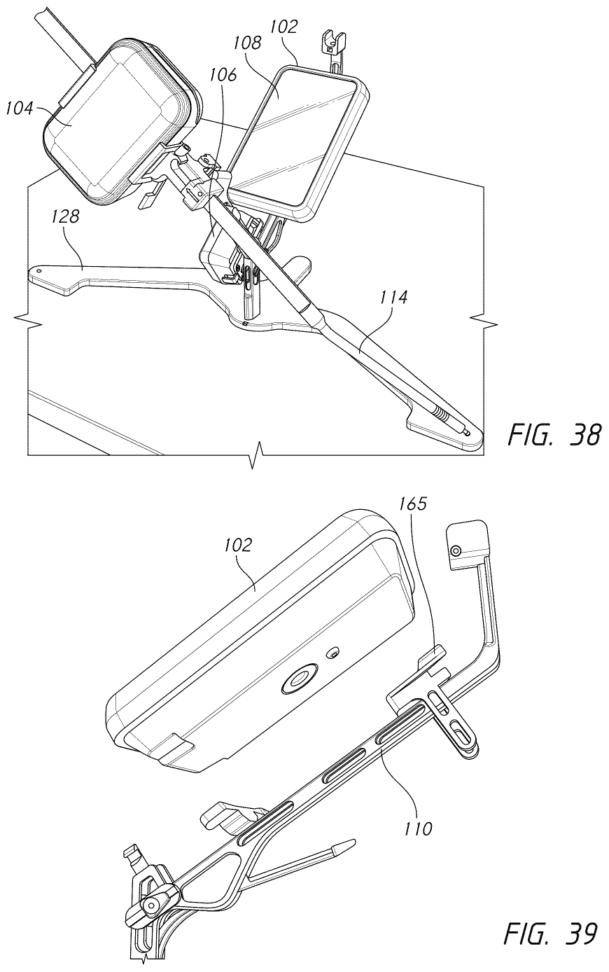

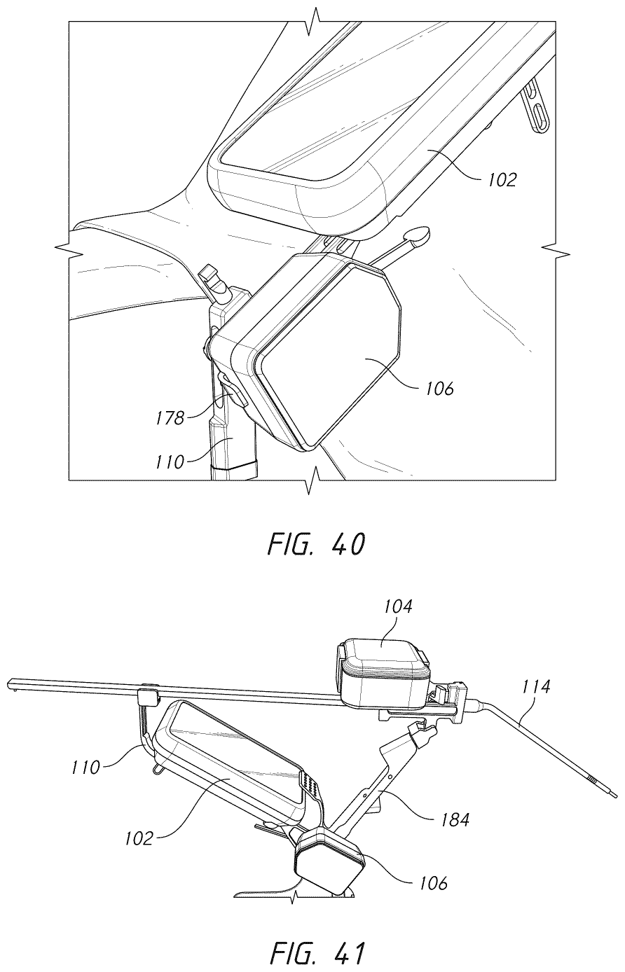

The method can include assembling the hip navigation system. FIG. 39 is a view of the navigation device 102. The user can attach the navigation device 102 to the pelvic bracket 110 via the magnetic interface of the magnetic section 165. FIG. 40 is a view of the navigation device 102 and the femur sensor device 106. The user can attach the femur sensor device 106 to the lateral side of the pelvic bracket 110 by depressing the lever 178 of the femur sensor device 106. The foot of the femur sensor device 106 should always be oriented towards the foot of the patient during all transitions throughout the procedure. The user can ensure that the femur sensor device 106 is fully attached and the lever 178 is fully engaged after transferring the femur sensor device 106 to a new location. FIG. 41 is a view of the navigation device 102, the reference sensor device 104, and the femur sensor device 106. The user can attach the registration probe 114 to the pelvic bracket 110 by depressing the lever 184 of the registration probe 114.

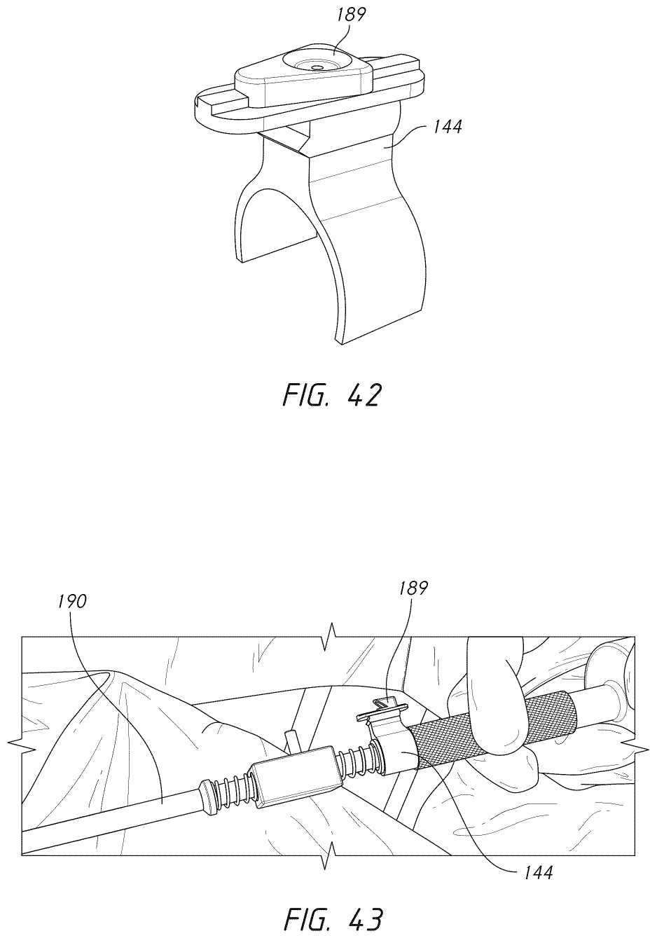

The method can include assembling the impactor. The kit can include one or more modular impactor adapter clips 144, 146 for use with implant impactors from various manufactures. The one or more impactor adapter clips 144, 146 can be configured to couple to offset impactors. The one or more impactor adapter clips 144, 146 can be configured to couple to straight impactors. FIG. 42 is a view of the impactor adapter clip 144. FIG. 43 is a view of the impactor adapter clip 144 and an impactor 190. The method can include assembling the impactor adapter clip 144 to a shaft of the impactor 190. The impactor adapter clip 144 can include an inner surface configured to mate with the shaft of the impactor 190. The impactor adapter clip 144 can be configured to clip onto the shaft of the impactor 190. The femur sensor device 106 can be coupled to the impactor adapter clip 144. The impactor adapter clip 144 can include a coupler 189. The femur sensor device 106 can have a corresponding receiver 176 configured to securely couple to the coupler 189 of the impactor adapter clip 144. The user can depress the lever 178 on the femur sensor device 106 to engage the coupler 189.

The method can include assembling instruments corresponding to the femoral fixation location. The method can include using the leg length and joint offset feature. The user can follow the appropriate steps that correspond with the previously selected femoral fixation location, either greater trochanter or distal thigh. For greater trochanter, the user should have available the fixation plate 150, the fixation inserter handle 118, the fixation screw 148, and the fixation tower 116, referring back to FIG. 9. The method can include assembling fixation inserter handle 118 and the fixation tower 116 to form an inserter. The method can include assembling the fixation plate 150 to the inserter. The method can include pressing the lever on the side of the inserter, then pushing the inserter down onto the fixation plate 150. The method can include releasing the lever to capture the fixation plate 150, then lift the fixation plate 150 out of the tray base 162. The inserter can lift the fixation plate 150 from the tray base 162 to avoid contacting the sharp spikes on the fixation plate 150.

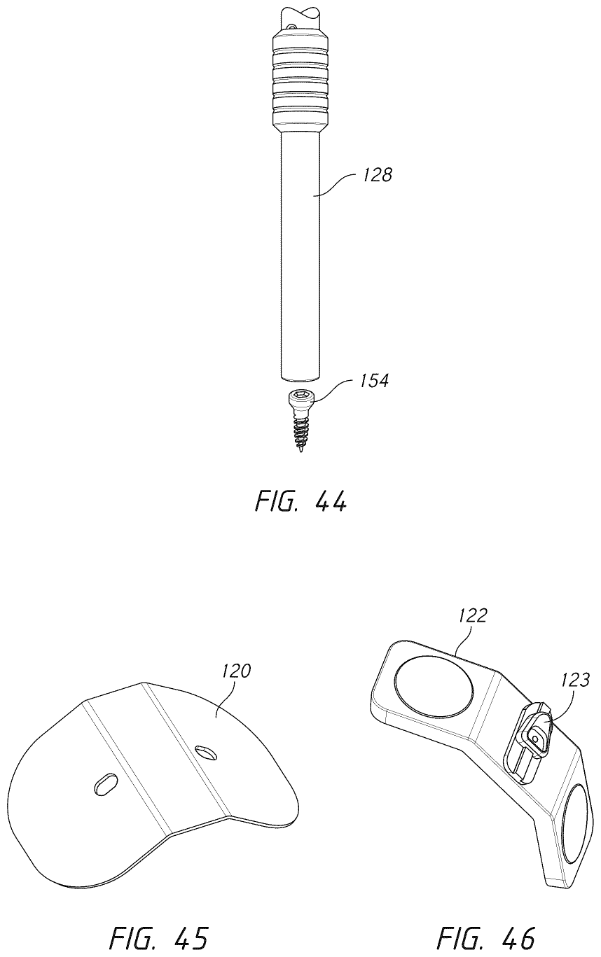

For distal thigh, the user should have available the thigh plate 120 and the thigh mount 122, referring back to FIG. 9. For distal thigh, the user should have available Coban™ and/or Ioban™ which can be supplied separately from the tray base 162. The method can include pressing the holding sleeve 126 onto the hex driver 124 with the smaller end closer to the handle. The method can include assembling the femur registration marker 154 to the holding sleeve 126. The method can include pinching the back of the holding sleeve 126 to extend the arms, then place the femur registration marker 154 into the tip of the hex driver 124. The method can include releasing the back of the holding sleeve 126 to grip the femur registration marker 154. The method can include an alternative fixation option including the screw 152. FIG. 44 is a view of a femur registration marker 154. FIG. 45 is a view of the thigh plate 120. FIG. 46 is a view of the thigh mount 122. The thigh mount 122 can include a coupler 123. The femur sensor device 106 can have a corresponding receiver 176 configured to securely couple to the coupler 123 of the thigh mount 122. The user can depress the lever 178 on the femur sensor device 106 to engage the thigh mount 122.

The method can include patient positioning and preparation. The patient is positioned supine on the operative table. The preparation can include draping such that the user can place pins fixation 156, 158 in the ipsilateral iliac crest. Other configurations are contemplated (e.g., one fixation pin, three fixation pins, four fixation pins, etc.). The fixation pins 156, 158 can be elongated structures. The fixation pins 156, 158 can be threaded. The method can include taping the pannus, which may allow for easier access to anatomic landmarks for registration. The preparation can including draping such that draping allows for the thigh plate 120 to be affixed to skin on the distal thigh if choosing the distal thigh fixation for leg length. Registration must be completed prior to femoral neck resection and dislocation.

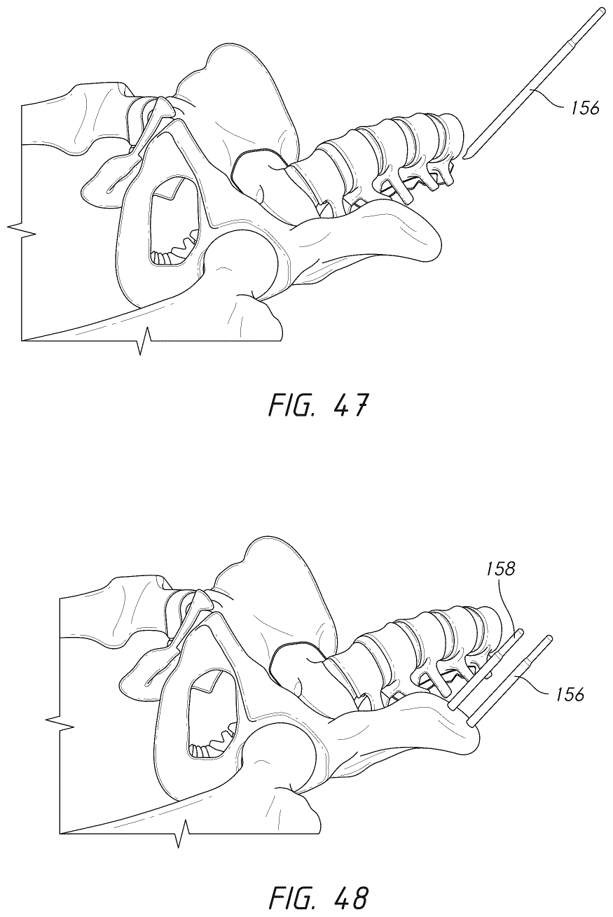

The method can include a solid and stable mount for attachment of the instrumentation to the pelvis. In some methods, all measurements and references are based off the initial registration process and any movements of the pelvic bracket 110 and the pelvic base 112 thereafter may produce inaccurate readouts. The method can include placing the two fixation pins 156, 158 in the ipsilateral iliac crest in a parallel fashion; similar to a pelvic external fixator. The fixation pins 156, 158 can enter the iliac wing at its most superior surface and travel between the tables of the iliac wing. FIG. 47 is a view of the fixation pin 156. FIG. 48 is a view of the fixation pins 156, 158. In some methods, the most anterior pin should be placed 2-4 cm posterior to the ASIS. In some methods, the optimal position for the fixation pins 156, 158 is to enter at approximately a 45-degree angle to enter into the thicker supra-acetabular bone along the anterior column. In some methods, fixation pins that are passed at an angle greater than 45 degrees are at risk for exiting the cortical bone where the ilium thins more posteriorly. The fixation pins 156, 158 can include a pointed tip to allow for secure placement while starting through the thicker outer cortical bone, which decreases the risk of pin slippage and aberrant placement. The user can feel for any significant resistance when passing the fixation pins 156, 158 to an appropriate depth to avoid perforating the cortical bone. In some methods, the fixation pins 156, 158 have the best stability if the fixation pins 156, 158 are placed at least half their length into the bone, which provides a stable construct with the pelvic base 112.

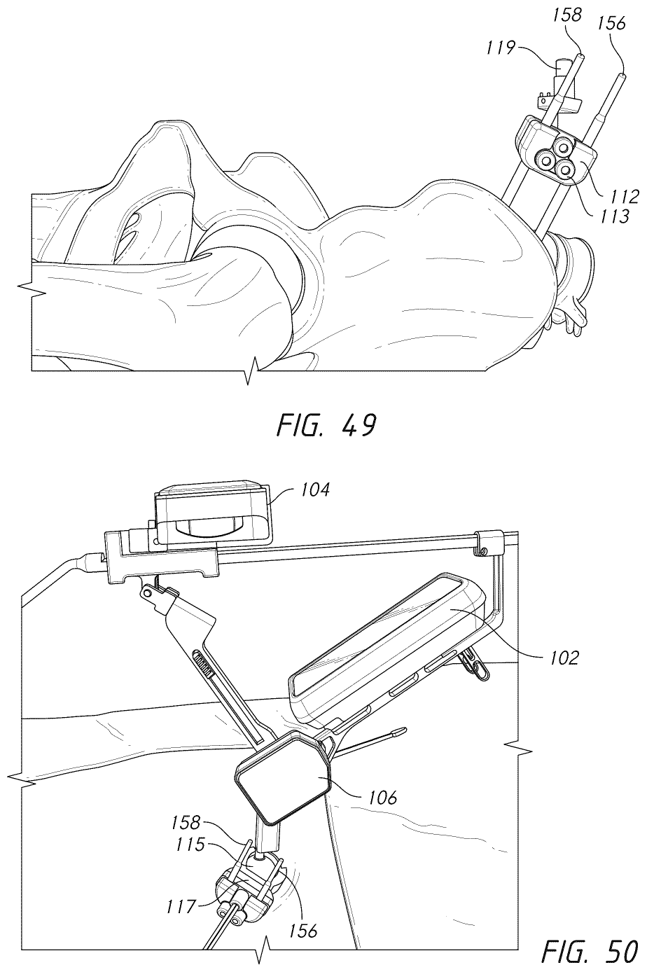

The method can include coupling the pelvic base 112. FIG. 49 is a view of the pelvic base 112. The pelvic base 112 can function as a clamp with the fixation pins 156, 158. The pelvic base 112 can include one or more screws 113. Rotation of the screws 113 can cause the pelvic base 112 to clamp onto the fixation pins 156, 158. Prior to assembling the pelvic base 112 onto the fixation pins 156, 158, the user can ensure that the bottom screws 113 of the pelvic base 112 are loose. This will allow the fixation pins 156, 158 to fit easily inside channels of the pelvic base 112. The method can include assembling the pelvic base 112 onto the fixation pins 156, 158 approximately 1 cm above skin. The method can include tightening the bottom two screws 113 of the pelvic base 112. The method can include supporting the pelvic base 112 with one hand while tightening the screws 113 of the pelvic base 112 to minimize torque applied to the fixation pins 156, 158 while tightening the screws 113 of the pelvic base 112.

The pelvic base 112 can include a ball joint 115. The method can include moving the ball joint relative to the pelvis. The method can include provisionally positioning the ball joint 115 of the pelvic base 112 such that a registration probe divot 117 of the pelvic base 112 points towards the patient's feet and a coupler 119 of the pelvic base 112 points as vertical as possible. The method can include tightening the top screws 113 to lock the ball joint 115, such that the navigation device 102 is angled at approximately 45 degrees to the floor. The method can include tightening the screws 113 of the pelvic base 112. FIG. 50 is a view of the navigation device 102, the reference sensor device 104, the femur sensor device 106, the pelvic bracket 110, and the pelvic base 112.

The method can include femoral fixation preparation. In some methods, prior to resecting the femoral head, the user can select a femoral fixation location, either the distal femur or greater trochanter. The method can include following the appropriate steps.

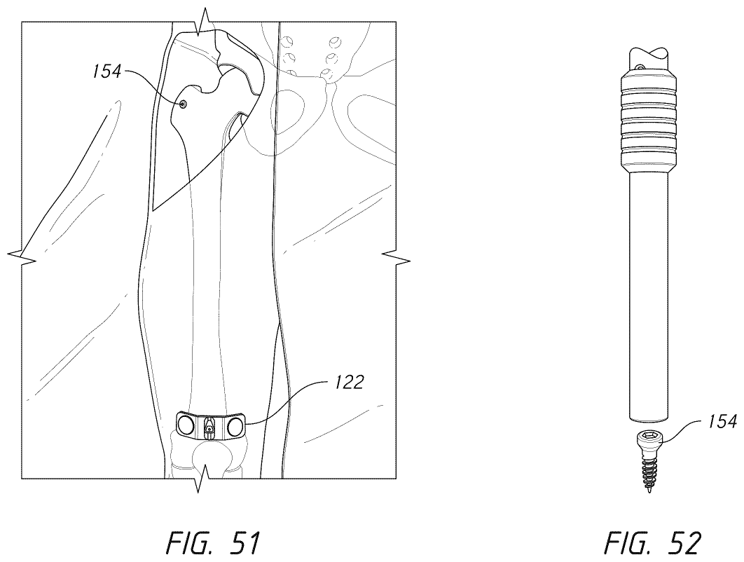

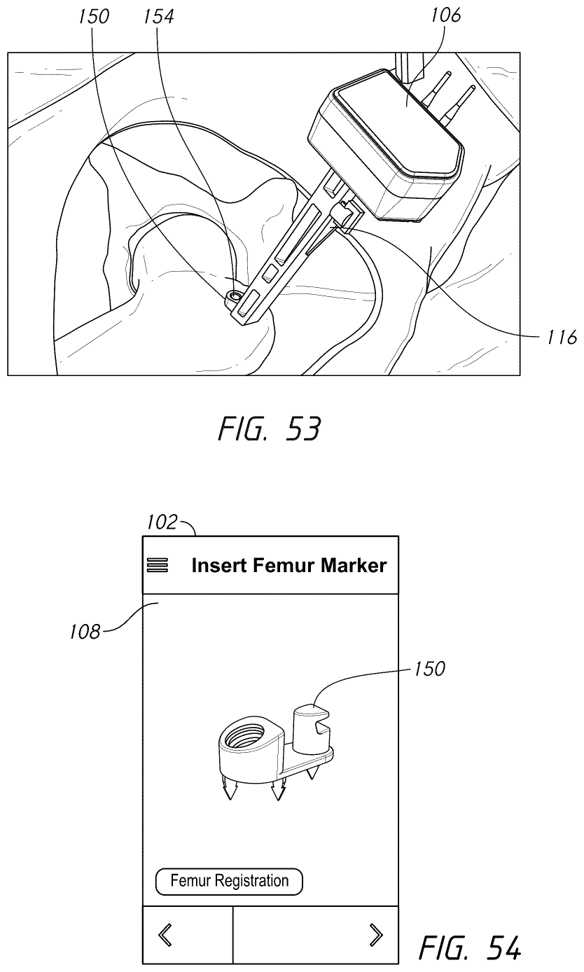

The method can include femoral fixation set up on the distal thigh. The method can include wrapping the distal thigh in Coban™M, or similar self-adherent wrap with latex, to minimize skin movement. The method can include placing the thigh plate 120 onto the Coban™ wrapped skin of the distal thigh. The user can confirm that the position of the thigh plate 120 is approximately 4-5cm superior to the superior pole of the patella. The user can confirm that the position of the thigh plate 120 is on Coban™ wrapped skin, not on a drape. The method can include securing the thigh plate 120 to the distal thigh using a small strip of Ioban™, or similar antimicrobial incise drape. The method can include placing one horizontal and one vertical strip of Ioban™ on the thigh plate 120. The method can include selecting the femur registration marker 154 and assembling the femur registration marker 154 onto the hex driver 124 using the holding sleeve 126. The method can include inserting the femur registration marker 154 into the anterior greater trochanter. The user can ensure that the femur registration marker 154 is in hard bone. The user can ensure that the femur registration marker 154 will not interfere with femoral broaching. The user can ensure that the femur registration marker 154 can be reached by the registration probe 114. The method can include seating the femur registration marker 154 by using a small mallet to gently tap the top of the hex driver 124. The method can include remove the hex driver 124 by pinching the back of the holding sleeve 126 and pulling up. FIG. 51 is a view of the thigh mount 122 and the femur registration marker 154. FIG. 52 is a view of the femur registration marker 154.

The method can include femoral fixation set up on the greater trochanter. The method can include identifying a location to seat the fixation plate 150. The user can ensure that the fixation plate 150 is in hard bone. The user can ensure that the fixation plate 150 is out of the way of broaching. The user can ensure the orientation of fixation plate 150 such that the head of the femur registration marker 154 may be easily accessed. The method can include seating the fixation plate 150 by using a small mallet to gently tap the top of the fixation inserter handle 118. The method can include selecting the femur registration marker 154, either a non-locking screw or a locking screw. The method can include inserting the femur registration marker 154 into the guide. The method can include using the hex driver 124 to insert the femur registration marker 154 into bone. The user can avoid compromising fixation by delivering too much torque to the femur registration marker 154. The user can consider releasing the vastus lateralis in order to secure bony fixation. The method can include attaching the fixation tower 116 to the fixation plate 150. FIG. 53 is a view of the fixation tower 116 and the femur sensor device 106. FIG. 54 is a view of a screen of the graphical user interface 108 of the navigation device 102. The graphical user interface 108 can provide instructions for placement of the fixation plate 150. In some methods, the user is advised not to complete femoral neck resection and dislocation prior to completing all registrations.

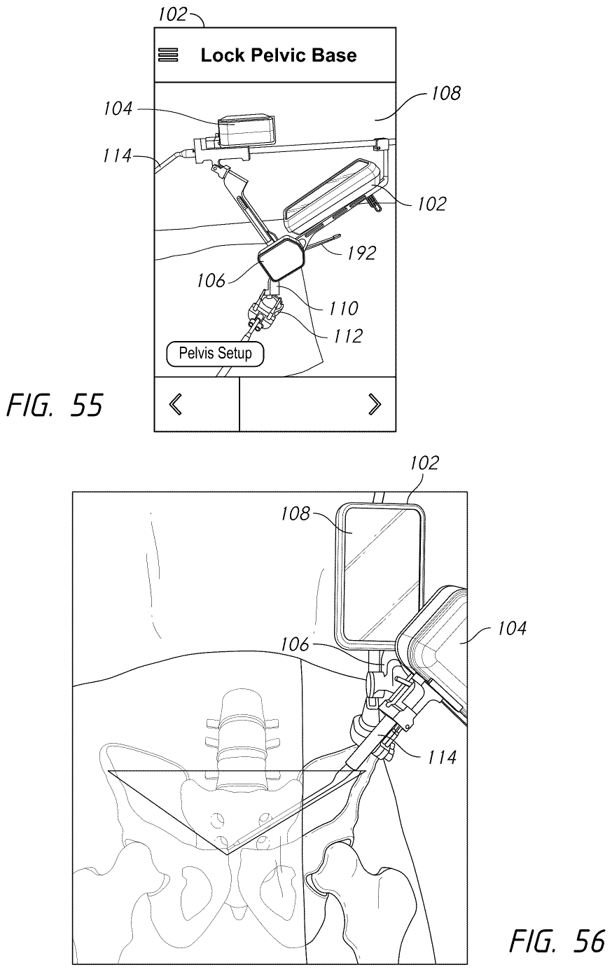

The method can include registration. The method can include assembling the pelvic bracket 110 to the pelvic base 112. The method can include depressing the lever 192 on the back of the pelvic bracket 110. The method can include dropping the receiver 188 of the pelvic bracket 110 into place relative to the coupler 119 the pelvic base 112, shown in FIG. 49. The method can include releasing the lever 192 when fully seated. The navigation device 102, the registration probe 114, the reference sensor device 104, and the femur sensor device 106 can be coupled to the pelvic bracket 110. FIG. 55 is a view of a screen of the graphical user interface 108 of the navigation device 102. The screen can provide instructions to the user to lock the pelvic bracket 110 to the pelvic base 112.

The method can include registering the patient's anterior pelvic plane (APP). The user can palpate each point. The method can include positioning the tip of the registration probe 114 in contact with a point. The method can include pressing register on the graphical user interface 108 of the navigation device 102. The method can include positioning the tip of the registration probe 114 in contact with the Ipsilateral ASIS. The method can include pressing register to register the Ipsilateral ASIS on the graphical user interface 108 of the navigation device 102. The method can include positioning the tip of the registration probe 114 in contact with the Contralateral ASIS. The method can include pressing register to register the Contralateral ASIS on the graphical user interface 108 of the navigation device 102. The method can include positioning the tip of the registration probe 114 in contact with the Pubis. The user can register the higher side of the Pubis. The method can include pressing register to register the Pubis on the graphical user interface 108 of the navigation device 102. The method can include positioning the tip of the registration probe 114 into contact with three points sequentially. The method can include positioning the tip of the registration probe 114 to register three points sequentially. The method can include positioning the tip of the registration probe 114 to register one or more anatomical landmarks. The user can use similar force when palpating each point. The user can ensure the operating table is parallel to the floor. The user can ensure that the patient's foot is neutral with no traction. The user can ensure that the sagittal plane for cup navigation is set perpendicular to the registered trans-ASIS line. FIG. 56 is a view of the navigation device 102, the reference sensor device 104, the femur sensor device 106, and the registration probe 114.

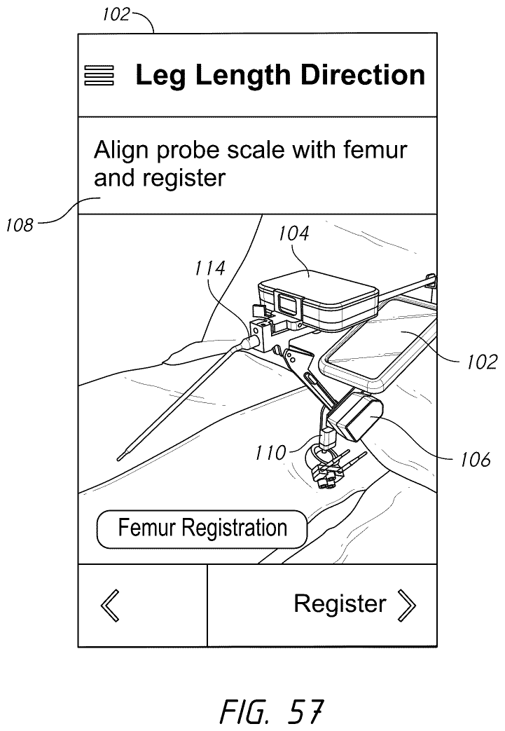

The method can include registering leg length direction. The method can include registering leg length direction by aligning the barrel of the registration probe 114 with the long axis of the leg. The method can include pressing register to register the leg length direction on the graphical user interface 108 of the navigation device 102. FIG. 57 is a view of a screen of the graphical user interface 108 of the navigation device 102. The method can include aligning the marking 179 of the registration probe 114 with the patient's femur. The method can include registration. The user can ensure the femur sensor device 106 is always attached to the same side of the pelvic bracket 110 as during initial registration. For instance, if the femur sensor device 106 is on the left side of the pelvic bracket 110 during initial registration, then return the femur sensor device 106 to the left side during cup navigation, leg length measurements, and joint offset measurement. For instance, if the femur sensor device 106 is on the right side of the pelvic bracket 110 during initial registration, then return the femur sensor device 106 to the right side during cup navigation, leg length measurements, and joint offset measurement.

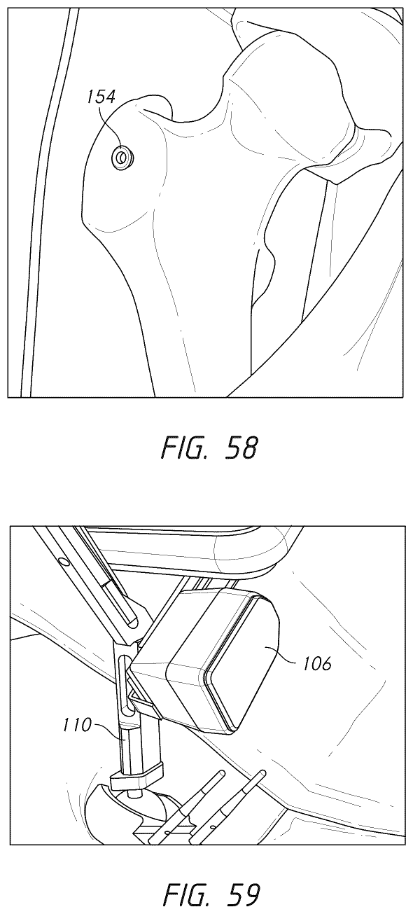

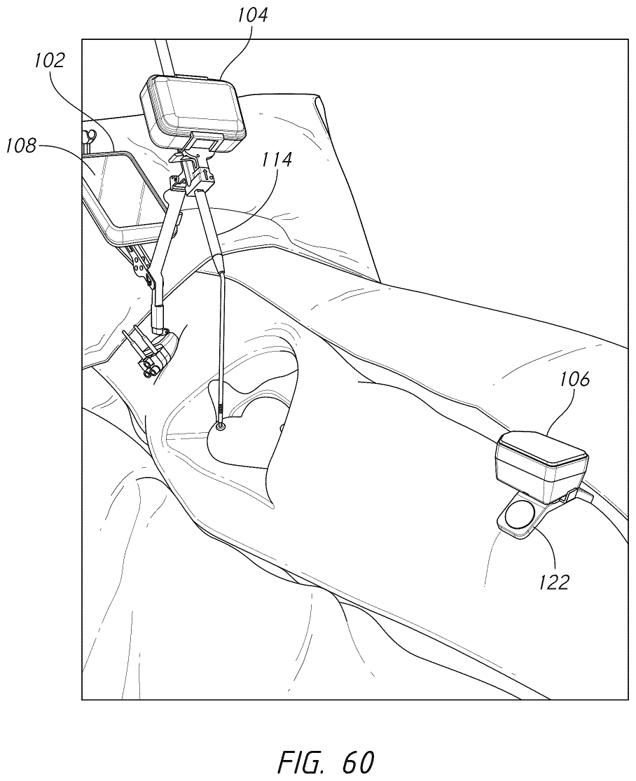

The method can include femur registration. If the user is using the leg length and offset feature, then complete the femur registration for either the distal thigh or greater trochanter based on the user's earlier selection. For the distal thigh, the user can visually locate the femur registration marker 154 on the greater trochanter. FIG. 58 is a view of the femur registration marker 154. The method can include transferring the femur sensor device 106 from the pelvic bracket 110 to the thigh mount 122. The user should ensure that the foot of the femur sensor device 106 should always be oriented towards the foot of the patient during all transitions throughout the procedure. FIG. 59 is a view of the femur sensor device 106 mounted on the pelvic bracket 110. FIG. 60 is a view of the navigation device 102, the reference sensor device 104, the femur sensor device 106, and the registration probe 114. The femur sensor device 106 is mounted on the thigh mount 122. The method can include inserting the registration probe 114 into the head of the femur registration marker 154. The method can include pressing register to register the femur registration marker 154 on the graphical user interface 108 of the navigation device 102. The method can include removing the femur sensor device 106 from the thigh mount 122. The method can include removing the thigh mount 122, while leaving the thigh plate 120. The method can include removing the pelvic bracket 110. The method can include proceeding with standard acetabular bone preparation. FIG. 61 is a view of the femur sensor device 106 while coupled to the thigh mount 122.

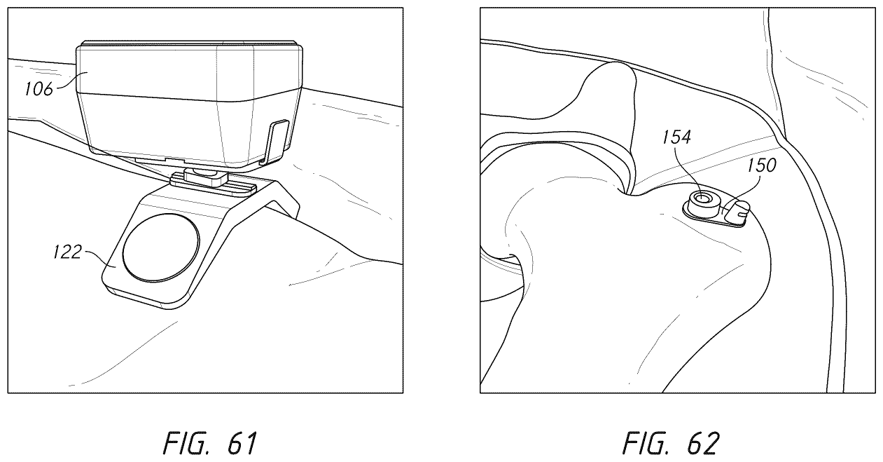

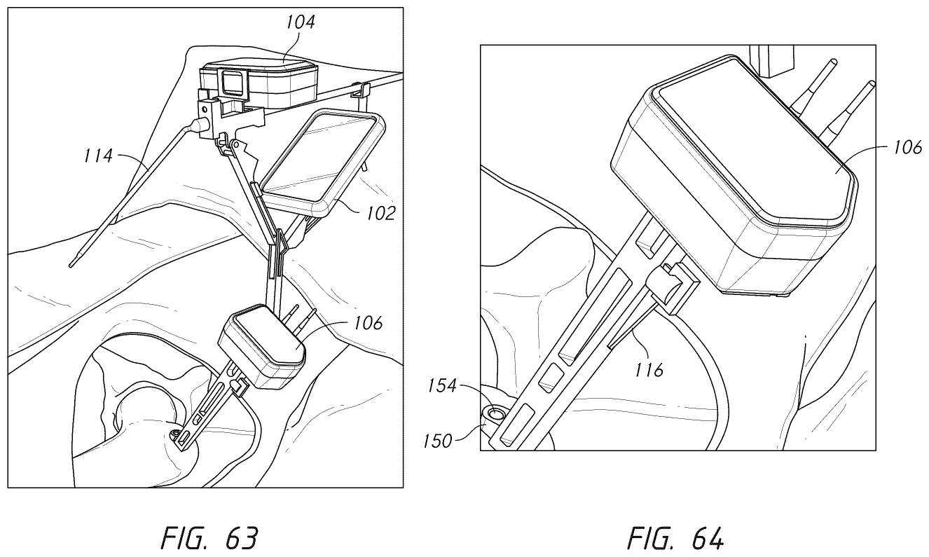

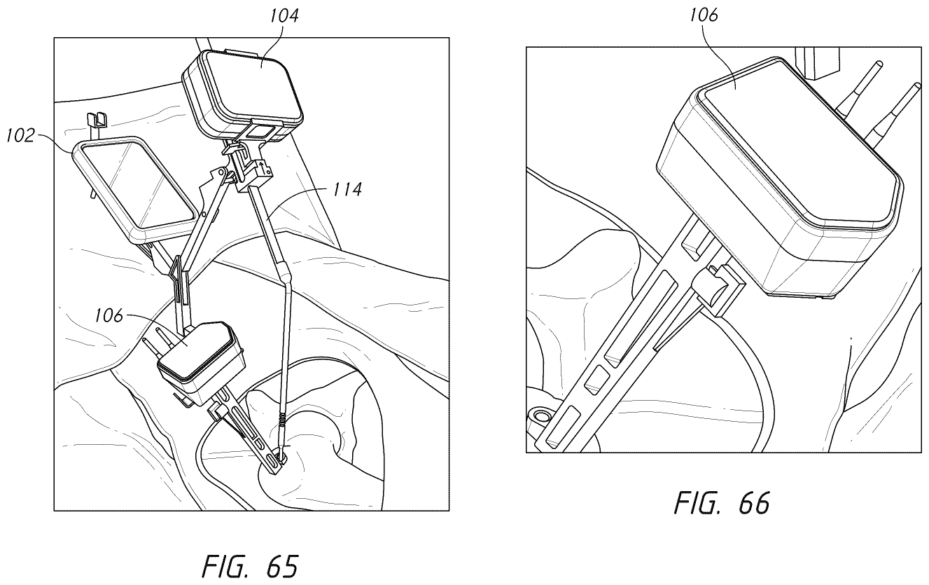

For the greater trochanter, the user can visually locate the fixation plate 150 and the femur registration marker 154 on the greater trochanter. The user can hold the fixation tower 116. FIG. 62 is a view of the fixation plate 150. The method can include transferring the femur sensor device 106 from the pelvic bracket 110 to the fixation tower 116. The method can include coupling the fixation tower 116 with the attached femur sensor device 106 to the fixation plate 150. FIG. 63 is a view of the navigation device 102, the reference sensor device 104, the femur sensor device 106, and the registration probe 114. FIG. 64 is a view of the femur sensor device 106. The femur sensor device 106 is mounted on the fixation tower 116. In some methods, the femur sensor device 106 is mounted on the fixation tower 116 before the fixation tower 116 is coupled to the fixation plate 150. The femur registration marker 154 is accessible to the registration probe 114 when the fixation tower 116 is coupled to the fixation plate 150. FIG. 65 is a view of the navigation device 102, the reference sensor device 104, the femur sensor device 106, and the registration probe 114. FIG. 66 is a view of the femur sensor device 106. The method can include inserting the registration probe 114 into the head of the femur registration marker 154. The method can include pressing register to register the femur registration marker 154 on the graphical user interface 108 of the navigation device 102. The method can include removing the femur sensor device 106. The method can include removing the fixation tower 116. The method can include removing the pelvic bracket 110. The method can include proceeding with standard acetabular bone preparation. After completing all initial registrations, then the user can proceed with femoral neck resection, dislocation, and manual cup reaming.

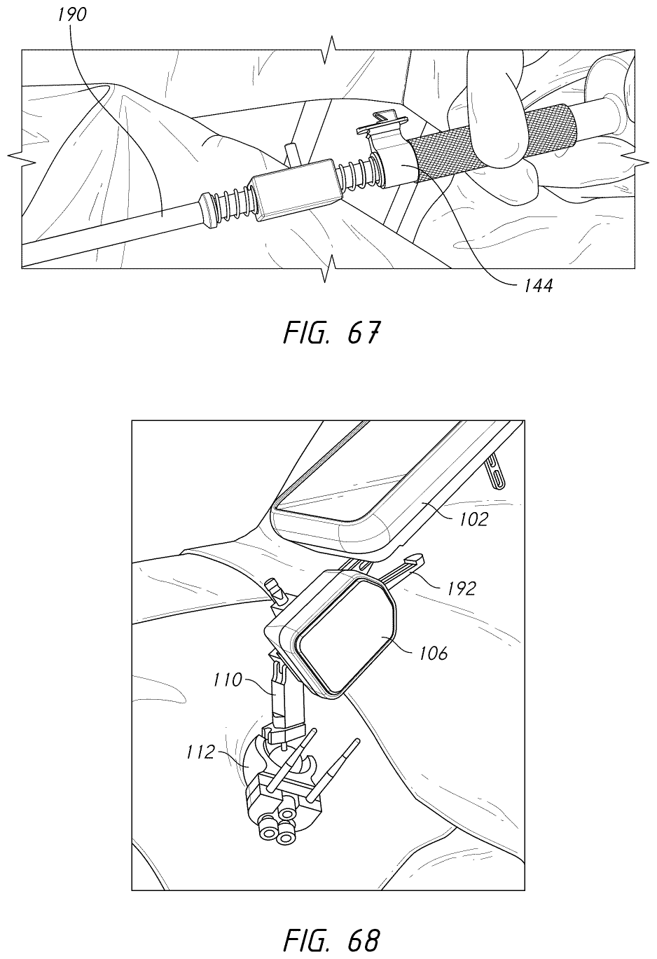

The method can include cup placement. The method can include trial assist. The method can include setting up cup navigation. The method can include attaching a trial cup to the impactor 190. The method can include attaching a cup to the impactor 190. The user can ensure that an appropriate impactor adapter clip 144, 146 is secured to the impactor 190 in the correct orientation. FIG. 67 is a view of the impactor adapter clip 144 and the impactor 190.

The method can include assembling the navigation device 102 and the femur sensor 106 onto the pelvic bracket 110. The method can include the pelvic bracket 110 onto the pelvic base 112. The method can include depressing the lever 192 on the back of the pelvic bracket 110. The method can include dropping the receiver 188 of the pelvic bracket 110 into place relative to the pelvic base 112. The method can include releasing the lever 192 when fully seated. The reference probe 114 and the reference sensor device 104 are not needed for this step. FIG. 68 is a view of the navigation device 102 and the femur sensor device 106.

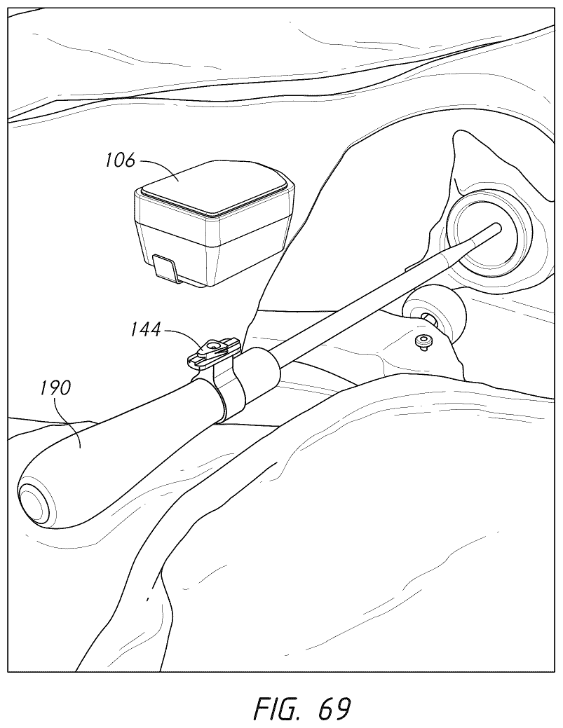

The method can include provisionally inserting the impactor 190 in the acetabulum. The user can ensure the impactor adapter clip 144 is oriented facing up such that the impactor adapter clip 144 may accept the femur sensor 106 transfer. FIG. 69 is a view of the femur sensor device 106, the impactor adapter clip 144, and the impactor 190.

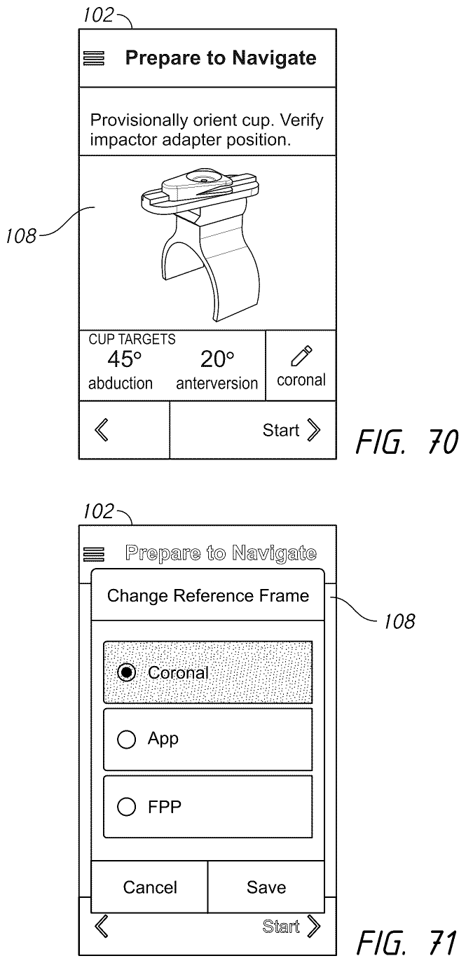

The method can include preparing to navigate. The method can include tapping start on the graphical user interface 108 of the navigation unit 102. The method can include holding steady until the navigation screen populates. The method can include provisionally orienting the cup. The method can include verifying the position of the impactor adapter clip 144. FIG. 70 is a view of a screen of the graphical user interface 108 of the navigation device 102. The screen can display the target cup angles. To change reference frames, the method can include tapping the button, then selecting the desired reference frame. The screen can display the selected reference frame. In some methods, the functional pelvic plane will only be available to be selected if a pelvic tilt value is entered during initial set up. FIG. 71 is a view of a screen of the graphical user interface 108 of the navigation device 102. The screen can display the available reference frames for selection. After selecting the button to change reference frames, this screen can populate for selection of desired reference frame.

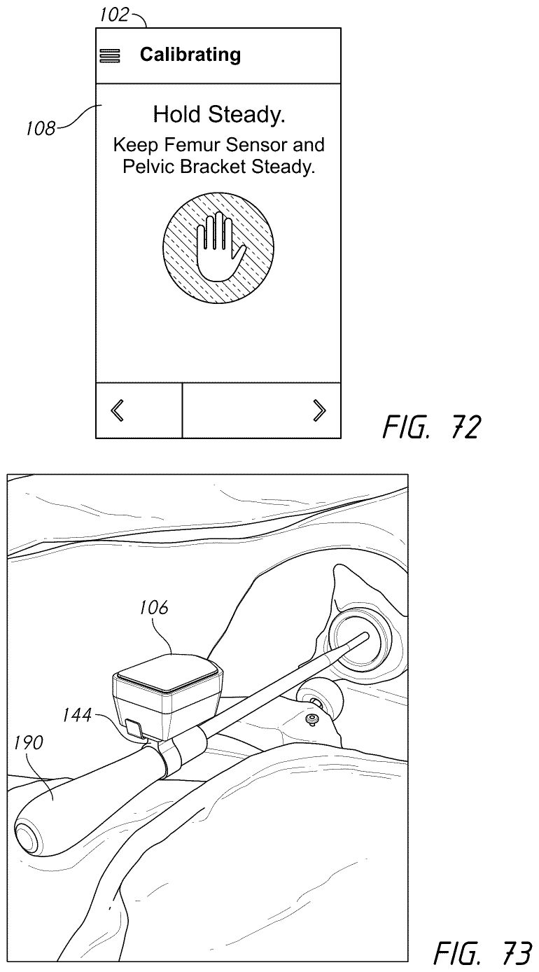

The method can include cup navigation. The navigation unit 102 must be stable during calibration process. The user can be advised not to touch the pelvic bracket 110 or pelvis during this step. The user can be advised not to touch the femur sensor 106 during this step. FIG. 72 is a view of a screen of the graphical user interface 108 of the navigation device 102. The graphical user interface 108 of the navigation device 102 can display an instruction. The graphical user interface 108 of the navigation device 102 can display a warning to the user.

The method can include removing the femur sensor device 106. While holding the impactor 190 steady at the desired orientation, the user can remove the femur sensor device 106 from the impactor 190. The user can remove the pelvic bracket 110 from the pelvic base 112. The user can impact the trial cup. The user can impact the cup. The user can be advised to not impact with the femur sensor device 106 attached to the impactor 190. The user can be advised to not impact with the pelvic bracket 110 attached to the pelvic base 112. FIG. 73 is a view of the femur sensor device 106, the impactor adapter clip 144, and the impactor 190.

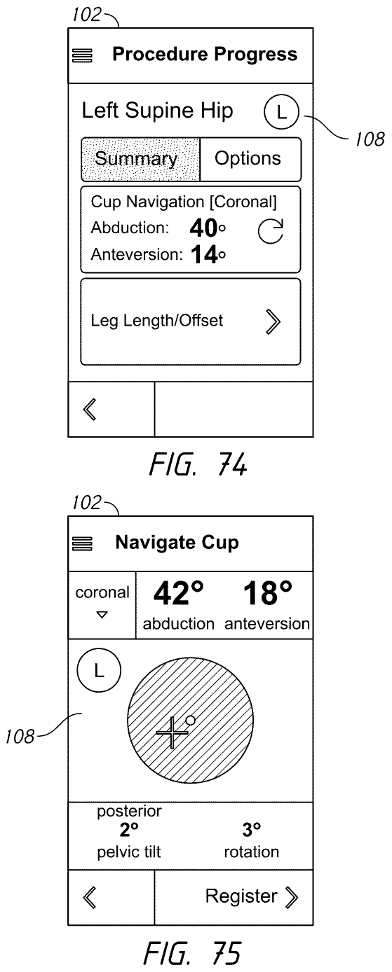

The method can include completing cup navigation. Upon completing cup navigation, the graphical user interface 108 of the navigation device 102 can display a summary of the cup placement. At this point, the user may either repeat cup navigation or proceed to leg length and offset calculations. FIG. 74 is a view of a screen of the graphical user interface 108 of the navigation device 102. The screen can provide a summary of the cup navigation. The screen can provide a summary of navigated abduction and anteversion angles. The screen can provide a summary of the reference frame used for the navigated angles. The method can include tapping the button to proceed to leg length and joint offset measurements.

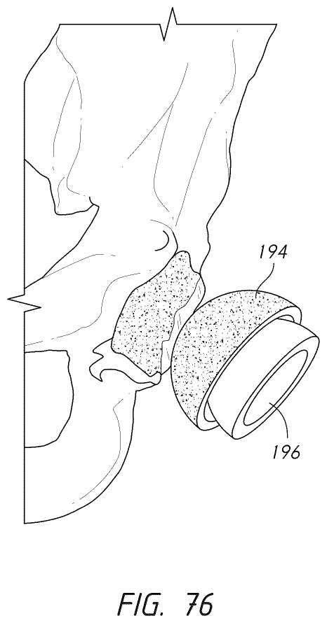

The method can include adjusting the cup orientation. Using the cup angles displayed on the graphical user interface 108 of the navigation device 102, the user may adjust the cup orientation as desired. Once the navigation screen populates, the user can transfer the femur sensor device 106 to the impactor adapter clip 144 coupled to the impactor 190. The angles displayed on the screen are relative to the reference frame selected at the beginning of the procedure. The method can include viewing angles relative to additional reference frames. The method can include viewing cup angles relative to additional reference frames by tapping the button in the top left corner to expand the window. The user can select an alternative reference frame. FIG. 75 is a view of a screen of the graphical user interface 108 of the navigation device 102. The screen can provide the ability to select alternative reference frames. The screen can provide live abduction and anteversion values.

The graphical user interface 108 of the navigation device 102 can provide a target visualization. The target visualization can be based on target angles inputted to the navigation device 102. The center of the target can indicate when the impactor 190 is navigated to target abduction and anteversion angles. The target can provide a visual representation of the orientation of the impactor 190 relative to the target values. The graphical user interface 108 of the navigation device 102 can provide a measurement of pelvic tilt. The graphical user interface 108 of the navigation device 102 can provide a measurement of pelvic rotation. The method can include saving navigation values. The method can include selecting register when the cup is in the desired orientation to save navigation values for later reference. In some methods, the navigation unit 102 will allow for 40 seconds of live navigation before the live measurement is stopped. The method can include repeating live measurements. The method can include restarting the process and resume live navigation.

The method can include measuring changes in leg length. The method can include measuring changes in joint offset. The method can include additional steps for users using leg length and joint offset measurements. The method can include removing the impactor 190 from a cup 194. The method can include inserting the desired liner 196 into the cup 194. FIG. 76 is a view of the cup 194 and a liner 196.

The method can include wand registration. The method can include selecting the appropriate size of liner registration trial. The liner registration trial mates with the implanted liner 196. The hip navigation system 100 can include the first liner registration trial 134. The first liner registration trial 134 can have a dimension of 28 mm. The hip navigation system 100 can include the second liner registration trial 136. The second liner registration trial 136 can have a dimension of 32 mm. The hip navigation system 100 can include the third liner registration trial 138. The third liner registration trial 138 can have a dimension of 36 mm. The hip navigation system 100 can include the fourth liner registration trial 140. The fourth liner registration trial 140 can have a dimension of 40 mm. The hip navigation system 100 can include the fifth liner registration trial 142. The fifth liner registration trial 142 can have a dimension of 44 mm. The hip navigation system 100 can include more than one liner registration trial. The hip navigation system 100 can include various sized liner registration trials to correspond with liners from various manufacturers. The method can include coupling one of the liner registration trials 134, 136, 138, 140, 142 to the registration wand 132.

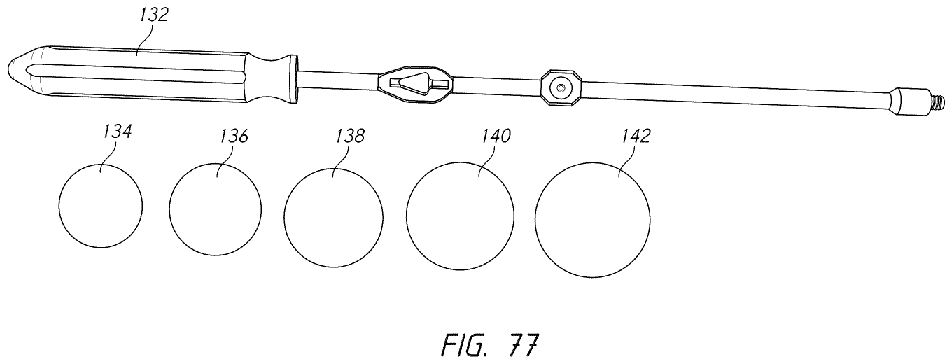

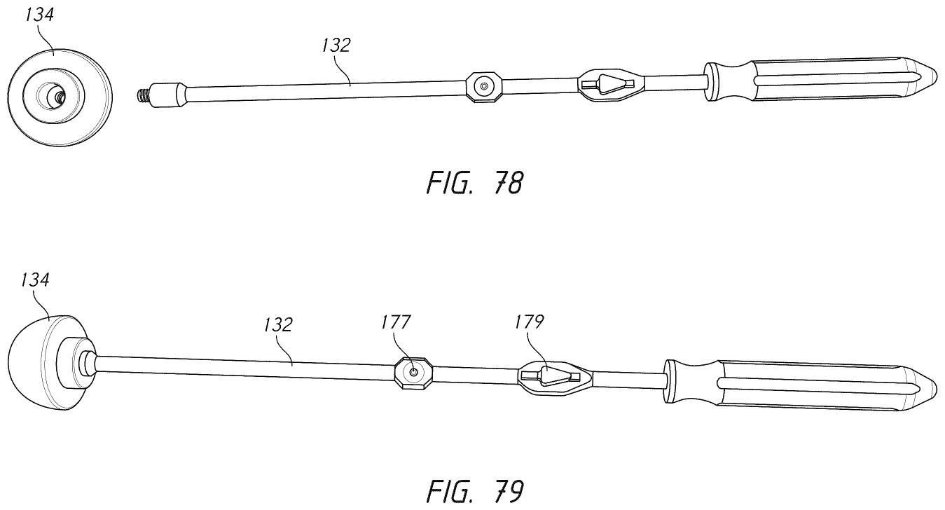

FIG. 77 is a view of the registration wand 132. The user can avoid cross-threading, as the liner registration trial 134, 136, 138, 140, 142 must be fully screwed onto the registration wand 132 to improve accuracy. The registration wand 132 and the liner registration trial 134, 136, 138, 140, 142 are used only for leg length and joint offset measurements. The method can include selecting the liner registration trial that corresponds with the chosen liner. The method can include assembling the selected liner registration trial 134, 136, 138, 140, 142 onto the registration wand 132. FIG. 78 is a view of the registration wand 132 and the liner registration trial 134. The method can include confirming the liner registration trial is fully seated. The user can be advised that incomplete or inappropriate seating of the liner registration trial may result in reduced accuracy. FIG. 79 is a view of the registration wand 132 and the liner registration trial 134. The registration wand 132 can include a divot 177. The registration wand 132 can include a coupler 179. The femur sensor device 106 can have the corresponding receiver 176 configured to securely couple to the coupler 179 of the registration wand 132. The user can depress the lever 178 on the femur sensor device 106 to engage the coupler 179 of the registration wand 132.

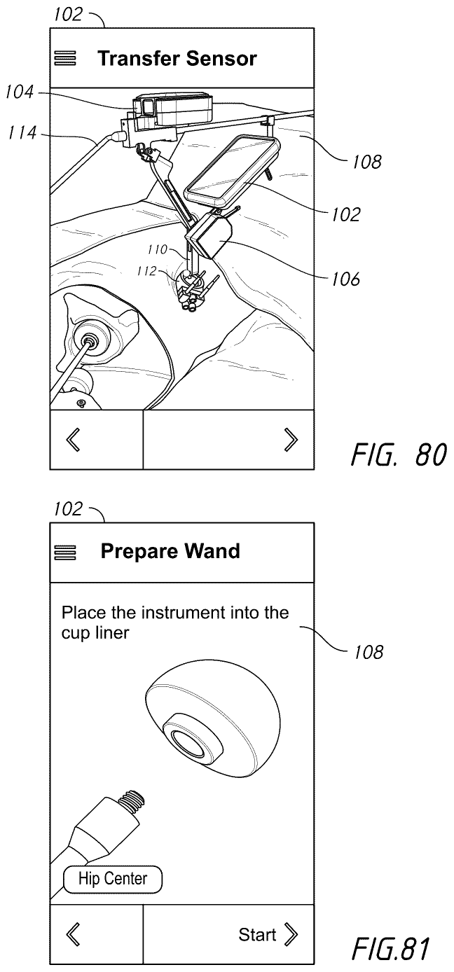

The method can include completing wand registration. The method can include returning the femur sensor device 106 to the pelvic bracket 110. The method can include assembling the registration probe 114 with the reference sensor device 104 onto the pelvic bracket 110. The method can include securing the pelvic bracket 110 with the femur sensor device 106, the registration probe 114, and the reference sensor device 104 to the pelvic base 112. FIG. 80 is a view of a screen of the graphical user interface 108 of the navigation device 102. The screen can instruct the user to transfer the femur sensor device 106. The screen can illustrate the desired assembly of the hip navigation system 100.

The method can include preparing the registration wand 132. The method can include tapping the start button on the graphical user interface 108 of the navigation device 102. The method can include placing the registration wand 132 into the liner registration trial 134. The user can hold steady during the red stoplight displayed on the screen. FIG. 81 is a view of a screen of the graphical user interface 108 of the navigation device 102.

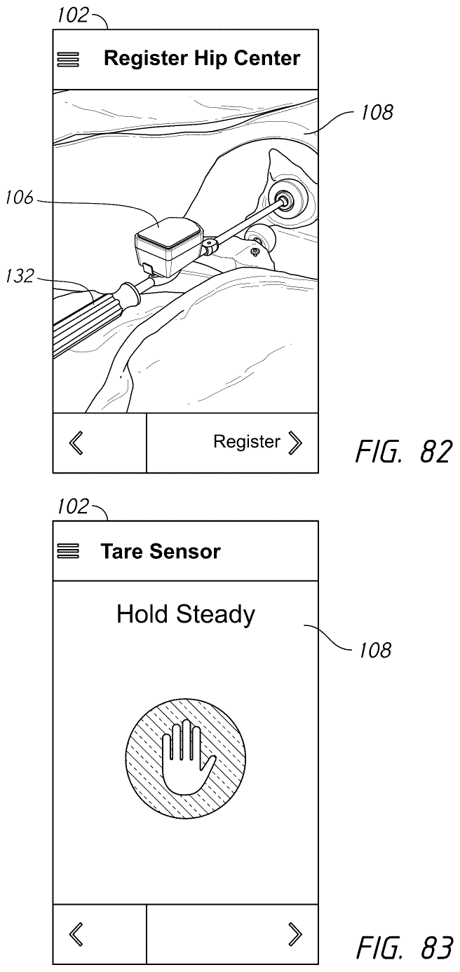

The method can include registering hip center. The method can include fully inserting the liner registration trial 134 of the registration wand 132 into the liner 196 of the cup 194. The graphical user interface 108 of the navigation device 102 can instruct the user to hold the registration wand 132 steady. The graphical user interface 108 of the navigation device 102 can display a graphic, such as a red stoplight. The user can hold steady during the red stoplight displayed on the screen. Once the hold steady graphic changes, the method can include transferring the femur sensor device 106 to the registration wand 132. In some methods, the wand registration must be completed within 30 seconds. If not completed, the method can include pressing the back arrow to restart the registration. FIG. 82 is a view of a screen of the graphical user interface 108 of the navigation device 102. The screen can provide an illustration of the method to register hip center with the registration wand 132. FIG. 83 is a view of a screen of the graphical user interface 108 of the navigation device 102. The screen can instruct the user to hold the registration wand 132 steady. The screen can instruct the user to tare the femur sensor device 106. In some methods, the registration wand 132 may be in any orientation, so long as the liner registration trial 134 is fully seated into the liner 196 concentrically. In some methods, the liner registration trial 134 is positioned concentric with the liner 196. In some methods, the liner registration trial 134 is positioned concentric with the cup 194. In some methods, only standard sized liners 196 can be used corresponding to 28 mm, 32 mm, 36 mm, 40 mm, and 44 mm. In some methods, if using any liner not corresponding to the sizes provided, the results could be less accurate. In some methods, the liner 196 is not a dual mobility liner.

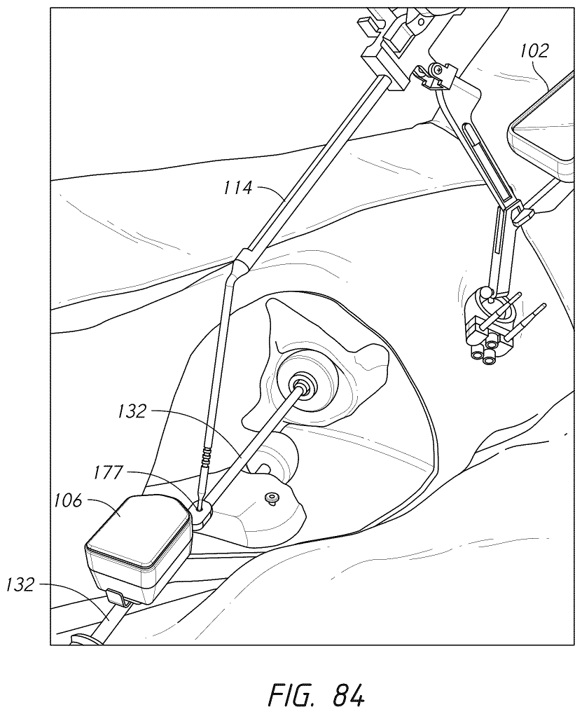

The method can include registering the registration wand 132. The method can include registering the location of the registration wand 132. The method can include registering the registration wand by inserting the tip of the registration probe 114 into a divot 177 on the registration wand 132. The method can include holding registration probe 114 steady. The method can include pressing register on the graphical user interface 108 of the navigation device 102 to register the registration wand 132. FIG. 84 is a view of the femur sensor device 106, the registration probe 114, and the registration wand 132. In some methods, if the user changes the liner 196 after initial registration and the new liner 196 changes lateralization, then the user must repeat hip center registration using the registration wand 132. The method can include removing the registration wand 132 with the liner registration trial 134. The method can include standard femoral bone preparation and trial reduction.

The method can include measuring leg length and joint offset. Once the desired trial cup and/or implants are in place and the hip is reduced, the method can include positioning the femur near the same position as initial registration. The user can ensure that the leg is oriented near the same position as initial registration including neutral foot rotation with no traction.

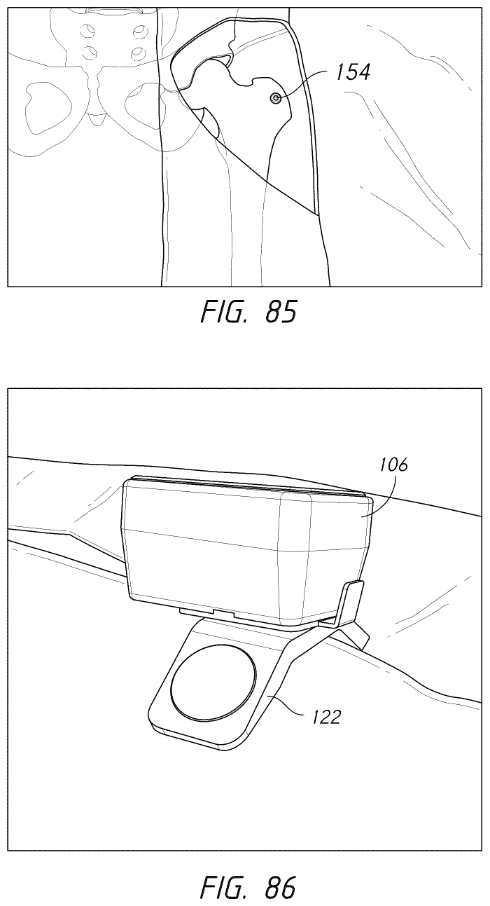

For the distal thigh, the user can visually locate the femur registration marker 154 on the greater trochanter. The user can ensure any tissue is cleared from the femur registration marker 154. FIG. 85 is a view of the femur registration marker 154. The method can include placing the navigation device 102, assembled with registration probe 114, the reference sensor device 104, and the femur sensor device 106 on the pelvic bracket 110 back on the pelvic base 112 before initiating leg length and offset measurement. The method can include placing the thigh mount 122 back on the thigh plate 120 before proceeding if the thigh mount 122 has been removed. The method can include transferring the femur sensor device 106 from the pelvic bracket 110 to the thigh mount 122. FIG. 86 is a view of the femur sensor device 106 coupled with the thigh mount 122.

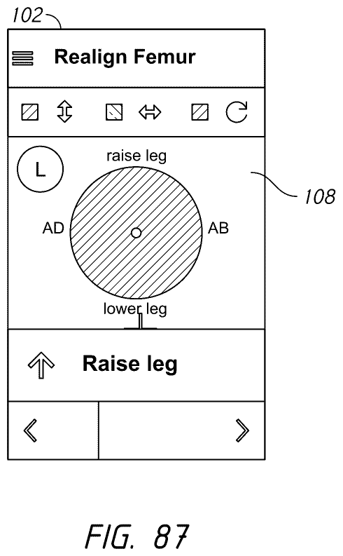

The method can include following instructions on the screen to reorient the leg into an acceptable orientation, if necessary. FIG. 87 is a view of a screen of the graphical user interface 108 of the navigation device 102. The screen can instruct the user to realign femur. The screen can provide instructions such as raise the leg of the patient. The screen can instruct the user to orient the femur near the same position as initial registration. The screen can provide a target visualization. The target visualization can be based on the origin being the same position as initial registration. The center of the target can indicate when the femur is positioned in the same position as initial registration. The inside of the target can indicate acceptable ranges relative to the initial registration. The target can provide a visual representation of the position of the femur relative to initial registration. The user can raise or lower the leg to move a marker relative to the target. The user can move the leg in abduction (AB) or adduction (AD) to move the marker relative to the target.

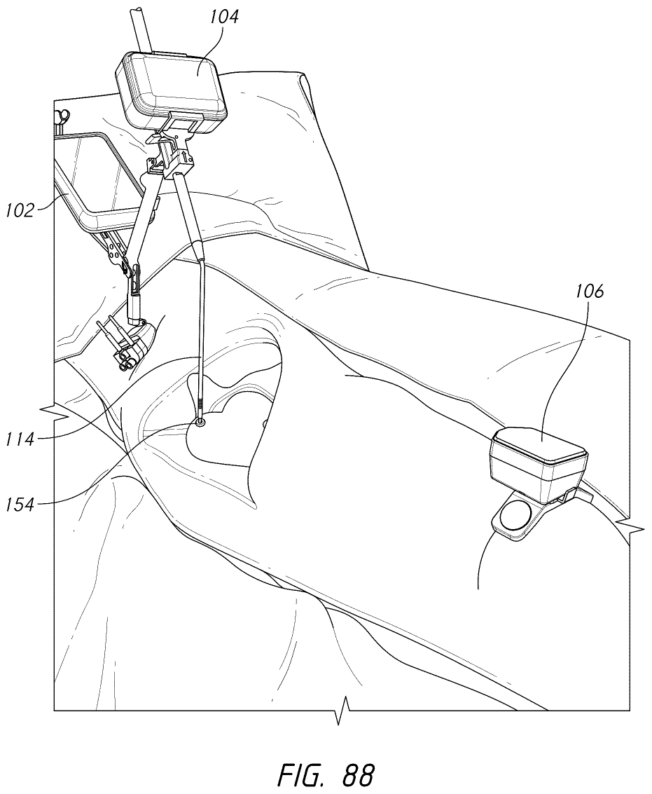

The method can include inserting the tip of the registration probe 114 into the head of the femur registration marker 154. The user can ensure that the registration probe 114 is fully seated into the head of the femur registration marker 154. The method can include holding the registration probe 114 steady. The method can include pressing register to register the femur registration marker 154. FIG. 88 is a view of the navigation device 102, the reference sensor device 104, the femur sensor device 106, and the registration probe 114.

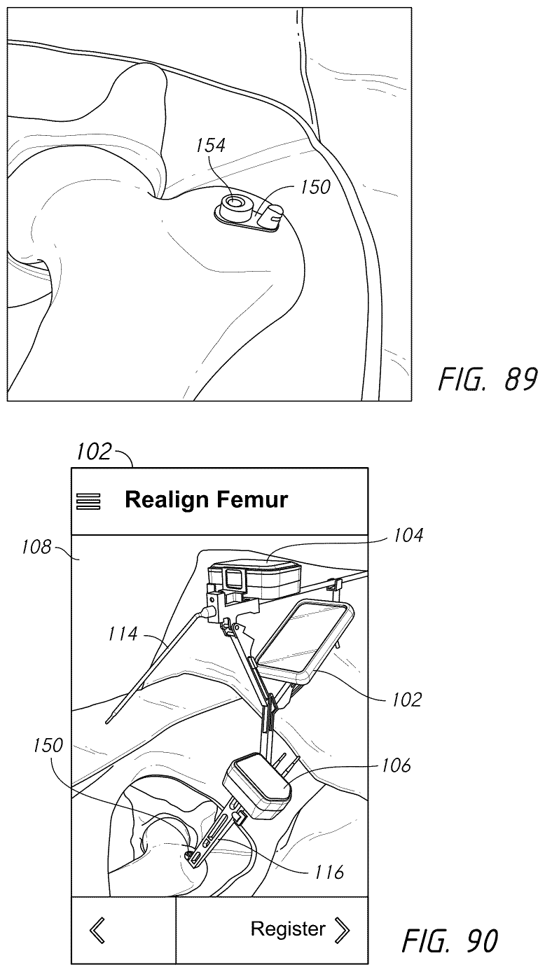

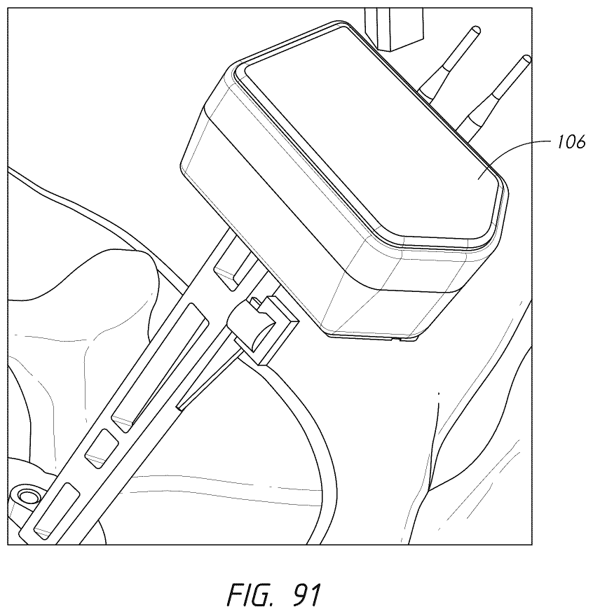

For the greater trochanter, the user can visually locate the fixation plate 150 and the femur registration marker 154 on the greater trochanter. The user can ensure any tissue is cleared from the femur registration marker 154. The user can hold the fixation tower 116. FIG. 89 is a view of the fixation plate 150. The method can include registering the femur. The method can include ensuring that the femur sensor device 106 is attached to the pelvic bracket 110 on the same side as during initial registration. The method can include transferring the femur sensor device 106 from the pelvic bracket 110 to the fixation tower 116. The method can include coupling the fixation tower 116 with the femur sensor device 106 to the fixation plate 150. FIG. 90 is a view of a screen of the graphical user interface 108 of the navigation device 102. The screen can instruct the user to register the femur. The method can include attaching the fixation tower with the attached femur sensor device 106 the fixation plate 150. The user can ensure that soft tissue does not press against the fixation tower 116. FIG. 91 is a view of the femur sensor device 106.

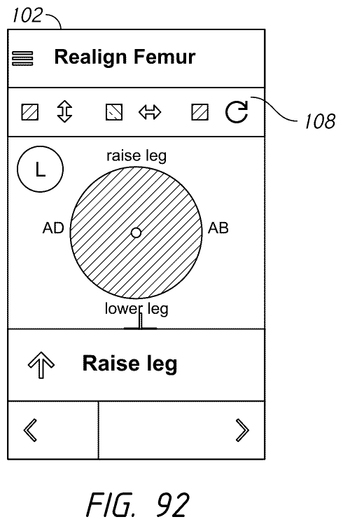

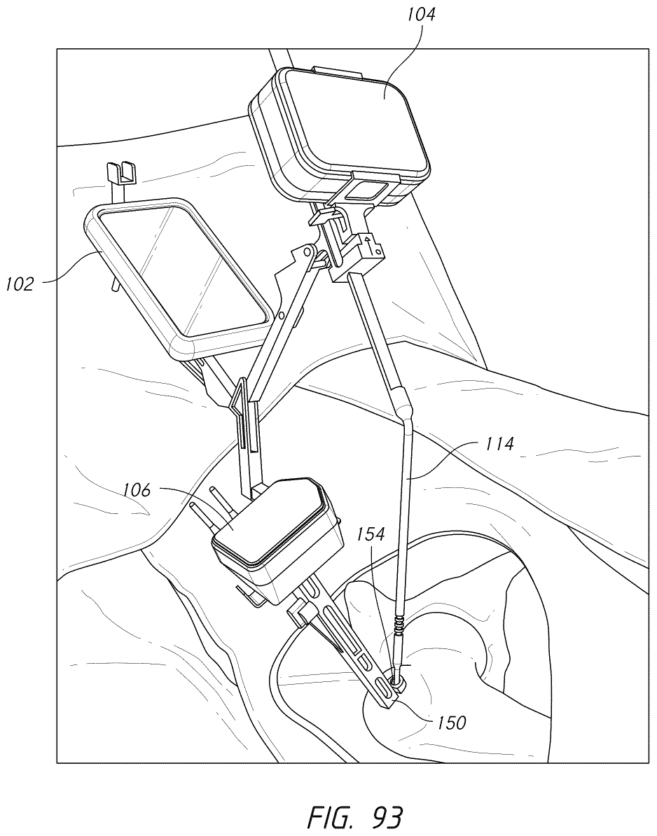

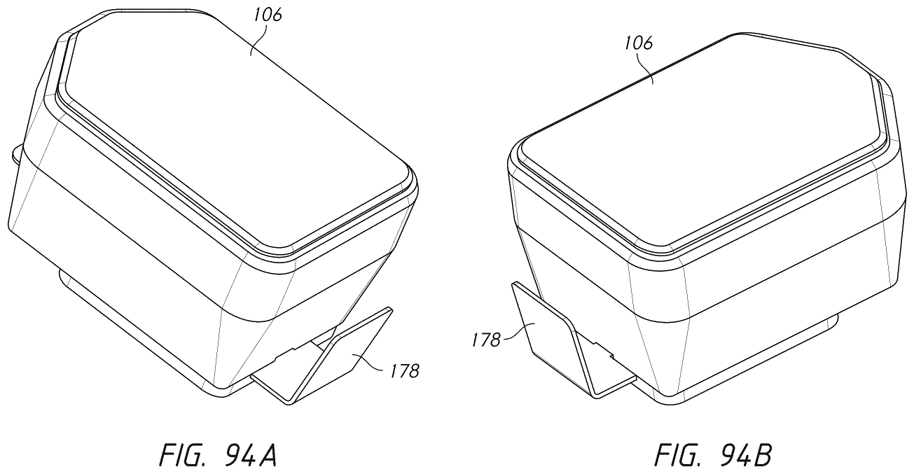

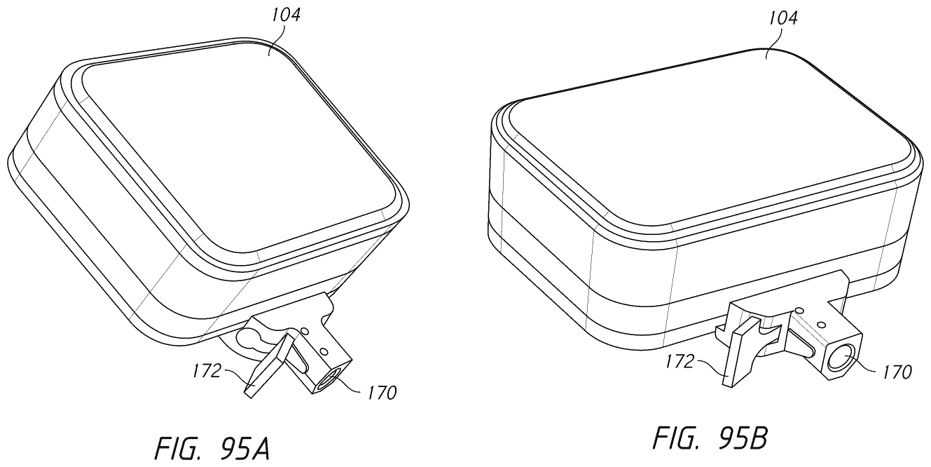

The method can include following instructions on the screen to reorient the leg into an acceptable orientation, if necessary. FIG. 92 is a view of a screen of the graphical user interface 108 of the navigation device 102. The screen can instruct the user to realign femur to be an acceptable orientation. The screen can provide instructions such as raise the leg or lower the leg. The screen can provide instructions such as move the leg in abduction (AB) or adduction (AD). The screen can provide a target visualization to assist in aligning the femur in the same position as initial registration.

The method can include inserting the tip of the registration probe 114 into the head of the femur registration marker 154. The user can ensure that the registration probe 114 is fully seated. The method can include holding the registration probe 114 steady. The method can include pressing register to register the femur registration marker 154. FIG. 93 is a view of the navigation device 102, the reference sensor device 104, the femur sensor device 106, and the registration probe 114.

The method can include measuring changes in leg length and offset by completing a registration of the femoral fixation. In some methods, the method can include using either the distal thigh or greater trochanter based on the user's earlier selection. The method can include viewing changes in leg length and joint offset on a screen of the graphical user interface 108 of the navigation device 102. In some methods, the leg length number can be displayed in green if the user selected a target change in leg length during the surgical planning step, and the registered change is within 3 mm of the target change. The method can include repeating the measurement for change in leg length and joint offset.

The method can include viewing a procedure summary. The method can include viewing data registered during the procedure on a screen of the graphical user interface 108 of the navigation device 102. The method can include viewing disposal instructions for the navigation device 102. The method can include viewing storage instructions for the reference sensor device 104 and/or the femur sensor device 106. The method can include viewing instructions for battery removal and disposal for the reference sensor device 104 and/or the femur sensor device 106. The method can include returning components of the hip navigation system 100 to the tray base 162. The method can include reusing the fixation pins 156, 158. The method can include reusing the reference sensor device 104. The method can include reusing the femur sensor device 106. The method can include powering off the navigation device 102. The method can include selecting the power off button. The method can include a hard power off. The method can include powering off the navigation device 102 by holding down power button for 3 seconds until the blue light illuminates. The method can include powering off the navigation device 102 with an additional input. The method can include powering off the navigation device 102 after 90 minutes without a button being pressed. The method can include selecting a button for ending the procedure.

Referring back to FIG. 1, the hip navigation system 100 is adapted to navigate to target angles. The hip navigation system 100 can be used for registering points, such as anatomical landmarks to determine a reference frame. The navigated angles are with respect to this reference frame. The hip navigation system 100 is shown mounted on a pelvis in FIG. 1. The method can include registering the femur prior to and after the joint is replaced to confirm an aspect of the relative position and/or orientation of the femur including leg length and joint offset.

The hip navigation system 100 can include the pelvic bracket 110, the pelvic base 112, and the fixation pins 156, 158. The pelvic bracket 110 can be rigidly connected to the pelvis so that motion of the pelvis causes corresponding motion of one or more inertial sensors of the navigation device 102. In some methods, sensing motion of the pelvis enables the hip navigation system 100 to eliminate movement of the patient as a source of error in the navigation. The registration probe 114 provides a full range of controlled motion relative to the pelvic bracket 110 and the one or more inertial sensors of the reference sensor device 104 are able to track the motion of the registration probe 114. The hip navigation system 100 can be used in combination with the impactor 190 coupled with the impactor adapter clip 144. The impactor 190 provides a free range of motion and the one or more inertial sensors of the femur sensor device 106 are able to track the motion of the impactor 190. The registration wand 132 provides a free range of motion and the one or more inertial sensors of the femur sensor device 106 are able to track the motion of the registration wand 132. The navigation device 102, the reference sensor device 104, and the femur sensor device 106 are configured to receive and/or transfer data among themselves wirelessly, using Bluetooth, wifi® or other standard wireless telemetry protocol.

In some embodiments, the hip navigation system 100 is configured to locate a reference frame to aid in proper placement of a prosthetic hip joint. In some methods, a reference frame can be located using the hip navigation system 100 that includes registering the two anterior superior iliac spines (ASIS). In some methods, a reference frame can be located using the hip navigation system 100 that includes registering the two anterior superior iliac spines (ASIS) and the pubic symphysis. The reference frame can be the anterior pelvic plane (APP). In some methods, a reference frame can be located using the hip navigation system 100 that includes registering the two anterior superior iliac spines (ASIS) and a measurement of gravity. The reference frame can be the adjusted plane. In some methods, a reference frame can be located using the hip navigation system 100 that includes registering the two anterior superior iliac spines (ASIS) and inputting a measurement based on the patient's anatomy. The reference frame can be the functional pelvic plane. The measurement based on the patient's anatomy can be a measurement of pelvic tilt from a standing lateral x-ray.

The hip navigation system 100 can configured to account for patient specific anatomical variability. In some methods, a correspondence between one or more patient specific measurements can be utilized as input for the reference frame for the navigated angles. The user can use standard radiographic imaging to provide an anterior pelvic bone image. This image can be read to derive the pelvic tilt angle pre-operatively. The navigation device 102 can allow the user can enter a numerical value for the functional pelvic plane angle. The pre-operative image can be used to enhance alignment of a cup in a hip procedure by providing patient specific data. The pelvic tilt angle is patient specific. The angle can be entered with the graphical user interface 108 of the navigation unit 102 to provide a patient specific reference frame. The angle can be a measurement of pelvic tilt.

The pelvic bracket 110 can be configured to couple to the navigation device 102, the reference sensor device 104, and the femur sensor device 106 during calibration. The pelvic bracket 110 can be configured to couple to the navigation device 102, the reference sensor device 104, and the femur sensor device 106 during the surgical procedure. The pelvic bracket 110 can be configured to couple to a bone of the patient, such as the pelvis with the use of the pelvic base 112 and the fixation pins 156, 158. The pelvic bracket 110 can include a first interface to couple with the navigation device 102. The navigation device 102 is configured to couple to the first interface, wherein the navigation device 102 comprises one or more sensors configured to sense changes in orientation. The first interface can be the magnetic section 165 of the pelvic bracket 110. The navigation device 102 can have a corresponding magnetic section configured to securely couple to the magnetic section 165 of the pelvic bracket 110. The navigation device 102 can couple to the first interface to track motion of the pelvis. The navigation device 102 can couple to the first interface to enable the screen of the graphical user interface 108 to be viewable by the user. The navigation device 102 can couple to the first interface to enable transfer of data from the reference sensor device 104 and the femur sensor device 106. In some embodiments, the first interface is different than the second interface. In some embodiments, the first interface is different than the third interface. In some embodiments, the first interface only couples to the navigation device 102.

The pelvic bracket 110 can include a second interface to couple with the reference sensor device 104. The reference sensor device 104 is configured to couple to the second interface, wherein the reference sensor device 104 comprises one or more sensors configured to sense changes in orientation. The second interface can be the coupler 168 of the pelvic bracket 110. The reference sensor device 104 can have a corresponding receiver 170 configured to securely couple to the coupler 168 of the pelvic bracket 110. The user can depress the lever 172 on the reference sensor device 104 to engage the coupler 168 of the pelvic bracket 110. In some methods, the reference sensor device 104 can couple to the coupler 168 of the pelvic bracket 110 during calibration.

The registration probe 114 is configured to couple to the second interface. The registration probe 114 can include a corresponding receiver 182. The corresponding receiver 182 of the registration probe 114 is configured to securely couple to the coupler 168 of the pelvic bracket 110. The registration probe 114 can include the coupler 180. The reference sensor device 104 can have the corresponding receiver 170 configured to securely couple to the coupler 180 of the registration probe 114. The user can depress the lever 172 on the reference sensor device 104 to engage the coupler 180 of the registration probe 114. The user can depress the lever 184 on the registration probe 114 to engage the coupler 168 of the pelvic bracket 110. In some methods, the registration probe 114 can couple to the coupler 168 of the pelvic bracket 110 during the surgical procedure. The reference sensor device 104 can couple to the registration probe 114 and therefore to the pelvic bracket 110 to enable registering points. The reference sensor device 104 can couple to the registration probe 114 and therefore to the pelvic bracket 110 to enable registering points of a reference frame, such as anatomical landmarks. The reference sensor device 104 can couple to the registration probe 114 and therefore to the pelvic bracket 110 to enable registering a point before and after cup replacement to determine leg length and joint offset. The reference sensor device 104 can couple to the registration probe 114 and therefore to the pelvic bracket 110 to enable registering a point to determine center of rotation.

In some embodiments, the coupler 168 of the pelvic bracket 110 and the coupler 180 of the registration probe 114 are identical. In some embodiments, the coupler 168, 180 can include an elongated post. In some embodiments, the coupler 168, 180 can have an irregular shape (e.g., triangular, teardrop, elliptical, rectangular). The irregular shape can facilitate alignment of the registration probe 114 and/or the reference sensor device 104 with the corresponding couplers 168, 180. In some embodiments, the coupler 168, 180 can allow the registration probe 114 and/or the reference sensor device 104 to mate with the coupler 168, 180 in a single orientation. In some embodiments, the second interface is different than the third interface. In some embodiments, the second interface only couples to the registration probe 114 or the reference sensor device 104.

The pelvic bracket 110 can include a third interface to couple with the femur sensor device 106. The femur sensor device 106 is configured to couple to the third interface, wherein the femur sensor device 106 comprises one or more sensors configured to sense changes in orientation. The third interface can be the coupler 174 of the pelvic bracket 110. The femur sensor device 106 can have a corresponding receiver 176 configured to securely couple to the coupler 174 of the pelvic bracket 110. The user can depress the lever 178 on the femur sensor device 106 to engage the coupler 174 of the pelvic bracket 110. The femur sensor device 106 can be coupled to the coupler 174 on the left side of the pelvic bracket 110. The femur sensor device 106 can be positioned on the left side of the pelvic bracket 110 for easier access to the lever 172 of the reference sensor 104. In some embodiments, the pelvic bracket 110 can have a pair of couplers 174, with one coupler 174 disposed on the right side of the pelvic bracket 110 and one coupler 174 disposed on the left side of the pelvic bracket 110.

The femur sensor device 106 can couple to the third interface to eliminate errors during navigation. The femur sensor device 106 can couple to the third interface to before and after coupling to the impactor 190. The femur sensor device 106 can couple to the third interface to before and after coupling to the registration wand 132. In some embodiments, the third interface only couples to the femur sensor device 106.

The femur sensor device 106 can be configured to be transferred between locations during the surgical procedure. The femur sensor device 106 can be coupled to the pelvic bracket 110. The femur sensor device 106 can be transferred from the pelvic bracket 110. In some methods, the femur sensor device 106 can be transferred to the fixation tower 116 for registration on the greater trochanter. The femur sensor device 106 can be transferred from the fixation tower 116. The femur sensor device 106 can be transferred back to the pelvic bracket 110. In some methods, the femur sensor device 106 can be transferred to the thigh mount 112 for registration on the distal thigh. The femur sensor device 106 can be transferred from the thigh mount 112. The femur sensor device 106 can be transferred back to the pelvic bracket 110. The femur sensor device 106 can be transferred to impactor adapter clip 144. The femur sensor device 106 can be transferred from the impactor adapter clip 144. The femur sensor device 106 can be transferred to registration wand 132. The femur sensor device 106 can be transferred from the registration wand 132. The hip navigation system 100 can include multiple locations that receive the femur sensor device 106.

The navigation device 102, the reference sensor device 104, and/or the femur sensor device 106 are configured to communicate changes in orientation therebetween. In some methods, the reference sensor device 104 and/or the femur sensor device 106 communicate changes in orientation to the navigation device 102. In some methods, the reference sensor device 104 and/or the femur sensor device 106 communicate changes in rotation to the navigation device 102. In some methods, the reference sensor device 104 communicates changes in position to the navigation device 102. The navigation device 102, the reference sensor device 104, and/or the femur sensor device 106 are configured to register a reference frame. The navigation device 102, the reference sensor device 104, and/or the femur sensor device 106 are configured to measure one or more angles of the impactor 190 during cup placement. The navigation device 102, the reference sensor device 104, and/or the femur sensor device 106 are configured to determine a center of rotation. The navigation device 102, the reference sensor device 104, and/or the femur sensor device 106 are configured to register a point before and after cup replacement to determine leg length and/or joint offset. The navigation device 102, the reference sensor device 104, and/or the femur sensor device 106 are configured to perform a virtual transformation about the center of rotation. The navigation device 102, the reference sensor device 104, and/or the femur sensor device 106 are configured to perform virtually reorient the femur relative to the pelvis for leg length and/or joint offset measurements. The pelvic bracket 110 can couple to navigation device 102, the reference sensor device 104, and the femur sensor device 106 during the surgical procedure. The femur sensor 106 can be transferred to other instrumentation during the surgical procedure. The hip navigation system can include the use of three inertial sensor devices.

The hip navigation system 100 can be adapted for various techniques. The hip navigation system 100 can include any component described herein. The hip navigation system 100 can be used in any technique or method step described herein. The hip navigation system 100 can be adapted for registering the femur prior to and after the cup is replaced to confirm leg length and/or joint offset. In some methods, the hip navigation system 100 can register a leg length direction which is utilized for measurements of leg length and/or joint offset. In some methods, the hip navigation system 100 can register a center of rotation which is utilized for measurements of leg length and/or joint offset. In some methods, the hip navigation system 100 can register a point before and after cup replacement which is utilized for measurements of leg length and/or joint offset.

The method can include determining a leg length direction. The pelvic bracket 110 can couple to navigation device 102, the registration probe 114 coupled with the reference sensor device 104, and the femur sensor device 106. The pelvic bracket 110 can couple to the pelvic base 112. The registration probe 114 can be moved into alignment with the long axis of the femur. The navigation device 102 can register the leg length direction when a corresponding button is pressed on the screen of the graphical user interface 108 of the navigation device 102. The navigation device 102 can register the leg length direction by recording the orientation of the reference sensor device 104 when the registration probe 114 is aligned with the long axis of the femur. In some methods, the leg length direction is utilized in the measurement of leg length and/or joint offset. In some methods, the leg length direction is utilized determining the direction for leg length and/or joint offset. In some methods, the registration of leg length direction can define the axis of the femur.

The method can include determining a center of rotation. The hip navigation system 100 can include the registration wand 132. The hip navigation system 100 can include one or more liner registration trials 134, 136, 138, 140, 142. The hip navigation system 100 can include a plurality of liner registration trials 134, 136, 138, 140, 142 having different diameters. The registration wand 132 can couple to one liner registration trial 134 of the plurality of liner registration trials 134, 136, 138, 140, 142 that corresponds to the diameter of the liner 196 of the cup 194. The method can include selecting the appropriately sized liner registration trial 134 that mates with the implanted liner 196. The liner registration trial 134 can be coupled to the registration wand 132, such as through threads. The liner registration trial 134 is fully inserted into the implanted liner 196. The spherical shape of the liner registration trial 134 should fully seat within the implanted liner 196.

The navigation device 102 can be coupled to the pelvic bracket 110. The registration probe 114 with the reference sensor device 104 can be coupled to the pelvic bracket 110. The femur sensor device 106 can be coupled to the pelvic bracket 110. The femur sensor device 106 can be transferred from the pelvic bracket 110 to the registration wand 132. The registration wand 132 can include the coupler 179. The femur sensor device 106 can have a corresponding receiver 176 configured to securely couple to the coupler 179 of the registration wand 132. The user can depress a lever 178 on the femur sensor device 106 to engage the coupler 179 of the registration wand 132. The user can ensure the foot of the lever 178 of the femur sensor device 106 is pointed away from the navigation unit 102.

The method can include determining a center of rotation. The center of rotation can correspond to the center of rotation of the registration wand 132. The center of rotation can correspond to the center of rotation of the implanted liner 196. The center of rotation can correspond to the center of rotation of the implanted cup 194. The center of rotation can correspond to the center of rotation of the head of the femur.

The pelvic bracket 110 can couple to the registration probe 114 with the reference sensor device 104. The tip of the registration probe 114 can be moved into the divot 177 of the registration wand 132. The navigation device 102 can register the orientation of the reference sensor device 104 and the orientation of the femur sensor 106 when a corresponding button is pressed on the screen of the graphical user interface 108 of the navigation device 102. The orientation of the reference sensor device 104 when the tip of the registration probe 114 contacts the divot 177 is mathematically related to the center of rotation. The orientation of the femur sensor device 106 when the liner registration trial 134 is fully inserted into the implanted liner 196 is mathematically related to the center of rotation. The navigation device 102 can mathematically derive the center of rotation from the orientation, rotation, and/or position of the reference sensor device 104 and the orientation and/or rotation of the femur sensor device 106. The method can include pressing a register button on the graphical user interface 108 of the navigation device 102 to register the registration wand 132 when the tip of the registration probe 114 contacts the divot 177 and when the liner registration trial 134 is fully inserted into the implanted liner 196. The method of determining the center of rotation can be repeated if the implanted liner 196 is replaced.

The method can include registering a point before and after cup replacement. The hip navigation system 100 can include components for mounting the femur sensor device 106 on the distal thigh. The components can include the thigh plate 120 and the thigh mount 122 for mounting the femur sensor device 106 on the distal thigh. The hip navigation system 100 can include components for mounting the femur sensor device 106 on the greater trochanter. The components can include the fixation plate 150 and the fixation tower 116 for mounting the femur sensor device 106 on the greater trochanter. The hip navigation system 100 can include the femur registration marker 154. The femur registration marker 154 can be a tack or screw. The femur registration marker 154 can be mounted on the greater trochanter. The femur registration marker 154 can be mounted within the fixation plate 150. The tip of the registration probe 114 can contact and register the femur registration marker 154 before the cup is placed. The reference sensor device 104 can transmit data to the navigation device 102 when a button is pressed to register the femur registration marker 154 before the cup is replaced. The reference sensor device 104 can provide data related to the position, orientation, and/or rotation of the femur registration marker 154. The femur sensor device 106 can transmit data to the navigation device 102 when a button is pressed to register the femur registration marker 154 before the cup is placed. The femur sensor device 106 can provide data related to the orientation and/or rotation of the femur. The data from the reference sensor device 104 and the femur sensor device 106 can be simultaneously transmitted to the navigation device 102 when a button is pressed to register the femur registration marker 154 before the cup is placed.

The femur registration marker 154 remains mounted on the greater trochanter during cup placement. The femur registration marker 154 can provide a point which is fixed relative to the femur. The femur registration marker 154 is easy to find for repeat registrations. The femur registration marker 154 is a low-profile component. In some methods, the fixation plate 150 remains mounted to the greater trochanter during cup placement. In some methods, the thigh plate 120 remains mounted to the distal thigh during cup placement. In some methods, the thigh mount 122 remains mounted to the distal thigh during cup placement. The tip of the registration probe 114 with the reference sensor device 104 can contact the femur registration marker 154 located on the greater trochanter after the cup is placed. The reference sensor device 104 can transmit data to the navigation device 102 when a button is pressed to register the femur registration marker 154 after the cup is replaced. The reference sensor device 104 can transmit data to the navigation device 102 related to orientation, rotation, and/or position. The reference sensor device 104 can transmit data to the navigation device 102 related to distance. The reference sensor device 104 can include the camera 181 which can capture images of the marking 179. The reference sensor device 104 and/or the navigation device can calculate a distance measurement from the captured image. The femur sensor device 106 can transmit data to the navigation device 102 when a button is pressed to register the femur registration marker 154 after the cup is replaced. The femur sensor device 106 can transmit data to the navigation device 102 related to orientation and/or rotation. The femur sensor device 106 can transmit data to the navigation device 102 related to the position of the femur. The femur sensor device 106 can transmit data to the navigation device 102 related to the rotational orientation of the femur relative to the pelvis.

The navigation device 102 can correct for changes in the femur angle with respect to the pelvis. The navigation device 102 can include software on a processor that utilizes the center of rotation calculated during the procedure. The navigation device 102 can correct for changes in the femur between the baseline measurement before the cup 194 was placed and the later measurement after the cup 194 was placed. The navigation device 102 can register the femur registration marker 154 before the cup is placed and register the femur registration marker 154 after the cup is placed. The navigation device 102 can correct for changes in orientation by doing a virtual rotation of the femur about the center of rotation. The navigation device 102 can register one point on the femur before the cup is placed. The navigation device 102 can register the center of rotation. The navigation device 102 can register one point on the femur after the cup is placed. The navigation device 102 can register the rotational orientation of the femur sensor device 106 before the cup 194 is placed. The navigation device 102 can register the rotational orientation of the femur sensor device 106 after the cup 194 is placed. The navigation device 102 can resolve for changes in the femur orientation after the cup is placed.

The navigation device 102 can allow the user to place the femur relative to the pelvis within a range of positions. The graphical user interface 108 of the navigation device 102 can guide the user in replicating the orientation of the femur relative to the pelvis after the cup is placed. The femur can be repositioned within +/−15° flexion from the baseline position. The femur can be repositioned within +/−15° abduction from the baseline position. The femur can be repositioned within +/−15° adduction from the baseline position. The target on a screen of the graphical user interface 108 of the navigation device 102 can provide instructions to the user to reposition the femur within the range of positions.

For the distal thigh, the thigh plate 120 remains mounted on the distal thigh during cup placement. The thigh plate 120 can provide a point which is fixed relative to the femur. The thigh plate 120 can provide a point which is fixed for mounting the femur sensor device 106 before and after cup placement. The thigh plate 120 is easy to find for repeat registrations. The thigh plate 120 is a low-profile component. The femur sensor device 106 can be returned to the same fixed point by mounting to the thigh mount 122 coupled to the thigh plate 120. For the greater trochanter, the fixation plate 150 remains mounted on the greater trochanter during cup placement. The fixation plate 150 can provide a point which is fixed relative to the femur. The fixation plate 150 is easy to find for repeat registrations. The fixation plate 150 is a low-profile component. The femur sensor device 106 can be returned to the same fixed point by mounting to the fixation tower 116 coupled to the fixation plate 150.

The femur sensor device 106 can track the femur in space. The femur sensor device 106 can track the femur in real-time. The femur sensor device 106 can determine the angle of the femur relative to the pelvis. The navigation device 102 can utilize the angle of the femur from the femur sensor device 106 to perform calculations to virtually rotate the femur about the center of rotation back to the baseline position. The navigation device 102 can register the orientation and/or rotation of the femur sensor device 106 when femur registration marker 154 is registered before the cup is placed. The navigation device 102 can register the orientation and/or rotation of the femur sensor device 106 when the femur registration marker 154 is registered after the cup is placed. The navigation device 102 can determine the angle between the baseline measurement of the femur sensor device 106 and the later measurement of the femur sensor device 106. The angle can represent the angle of rotation of the femur between the baseline measurement and the later measurement. Instead of the user perfectly repositioning the femur relative to the pelvis in the same position after cup placement, the calculated angle can be used to virtually rotate the femur relative to the pelvis. This virtual repositioning facilitates accurate measurements of leg length and/or joint offset by accounting for the variation in rotational position of the femur between the baseline measurement and the later measurement.

In some methods, the navigation device 102 remains coupled with the pelvic bracket 110 during the cup placement. In some methods, the registration probe 114 remains coupled with the pelvic bracket 110 during the cup placement. In some methods, the reference sensor device 104 remains coupled with the registration probe 114 during the cup placement.

In some methods, the femur sensor device 106 is transferred from the pelvic bracket 110 for baseline measurements for leg length and joint offset. In some methods, the femur sensor device 106 is transferred from the pelvic bracket 110 to the femoral fixation on the distal thigh. In some methods, the femur sensor device 106 is transferred from the pelvic bracket 110 to the femoral fixation on the greater trochanter. In some methods, the femur sensor device 106 is transferred from the femoral fixation back to the pelvic bracket 110.

In some methods, the femur sensor device 106 is transferred from the pelvic bracket 110 for cup navigation. In some methods, the femur sensor device 106 is transferred from the pelvic bracket 110 to the impactor 190. In some methods, the femur sensor device 106 is transferred from the pelvic bracket 110 to the impactor adapter clip 144. The impactor adapter clip 144 can be coupled to the impactor 190. In some methods, the impactor adapter clip 144 only couples with the femur sensor device 106. In some methods, the impactor adapter clip 144 does not couple with the reference sensor device 104. In some methods, the femur sensor device 106 is transferred from the impactor adapter clip 144 back to the pelvic bracket 110.

In some methods, the femur sensor device 106 is transferred from the pelvic bracket 110 for later measurements for leg length and/or joint offset. In some methods, the femur sensor device 106 is transferred from the pelvic bracket 110 back to the femoral fixation on the distal thigh. In some methods, the femur sensor device 106 is transferred from the pelvic bracket 110 back to the femoral fixation on the greater trochanter. In some methods, the femur sensor device 106 is transferred from the femoral fixation back to the pelvic bracket 110.

The method can include orienting the cup 194 in the acetabulum using the impactor 190 and the femur sensor device 106, wherein during orienting, inertial data from the femur sensor device 106 is used to confirm a proper orientation of the cup 194. The cup 194 can be inserted into the acetabulum and positioned at the target angle. The graphical user interface 108 of the navigation device 102 can provide a target to visualize the correct orientation. The surgical navigation device 102 can display when the femur sensor device 106 is located at the target abduction and anteversion angles entered previously. The navigation device 102 can display the abduction and anteversion angles of the impactor 190 as the impactor 190 is moved. The user can press a button on the graphical user interface 108 of the navigation device 102 to record the abduction and anteversion angles of the cup 194. The navigation device 102 can output abduction and anteversion angles according to radiographic definitions. Anteversion (Radiographic Anteversion) is the angle between the acetabular axis and the coronal plane. Abduction (Radiographic Abduction/Inclination) is the coronal plane projection of the angle between the acetabular axis and the longitudinal axis of the body. The navigation device 102 can output abduction and anteversion angles relative to the anterior pelvic plane (APP). The navigation device 102 can output abduction and anteversion angles relative to the functional pelvic plane (FPP). The navigation device 102 can output abduction and anteversion angles relative to the coronal plane. Once the femur sensor device 106 is coupled to the impactor 190, the navigation device 102 can display the abduction and anteversion angles of the impactor 190 relative to the selected reference frame. In some methods, the user can change the reference frame to view navigated angles relative to an alternative reference frame.

FIGS. 94A-94B are views of the femur sensor device 106. The femur sensor device 106 can have an elongated shape. The femur sensor device 106 can have a head and a foot. The head of the femur sensor device 106 can be oriented toward the head of the patient. The user can maintain the head of the femur sensor device 106 pointing toward the head of the patient as the femur sensor device 106 is transferred between components of the hip navigation system 100. In some embodiments, the head of the femur sensor device 106 is marked with text and/or an icon, such as an arrow. In some embodiments, the femur sensor device 106 can be oriented in use such that the correct end points toward the head of the patient. The femur sensor device 106 can include the lever 178. The lever 178 can be located at the foot of the femur sensor device 106. The lever 178 can be oriented toward the foot of the patient. The user can maintain the foot of the femur sensor device 106 pointing toward the foot of the patient as the femur sensor device 106 is transferred between components of the hip navigation system 100. In some embodiments, the foot of the femur sensor device 106 is marked with text and/or an icon, such as an arrow. In some embodiments, the femur sensor device 106 can be oriented in use such that the lever 178 points toward the foot of the patient. The femur sensor device can include the femur sensor identification number.

The user can depress the lever 178 on the femur sensor device 106 to engage the coupler 174 of the pelvic bracket 110. The femur sensor device 106 can be mounted on the pelvic bracket 110 for calibration. The femur sensor device 106 can be mounted on the pelvic bracket 110 when the registration probe 114 is contacting one or more points on the probe calibration jig 128. The femur sensor device 106 can be mounted on the pelvic bracket 110 when the registration probe 114 is contacting one or more points to define a reference frame. The user can depress the lever 178 on the femur sensor device 106 to engage the coupler 189 of the impactor adapter clip 144. The femur sensor device 106 can be mounted on the impactor adapter clip 144 when the impactor adapter clip 144 is mounted to the impactor 190. The femur sensor device 106 can be mounted on the impactor adapter clip 144 when the cup is being navigated toward target angles. The femur sensor device 106 can be removed from the impactor 190 during impaction of the cup 194. The user can depress the lever 178 on the femur sensor device 106 to engage the coupler 123 of the thigh mount 122. The femur sensor device 106 can be mounted on the thigh mount 122 when the registration probe 114 and the reference sensor device 106 register a point for leg length and/or joint offset before the cup is placed. The femur sensor device 106 can be mounted on the thigh mount 122 when the registration probe 114 and the reference sensor device 106 register a point for leg length and/or joint offset after the cup is placed. The user can depress the lever 178 on the femur sensor device 106 to engage the coupler 179 of the registration wand 132. The femur sensor device 106 can be mounted on the registration wand 132 for determining a center of rotation of the registration wand 132. The femur sensor device 106 can be mounted on the registration wand 132 when the registration probe 114 and the reference sensor device 106 register a point for the center of rotation. The femur sensor device 106 can transfer between a location on the pelvic bracket 110 and a location on the impactor adapter clip 144. The femur sensor device 106 can transfer between a location on the pelvic bracket 110 and a location on the thigh mount 122. The femur sensor device 106 can transfer between a location on the pelvic bracket 110 and a location on the registration wand 132.

FIGS. 95A-95B are views of the reference sensor device 104. The reference sensor device 104 can have a larger shape than the femur sensor device 106. The reference sensor device 104 can have the receiver 170 configured to couple to the coupler 168 of the pelvic bracket 110. The user can depress the lever 172 on the reference sensor device 104 to engage the coupler 168 of the pelvic bracket 110. The reference sensor device 104 can have the receiver 170 configured to couple to the coupler 180 of the registration probe 114. The user can depress the lever 172 on the reference sensor device 104 to engage the coupler 180 of the registration probe 114. The reference sensor device 104 can transfer between a location on the pelvic bracket 110 and a location on the registration probe 114. The reference sensor device 104 can be mounted on the pelvic bracket 110 for calibration. The reference sensor device 104 can be mounted on the registration probe 114 for calibration with the probe calibration jig 128. The reference sensor device 104 can be mounted on the registration probe 114 for registering one or more points for defining a reference frame. The reference sensor device 104 can be mounted on the registration probe 114 for registering a point for leg length and/or joint offset. The reference sensor device 104 can be mounted on the registration probe 114 for registering a point on the registration wand 132.

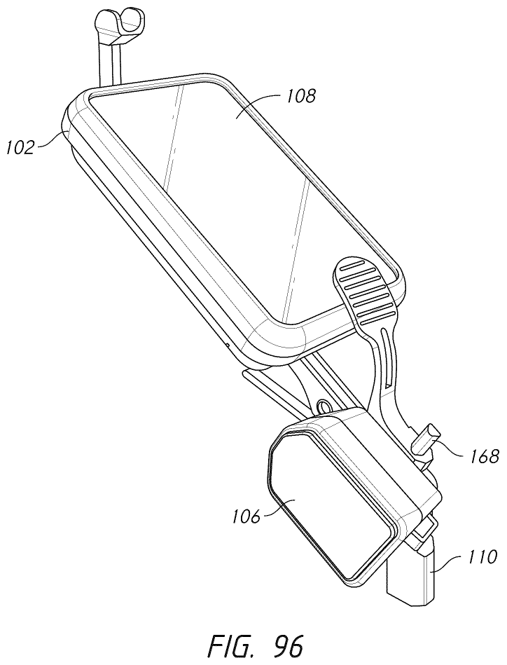

FIG. 96 is a view of the navigation device 102 and the femur sensor device 106. The navigation device 102 and the femur sensor device 106 can be mounted to the pelvic bracket 110. The femur sensor device 106 can have a low profile to avoid obstructing the graphical user interface 108 of the navigation device 102. The navigation device 102 can be angled relative to the coupler 168 of the pelvic bracket 110. The registration probe 114 can be mounted to the coupler 168 of the pelvic bracket 110. The reference senor device 104 can have a low profile to avoid obstructing the graphical user interface 108 of the navigation device 102. In some embodiments, the pelvic bracket 110 couples to the navigation device 102 in a single orientation. In some embodiments, the pelvic bracket 110 couples to the navigation device 102 such that the navigation device is oriented to be viewed by the user. In some embodiments, the pelvic bracket 110 couples to the navigation device 102 such that the on-screen graphics are not upside down. In some embodiments, the pelvic bracket 110 couples to the femur sensor device 106 in a single orientation. In some embodiments, the pelvic bracket 110 couples to the femur sensor device 106 such that the head of the femur sensor device 106 points toward the head of the patient and the foot of the femur sensor device 106 points toward the foot of the patient. In some embodiments, the pelvic bracket 110 couples to the registration probe 114 in a single orientation. In some embodiments, the registration probe 114 couples to the reference sensor device 104 in a single orientation. In some embodiments, the pelvic bracket 110 couples to the reference sensor device 204 in a single orientation.

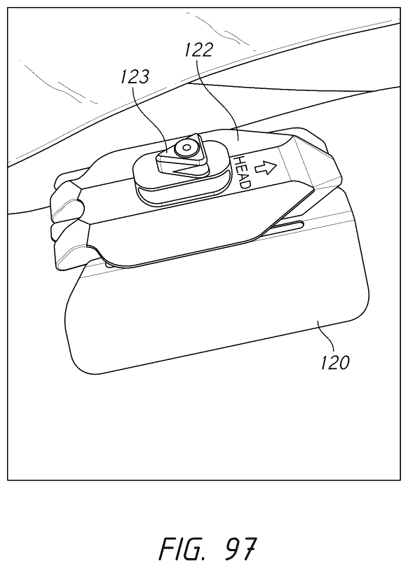

FIG. 97 is a view of the thigh plate 120 and the thigh mount 122. The thigh plate 120 can be configured to be affixed to skin on the distal thigh of the patient. The user can select a location on the distal thigh with little skin movement. The user can select a location on the distal thigh approximately 4-5 cm superior to the superior pole of the patella. The user can wrap the distal thigh in Coban™, or similar self-adherent wrap with latex. The user can wrap the distal thigh to minimize skin movement. The user can place the thigh plate 120 onto the Coban™. The user can secure the thigh plate 120 to the Coban™ using a small strip of Ioban™, or similar antimicrobial incise drape. The user can place one or more horizontal strips and/or one or more vertical strip of Ioban™ on the thigh plate 120. The thigh plate 120 can have a low-profile configuration.

The thigh mount 122 can couple to the thigh plate 120. In some embodiments, the thigh mount 122 and the thigh plate 120 are magnetic. In some embodiments, the thigh mount 122 and the thigh plate 120 comprise a mechanical connection such as snap fit. In some embodiments, the thigh mount 122 and the thigh plate 120 are coupled via adhesive or one or more horizontal strips and/or one or more vertical strip of Ioban™M. In some embodiments, the thigh mount 122 and the thigh plate 120 are integrally formed. In some embodiments, the thigh mount 122 couples to the thigh plate 120 in a single orientation.

The thigh mount 122 can include can coupler 123. The femur sensor device 106 can have the receiver 176 configured to securely couple to the coupler 123 of the thigh mount 122. The user can depress the lever 178 on the femur sensor device 106 to engage the coupler 123 of the thigh mount 122.

The thigh mount 122 can have an elongated shape. In some embodiments, the thigh mount 122 can be oriented in use such that the correct end points toward the head of the patient. The thigh mount 122 can have a head and a foot. The head of the thigh mount 122 can be oriented toward the head of the patient. The user can maintain the head of the thigh mount 122 pointing toward the head of the patient. In some embodiments, the head of the thigh mount 122 is marked with text and/or an icon, such as an arrow. In some embodiments, the thigh mount 122 couples to the femur sensor device 106 such that the head of the femur sensor device 106 points toward the head of the patient. In some embodiments, the femur sensor device 106 couples to the thigh mount 122 in a single orientation. In some embodiments, the femur sensor device 106 can be oriented in use such that the correct end points toward the head of the patient.

In some embodiments, the method of use of the hip navigation system 100 can include any steps described herein. In some embodiments, the method of use of the hip navigation system 100 can omit any steps described herein.

The method can include installing the reference sensor battery 164 in the reference sensor device 104 and installing the femur sensor battery assembly 166 in the femur sensor device 106. The method can include electronic pairing of the navigation device 102, the reference sensor device 104, and the femur sensor device 106. The method can include calibration. The user can move the pelvic bracket 110 coupled with the navigation device 102, the reference sensor device 104, and the femur sensor device 106 through a sequence of positions. The method can include calibration of the registration probe 114. The user can move the registration probe 114 to contact points of the probe calibration jig 128. The method can include coupling the fixation pins 156, 158, the pelvic base 112, and the pelvic bracket 110 to the pelvis of the patient. The method can include coupling the navigation device 102, the femur sensor device 106, and the registration probe 114 to the pelvic bracket 110. The method can include coupling the reference sensor device 104 to the registration probe 114.

In some embodiments, the methods of use of the hip navigation system 100 can include interacting with the graphical user interface 108 of the navigation device 102 for selecting options for the surgical procedure. In some embodiments, the graphical user interface 108 of the navigation device 102 does not provide the user with the option for locations of femoral fixation. The surgical procedure can be limited to the distal thigh location and the user does not need to select a location.

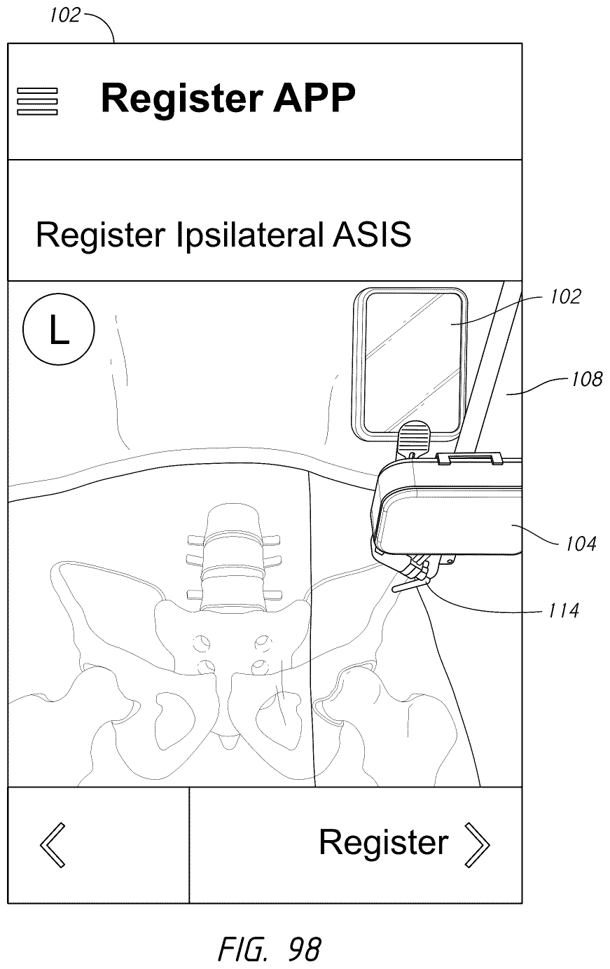

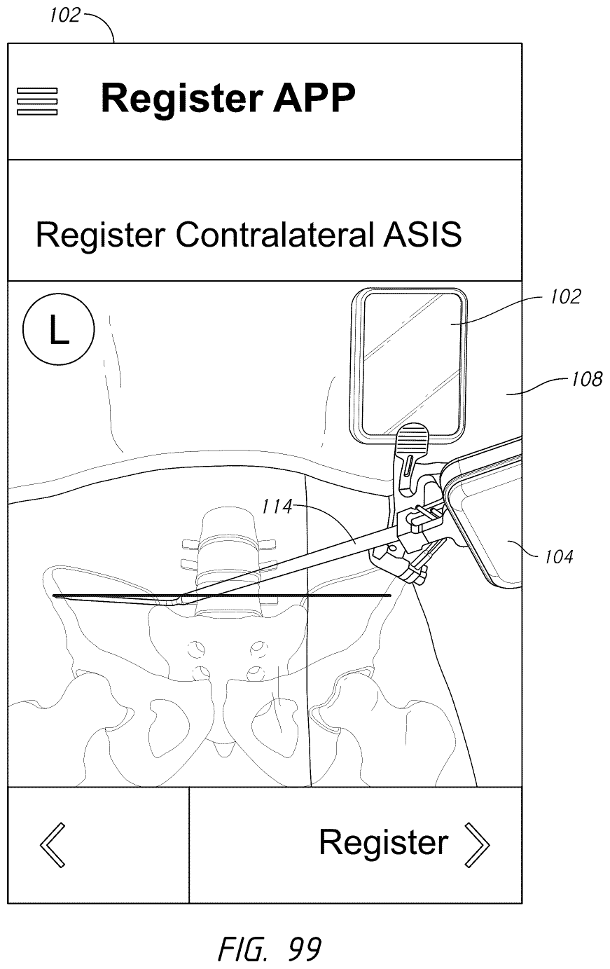

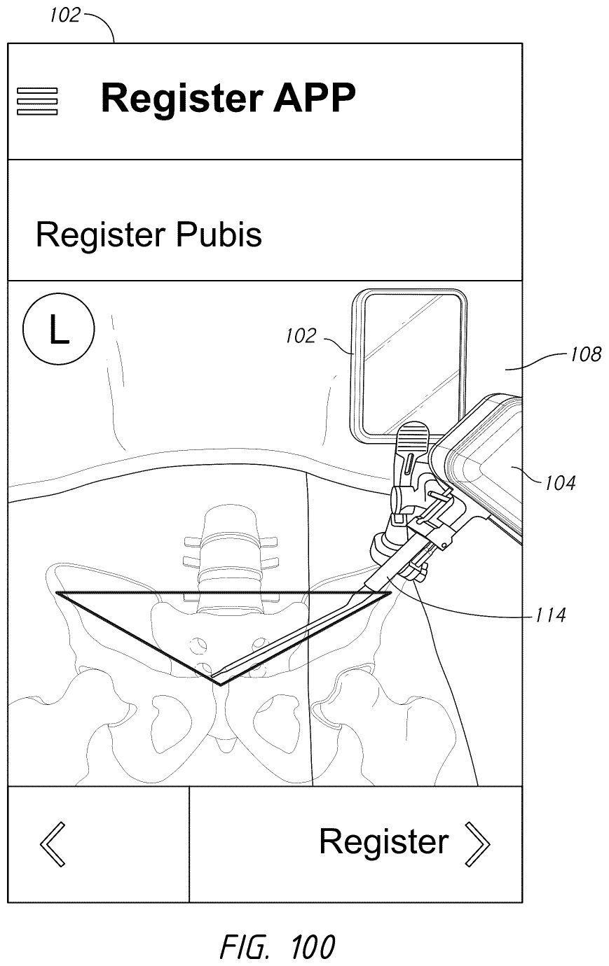

The method can include registering the patient's anterior pelvic plane (APP). The method can include positioning the tip of the registration probe 114 in contact with a point. The patient can be positioned supine. The registration probe 114 can be coupled to the reference sensor device 104. The method can include pressing register on the graphical user interface 108 of the navigation device 102. The method can include positioning the tip of the registration probe 114 in contact with the Ipsilateral ASIS as shown in FIG. 98. The method can include pressing register to register the Ipsilateral ASIS on the graphical user interface 108 of the navigation device 102. The method can include positioning the tip of the registration probe 114 in contact with the Contralateral ASIS as shown in FIG. 99. The method can include pressing register to register the Contralateral ASIS on the graphical user interface 108 of the navigation device 102. The method can include positioning the tip of the registration probe 114 in contact with the Pubis as shown in FIG. 100. The method can include pressing register to register the Pubis on the graphical user interface 108 of the navigation device 102. The method can include pressing register to register the position, orientation, and/or rotation of the reference sensor device 104 when the registration probe 114 contacts a point. FIGS. 98-100 illustrate the graphical user interface 108 of the navigation device 102 providing directions to the user related to these steps. The graphical user interface 108 of the navigation device 102 can illustrate the navigation device 102 with on-screen graphics.

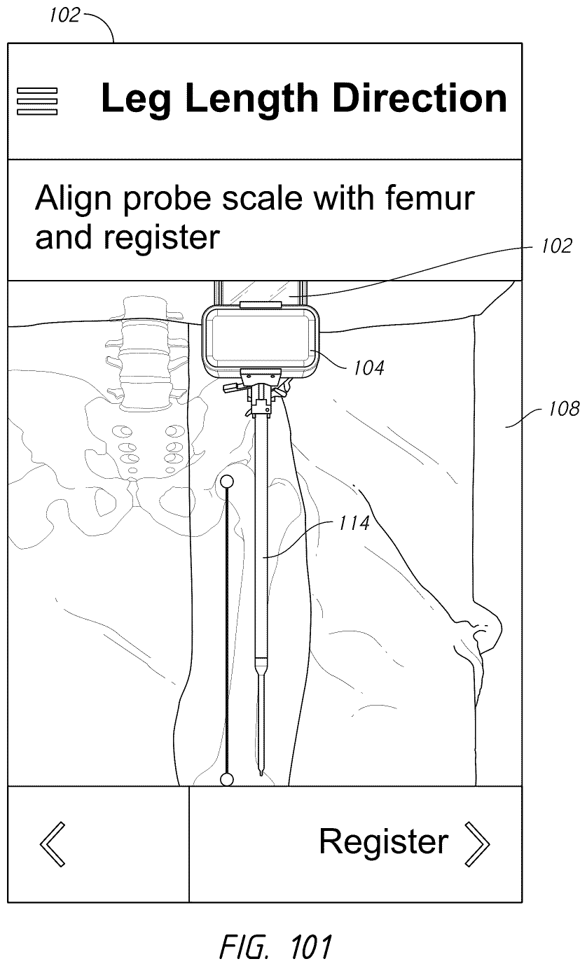

The method can include registering leg length direction. The method can include registering leg length direction by aligning the registration probe 114 with the long axis of the femur as shown in FIG. 101. The registration probe 114 can be coupled to the reference sensor device 104. The method can include aligning the marking 179 of the registration probe 114 with the patient's femur. The patient can be positioned supine. The method can include pressing register to register the leg length direction on the graphical user interface 108 of the navigation device 102. FIG. 101 illustrates the graphical user interface 108 of the navigation device 102 providing directions to the user related to this step.

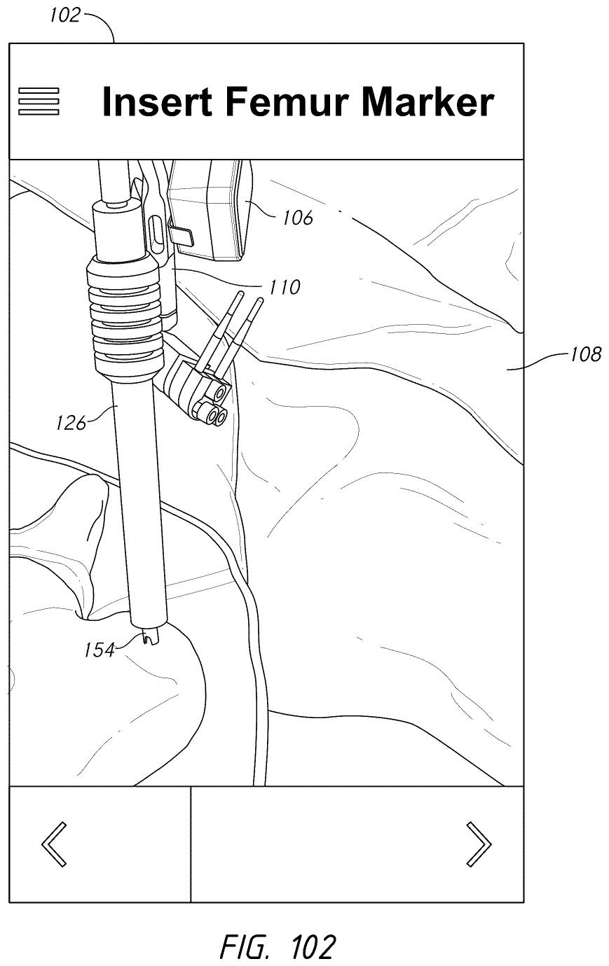

The method can include inserting the femur registration marker 154. The method can include utilizing the holding sleeve 126 and the hex driver 124. The hex driver 124 can be disposed within the holding sleeve 126. The femur sensor device 106 can remain coupled to the pelvic bracket 110 coupled to the pelvis. The method can include rotating the hex driver 124 to rotate the femur registration marker 154 into bone. The method can include inserting the femur registration marker 154 into the greater trochanter. The method can include pressing the advance arrow on the graphical user interface 108 of the navigation device 102 when the femur registration marker 154 is inserted. FIG. 102 illustrates the graphical user interface 108 of the navigation device 102 providing directions to the user related to this step.

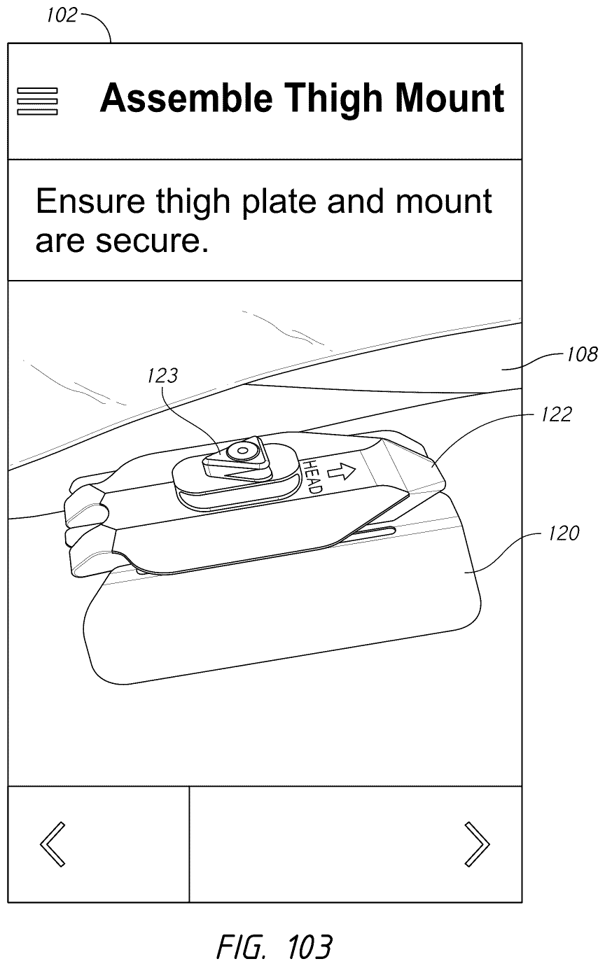

The method can include assembling the thigh mount 122 on the distal thigh. The method can include wrapping a portion of the distal thigh in Coban™. The method can include placing the thigh plate 120 onto the Coban™. The method can include securing the thigh plate 120 to the distal thigh using one or more strips of Ioban™. The method can include coupling the thigh mount 122 to the thigh plate 120. In some embodiments, the thigh mount 122 can couple to the thigh plate 120 in a single orientation. The user can ensure that the thigh plate 120 and the thigh mount 122 are secure. The user can ensure that the arrow of the thigh mount 122 points toward the head of the patient. The coupler 123 of the thigh mount 122 can allow for coupling with the femur sensor device 106. FIG. 103 illustrates the graphical user interface 108 of the navigation device 102 providing directions to the user related to this step.

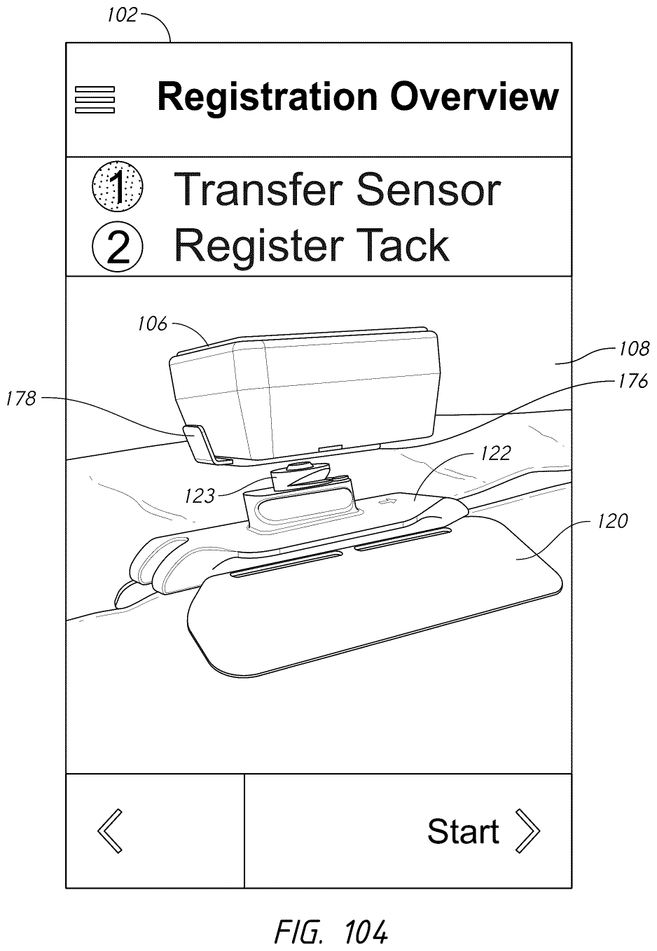

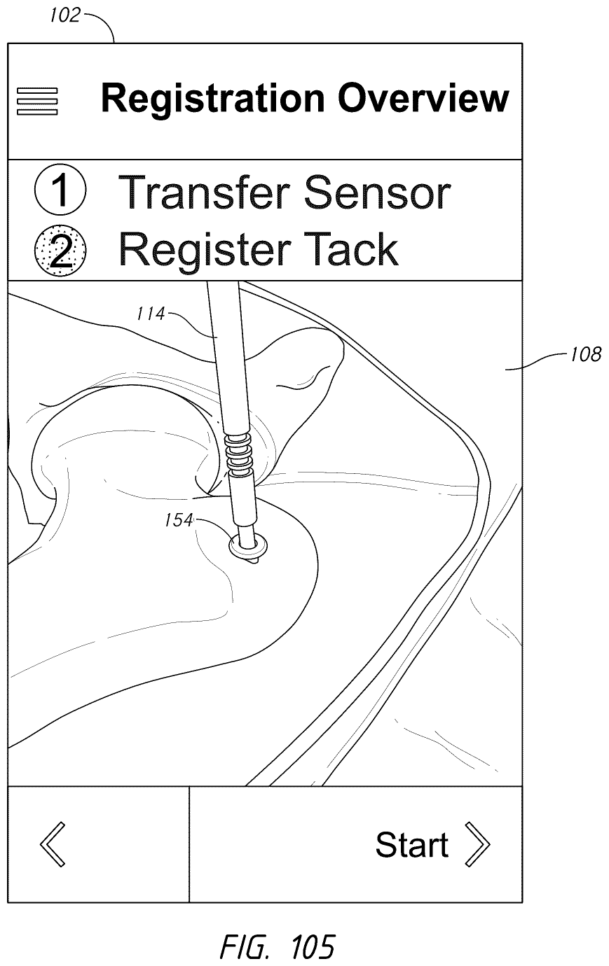

The method can include a registration overview. The method can include transferring the femur sensor device 106. The femur sensor device 106 can be transferred from the pelvic bracket 110 mounted to the pelvis of the patient to the thigh mount 122. The coupler 123 of the thigh mount 122 can couple with the receiver 176 the femur sensor device 106. The lever 178 of the femur sensor device 106 can point toward the feet of the patient. In some embodiments, the coupler 123 of the thigh mount 122 can couple to the femur sensor device 106 in a single orientation. The method can include tapping the start button on the graphical user interface 108 of the navigation device 102 to begin registration. FIG. 104 illustrates the graphical user interface 108 of the navigation device 102 providing directions to the user related to this step.

The method can include a registration overview. The method can include registering the femur registration marker 154. The method can include tapping the start button on the graphical user interface 108 of the navigation device 102 to begin registration. FIG. 105 illustrates the graphical user interface 108 of the navigation device 102 providing directions to the user related to this step.

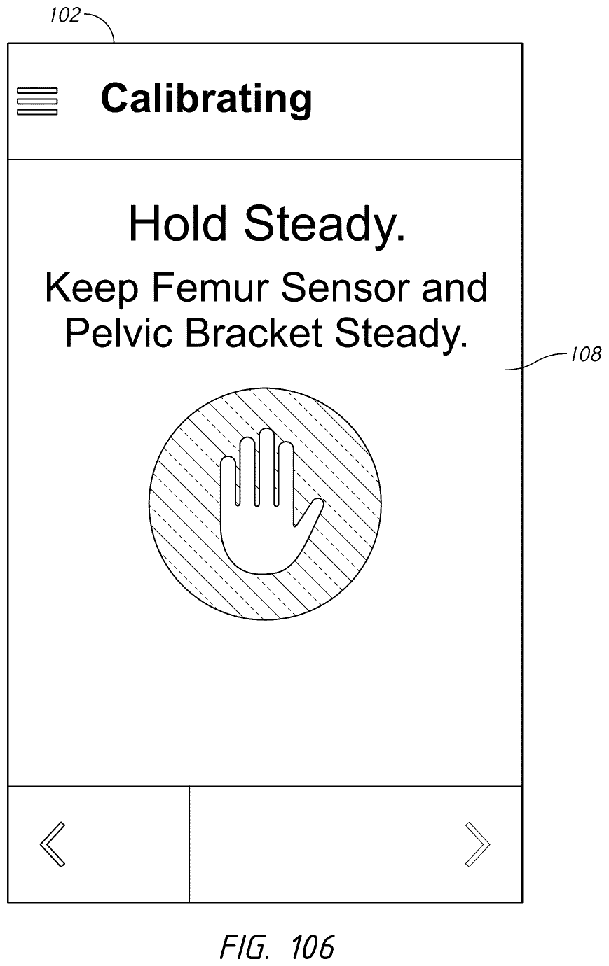

The method can include calibrating. The user can hold the femur sensor device 106 steady. The user can hold the pelvic bracket 110 steady. The method can include displaying a red stop icon on the graphical user interface 108 of the navigation device 102. FIG. 106 illustrates the graphical user interface 108 of the navigation device 102 providing directions to the user related to this step.

The method can include transferring the femur sensor device 106. The femur sensor device 106 can be transferred from the pelvic bracket 110 mounted to the pelvis of the patient to the thigh mount 122.

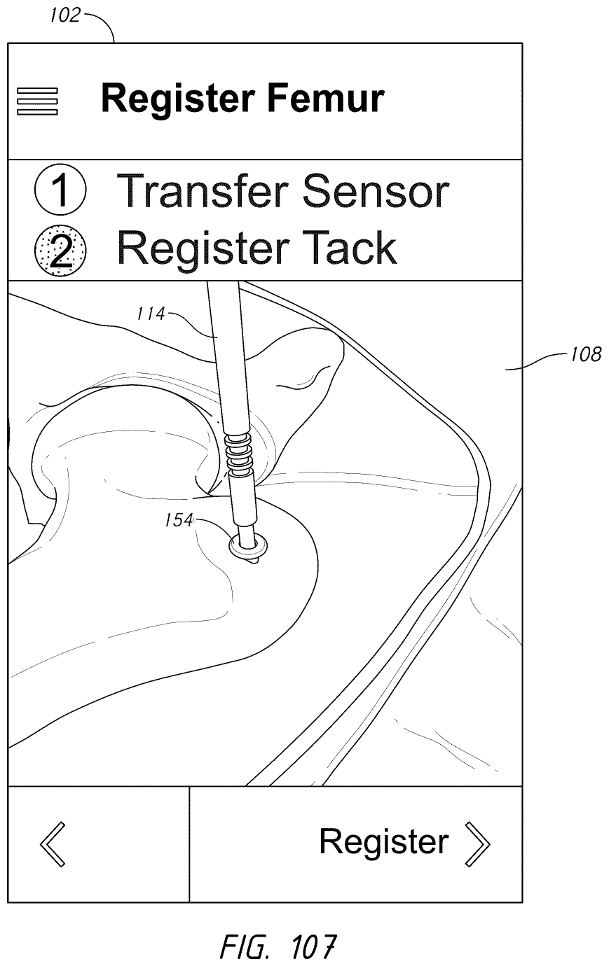

The method can include registering the femur registration marker 154. The method can include registering the femur registration marker 154 before the cup is placed. The method can include inserting the registration probe 114 into the head of the femur registration marker 154. The reference sensor device 106 can be coupled to the registration probe 114. The registration probe 114 and the reference sensor device 104 can be coupled to the pelvic bracket 110 coupled to the pelvis of the patient. The navigation device 102 can be coupled to the pelvic bracket 110 coupled to the pelvis of the patient. The femur sensor device 106 can be coupled to the thigh mount 122 as shown in FIG. 104. The method can include pressing register to register the femur registration marker 154 on the graphical user interface 108 of the navigation device 102. The method can include pressing register to register the position, orientation, and/or rotation of the reference sensor device 104 when the registration probe 114 contacts the femur registration marker 154. The method can include pressing register to register the orientation and/or rotation of the femur sensor device 106 when the registration probe 114 contacts the femur registration marker 154. The method can include press register on the graphical user interface 108 of the navigation device 102 when the step is completed. The baseline rotation and/or orientation of the femur can be registered by the femur sensor device 106. The baseline position, rotation and/or orientation of the femur registration marker 154 can be registered by the reference sensor device 104. These baseline measurements of the femur registration marker 154 can be stored by the navigation device 102. FIG. 107 illustrates the graphical user interface 108 of the navigation device 102 providing directions to the user related to this step.

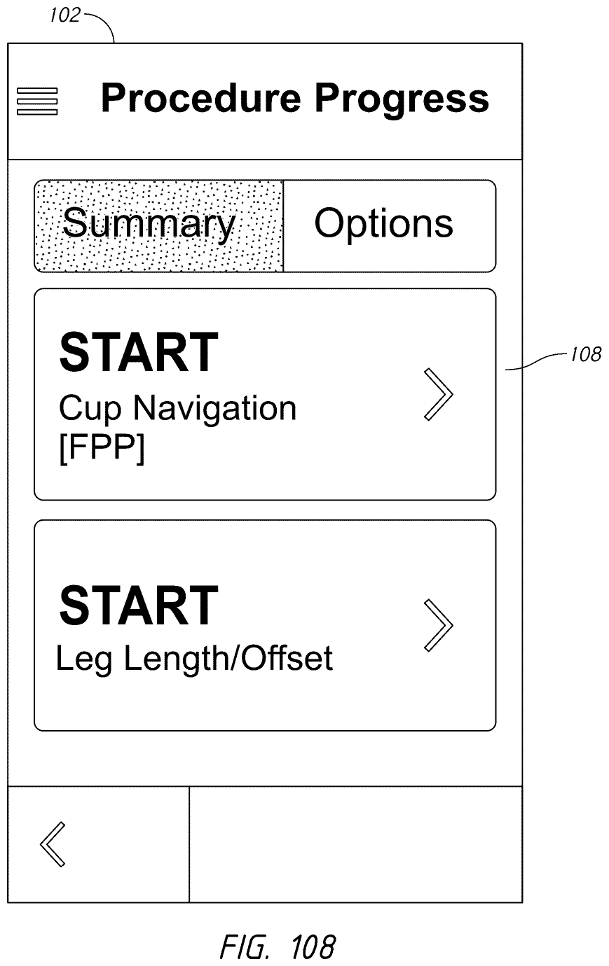

The method can include providing a summary of progress of the surgical procedure. The user can select to start cup navigation. The user can select options, such as selecting an alternative reference frame. The user can select to start leg length and/or joint offset measurements. FIG. 108 illustrates the graphical user interface 108 of the navigation device 102 providing the summary to the user related to this step.

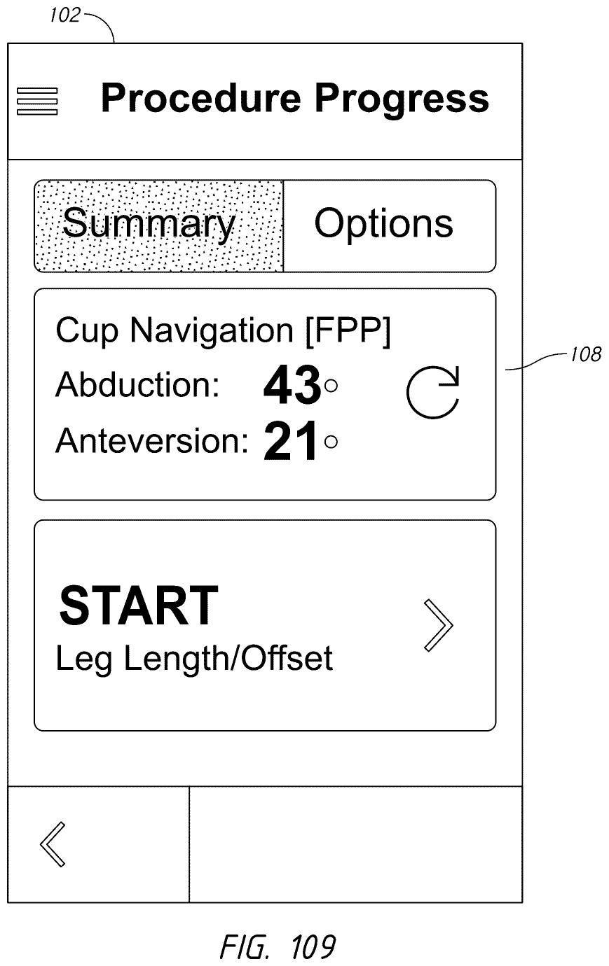

The method can include providing a summary of progress of the surgical procedure after cup placement. The graphical user interface 108 of the navigation device 102 can display the navigated angles. The graphical user interface 108 of the navigation device 102 can display abduction and anteversion angles. The graphical user interface 108 of the navigation device 102 can display the selected reference frame. The user can select to complete the navigation again by selecting the repeat icon. The user can select to start leg length and/or joint offset measurements. FIG. 109 illustrates the graphical user interface 108 of the navigation device 102 providing the summary to the user related to this step.

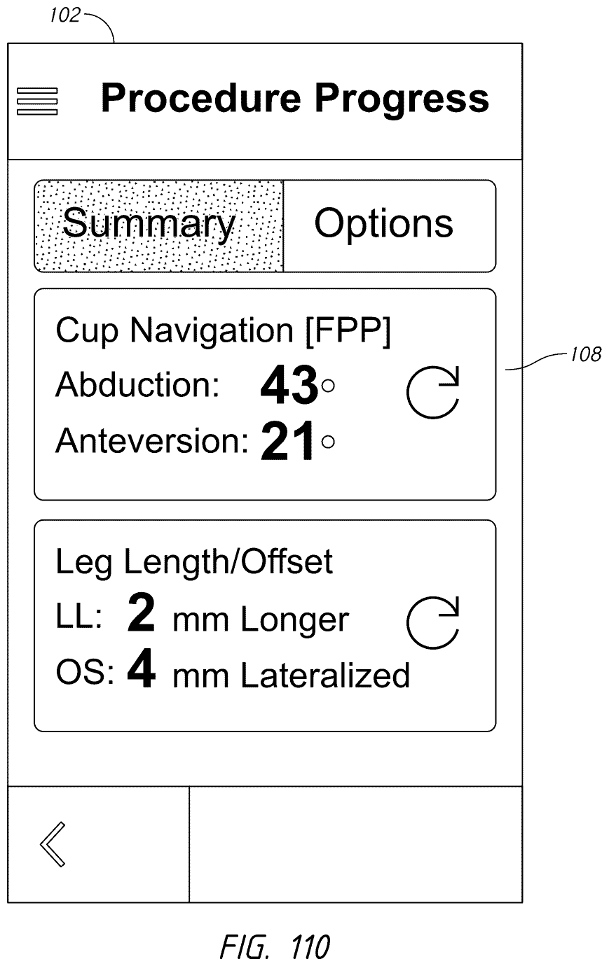

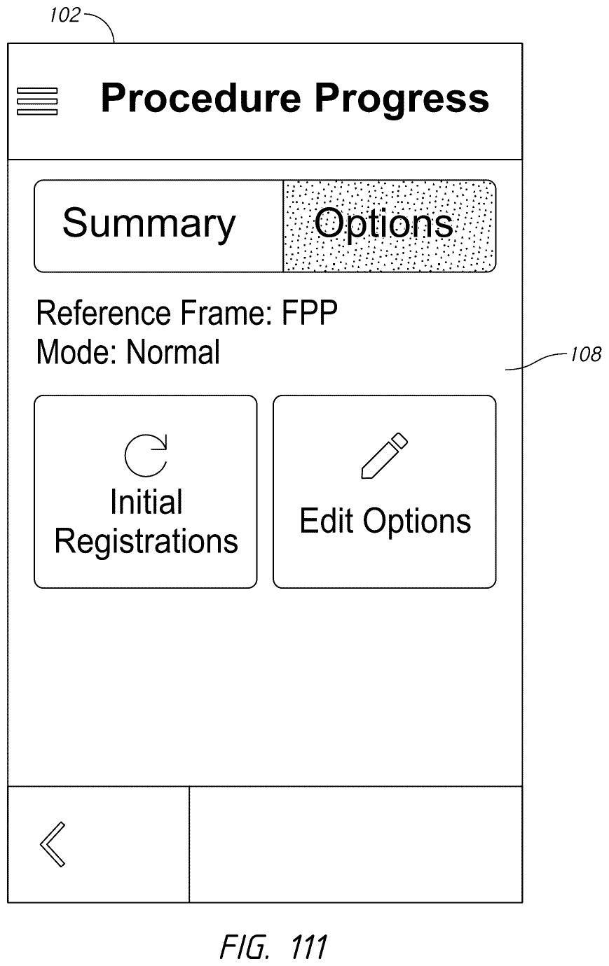

The method can include providing a summary of progress of the surgical procedure after leg length and/or joint offset measurements. The graphical user interface 108 of the navigation device 102 can display the navigated angles. The graphical user interface 108 of the navigation device 102 can display abduction and anteversion angles. The graphical user interface 108 of the navigation device 102 can display the selected reference frame. The user can select to complete the navigation again by selecting the repeat icon. The graphical user interface 108 of the navigation device 102 can display the change in leg length. The graphical user interface 108 of the navigation device 102 can display the direction in the change in leg length, e.g., longer or shorter. The graphical user interface 108 of the navigation device 102 can display the change in joint offset. The graphical user interface 108 of the navigation device 102 can display the direction in the change in joint offset, e.g., lateralized or medialized. The user can select to complete the leg length and joint offset measurements again by selecting the repeat icon. FIG. 110 illustrates the graphical user interface 108 of the navigation device 102 providing the summary to the user related to this step.

The method can include providing a summary of progress of the surgical procedure. The user can select options. The user can select the reference frame. The user can select the mode, either regular or advanced mode. The user can repeat initial registrations. The user can edit options. FIG. 111 illustrates the graphical user interface 108 of the navigation device 102 providing options to the user related to this step.

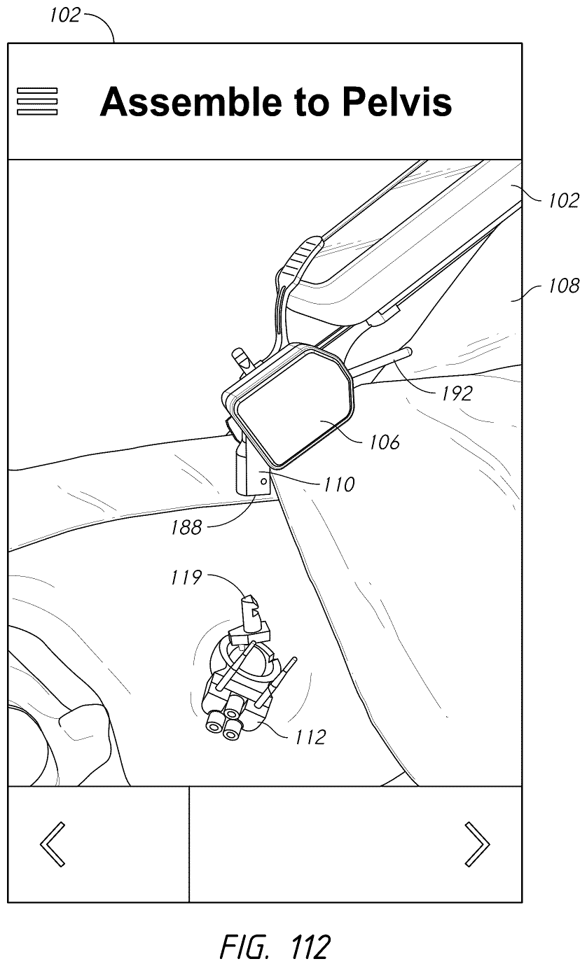

The method can include assembling the pelvic bracket 110 to the pelvic base 112. The navigation device 102 and the femur sensor device 106 can be coupled to the pelvic bracket 110. The pelvic bracket 110 can be lowered onto the pelvic base 112. The pelvic base 112 can include the coupler 119. The method can include depressing the lever 192 of the pelvic bracket 110. The method can include lowering the receiver 188 of the pelvic bracket relative to the coupler 119 the pelvic base 112. The method can include releasing the lever 192. FIG. 112 illustrates the graphical user interface 108 of the navigation device 102 providing directions to the user related to this step.

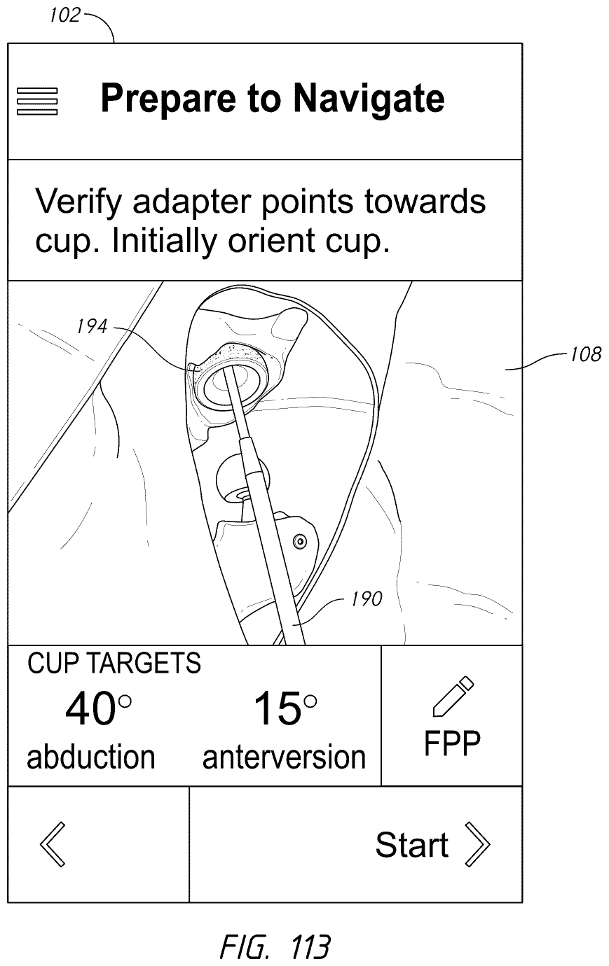

The method can include preparing to navigate. The method can include coupling the impactor adapter clip 144 to the impactor 190. The method can include coupling the cup 194 to the impactor 190. The user can verify that the impactor adapter clip 144 points toward the cup 194. The impactor adapter clip 144 can include the coupler 189. The user can verify that the coupler 189 of the impactor adapter clip 144 points toward the cup 194. The user can initially orient the cup 194 relative to the acetabulum. The graphical user interface 108 of the navigation device 102 can display target angles. The graphical user interface 108 of the navigation device 102 can display the reference frame for navigation. The user can press start on the graphical user interface 108 of the navigation device 102 when ready to begin navigation. FIG. 113 illustrates the graphical user interface 108 of the navigation device 102 providing directions to the user related to this step.

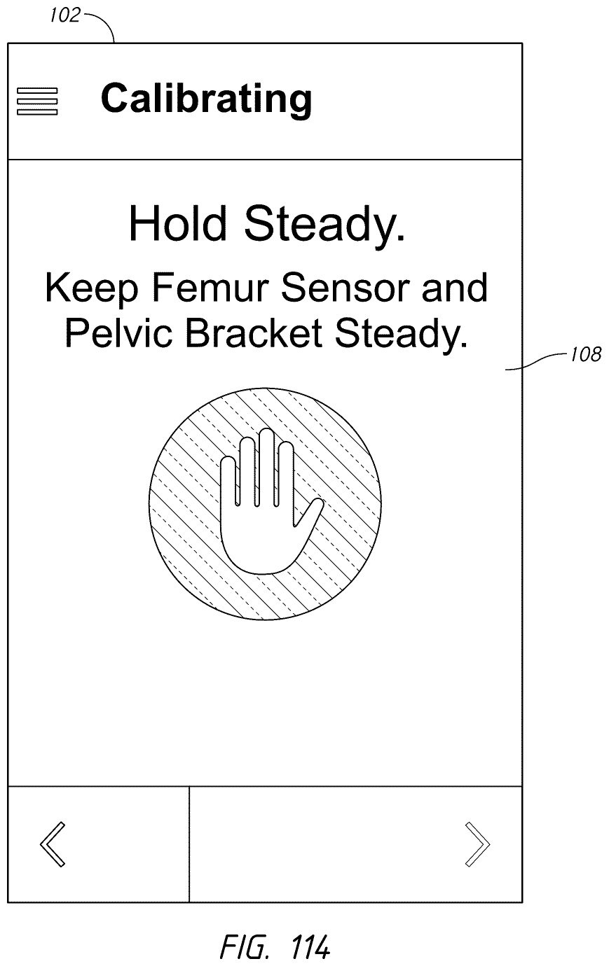

The method can include calibrating. The user can hold the femur sensor device 106 steady. The user can hold the pelvic bracket 110 steady. In some methods, the femur sensor device 106 is coupled to the pelvic bracket 110 and the pelvic bracket 110 is coupled to the pelvis of the patient. In some methods, the femur sensor device 106 is calibrated relative to the navigation device 102. In some methods, the femur sensor device 106 is in a known orientation relative to the navigation device 102. The method can include displaying a red stop icon on the graphical user interface 108 of the navigation device 102. FIG. 114 illustrates the graphical user interface 108 of the navigation device 102 providing directions to the user related to this step.

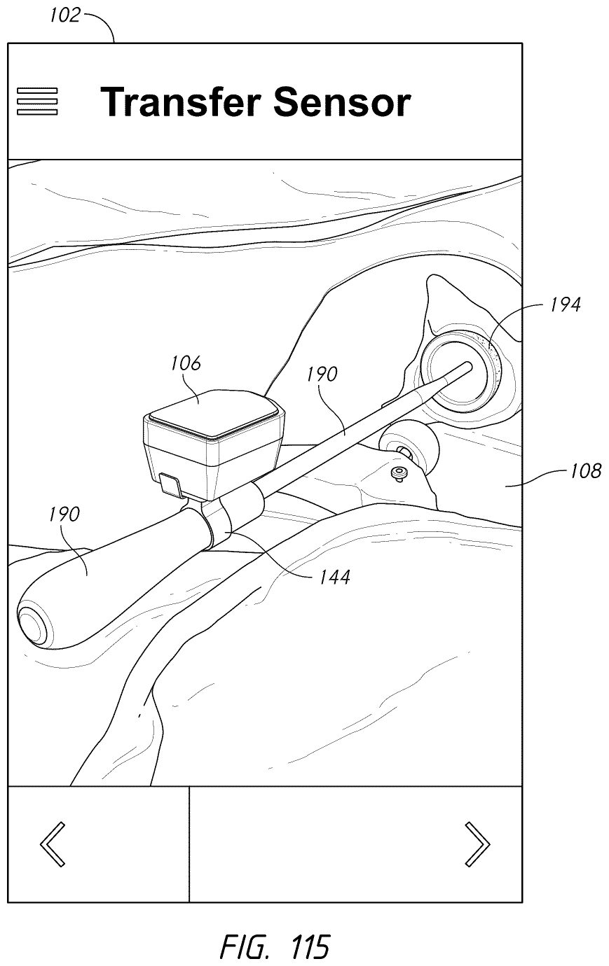

The method can include transferring the femur sensor device 106. The femur sensor device 106 can be transferred from the pelvic bracket 110 coupled to the pelvis of the patient to the impactor adapter clip 144 coupled to the impactor 190. The cup 194 can be initially oriented within the acetabulum before the femur sensor device 106 is transferred. The lever 178 on the femur sensor device 106 can point toward the foot of the patient. In some embodiments, the impactor adapter clip 144 and the femur sensor device 106 can coupled in a single orientation. The user can press the advanced arrow on the graphical user interface 108 of the navigation device 102 when the femur sensor device 106 is transferred. FIG. 115 illustrates the graphical user interface 108 of the navigation device 102 providing directions to the user related to this step.

The method can include navigating the cup 194. The graphical user interface 108 of the navigation device 102 can display the selected reference frame. The graphical user interface 108 of the navigation device 102 can allow the user to select an alternative reference frame. The graphical user interface 108 of the navigation device 102 can display angles as the impactor 190 is moved. The graphical user interface 108 of the navigation device 102 can display abduction and anteversion angles relative to the selected reference frame. The graphical user interface 108 of the navigation device 102 can display the leg that is being operated upon, either left or right.

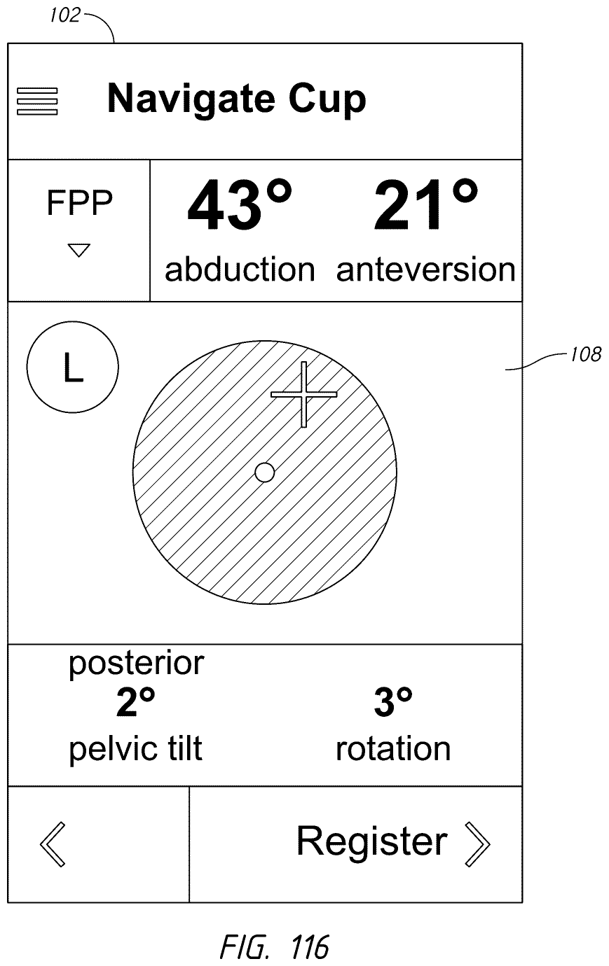

The graphical user interface 108 of the navigation device 102 can provide a target. The target can be based on target angles inputted to the navigation device 102. The center of the target can indicate when the impactor 190 is navigated to target abduction and anteversion angles. The cross-hair marker can move as the impactor 190 moves. The cross-hair marker can move to assist the user in visualizing the navigated angles relative to the target angles. The graphical user interface 108 of the navigation device 102 can display a measurement of pelvic tilt. The graphical user interface 108 of the navigation device 102 can display a direction of a measurement of pelvic tilt. The graphical user interface 108 of the navigation device 102 can display a measurement of rotation. The user can press the register on the graphical user interface 108 of the navigation device 102 when the impactor 190 is positioned. FIG. 116 illustrates the graphical user interface 108 of the navigation device 102 providing directions to the user related to this step.

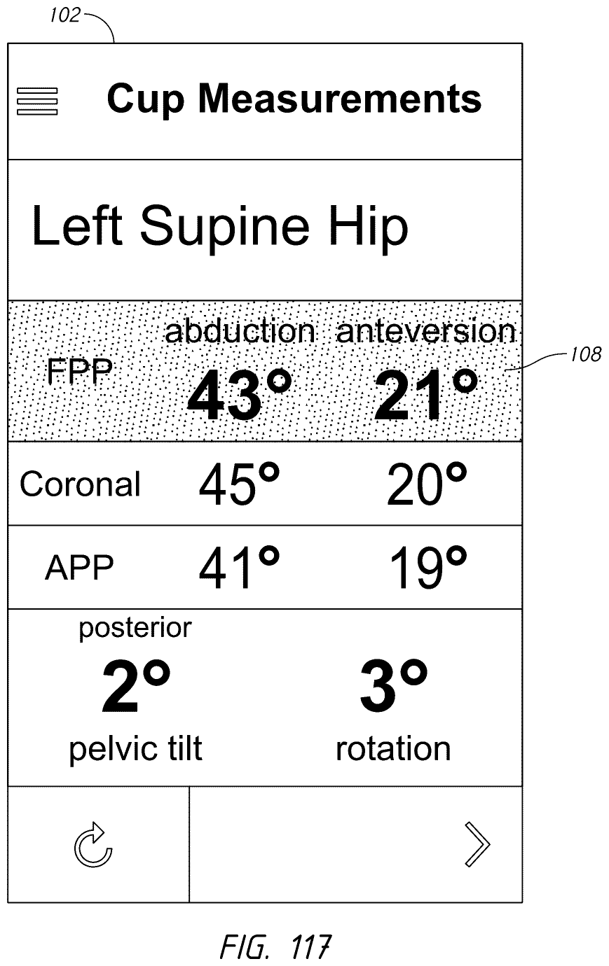

The method can include providing cup measurements. The graphical user interface 108 of the navigation device 102 can display the abduction and anteversion angles relative to the functional pelvic plane. The graphical user interface 108 of the navigation device 102 can display the abduction and anteversion angles relative to the coronal plane. The graphical user interface 108 of the navigation device 102 can display the abduction and anteversion angles relative to the anterior pelvic plane. The graphical user interface 108 of the navigation device 102 can display pelvic tilt. The graphical user interface 108 of the navigation device 102 can display rotation. The user can press the advance arrow to proceed. The user can press the repeat icon to repeat navigation. FIG. 117 illustrates the graphical user interface 108 of the navigation device 102 providing directions to the user related to this step.

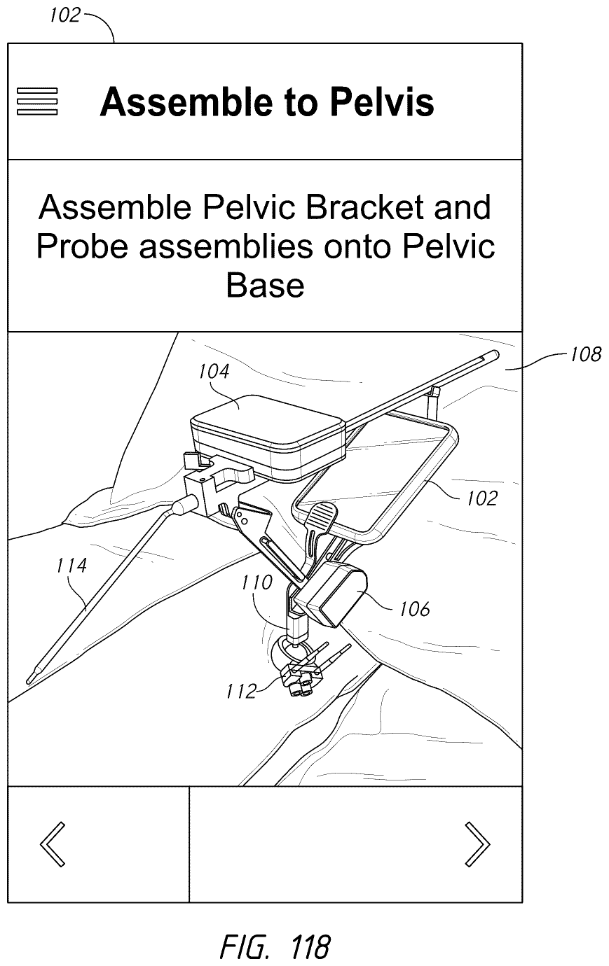

The method can include assembling the pelvic bracket 110 to the pelvic base 112. The navigation device 102 and the femur sensor device 106 can be coupled to the pelvic bracket 110. The method can include assembling the registration probe 114 to the pelvic bracket 100. The method can include assembling the reference sensor device 106 to the registration probe 114. FIG. 118 illustrates the graphical user interface 108 of the navigation device 102 providing directions to the user related to this step.

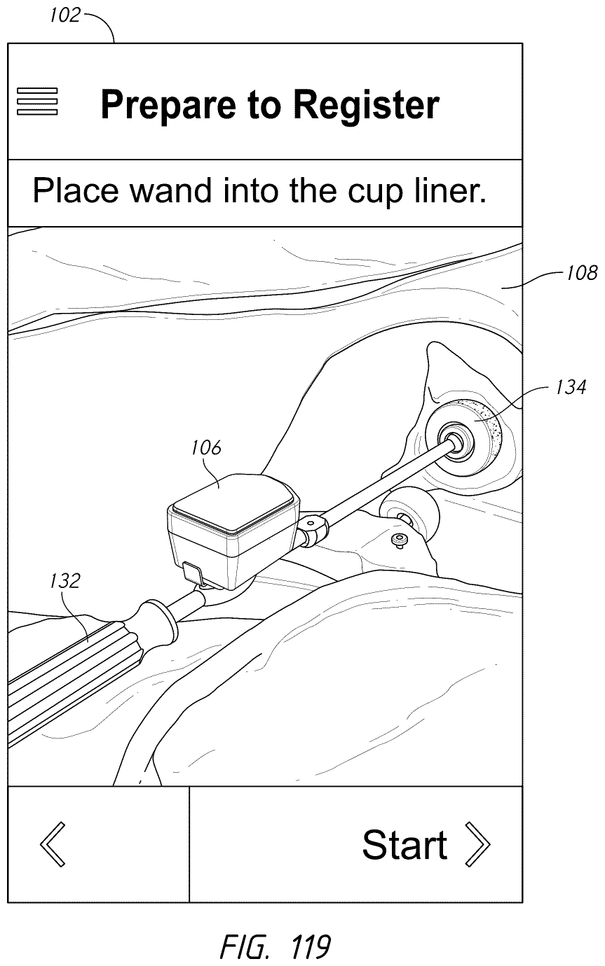

The method can include preparing to register the center of rotation. The method can include placing the liner registration trial 134 of the registration wand 132 into the liner 196 of the cup 194. The femur sensor device 106 can be couped to the registration wand 132. The user can press the start on the graphical user interface 108 of the navigation device 102 when ready to begin registering the center of rotation. FIG. 119 illustrates the graphical user interface 108 of the navigation device 102 providing directions to the user related to this step.

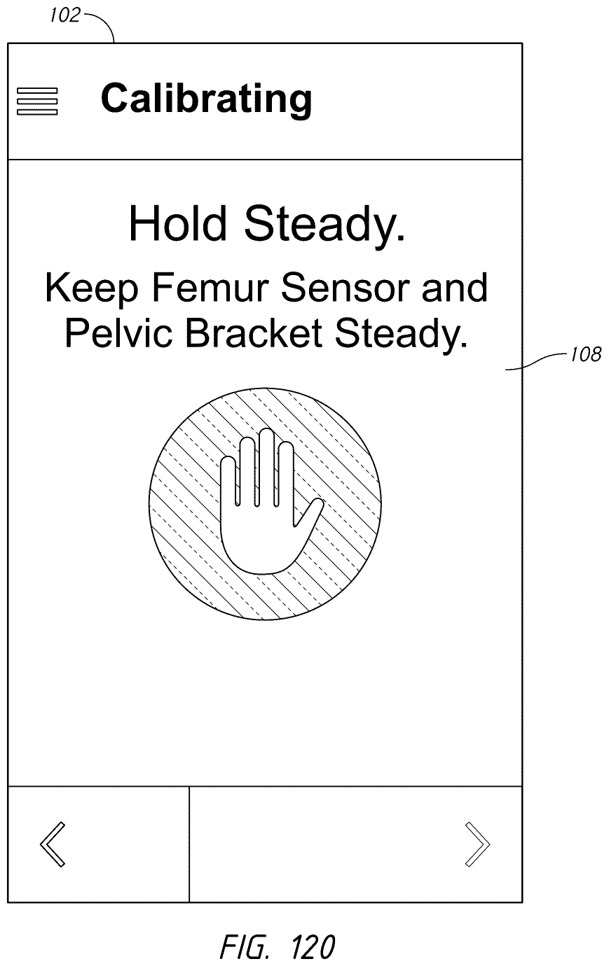

The method can include calibrating. The user can hold the femur sensor device 106 steady. The user can hold the pelvic bracket 110 steady. The method can include displaying a red stop icon on the graphical user interface 108 of the navigation device 102. FIG. 120 illustrates the graphical user interface 108 of the navigation device 102 providing directions to the user related to this step.

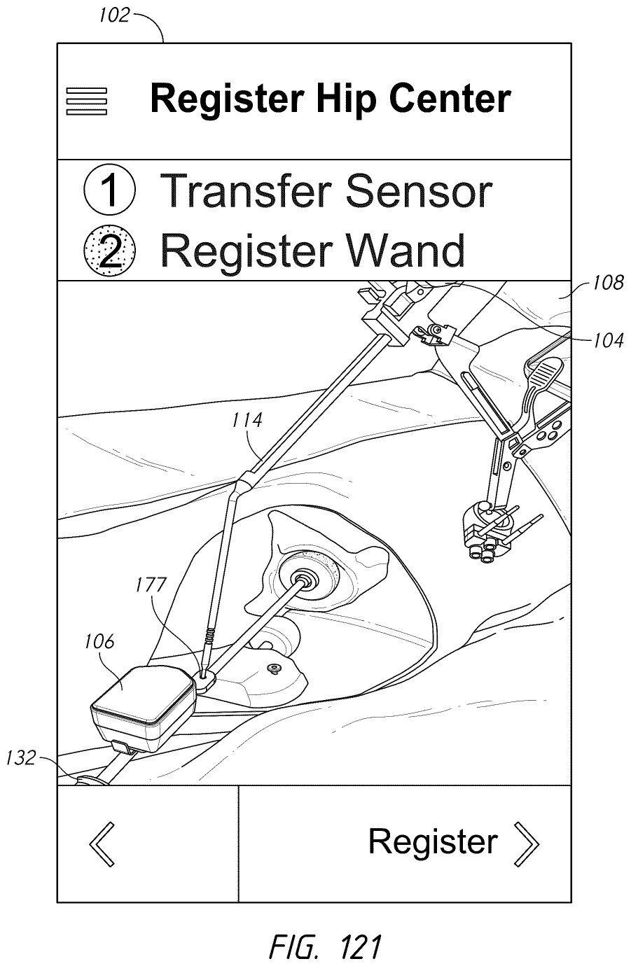

The method can include transferring the femur sensor device 106 from the pelvic bracket 110 to the registration wand 132. The method can include placing the liner registration trial 134 of the registration wand 132 into the liner 196 of the cup 194. The method can include registering the registration wand 132. The method can include registering a point with the registration probe 114. The reference sensor device 104 can be coupled to the registration probe 114. The method can include inserting the tip of the registration probe 114 into the divot 177 of the registration wand 132. The method can include pressing register on the graphical user interface 108 of the navigation device 102 to register the registration wand 132. FIG. 121 illustrates the graphical user interface 108 of the navigation device 102 providing directions to the user related to this step.

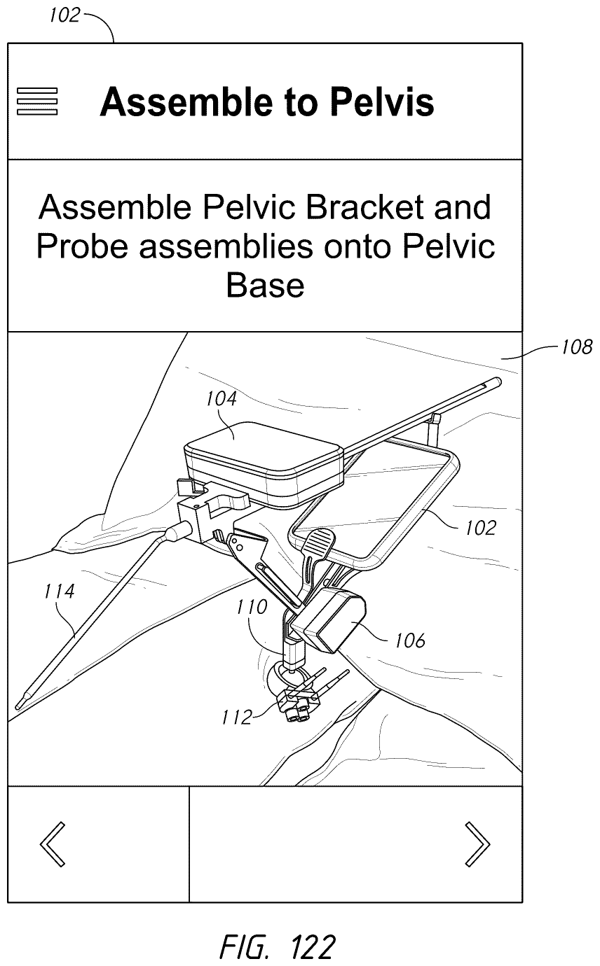

The method can include assembling the pelvic bracket 110 to the pelvic base 112. The navigation device 102 and the femur sensor device 106 can be coupled to the pelvic bracket 110. The method can include assembling the registration probe 114 to the pelvic bracket 100. The method can include assembling the reference sensor device 106 to the registration probe 114. FIG. 122 illustrates the graphical user interface 108 of the navigation device 102 providing directions to the user related to this step.

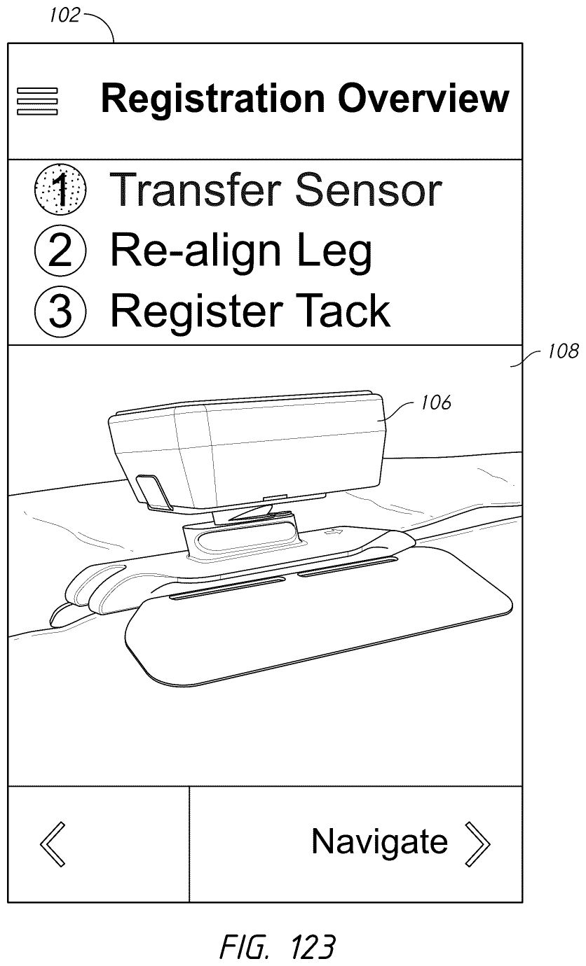

The method can include an overview of registration. The method can include transferring the femur sensor device 106. FIG. 123 illustrates the graphical user interface 108 of the navigation device 102 providing directions to the user related to this step.

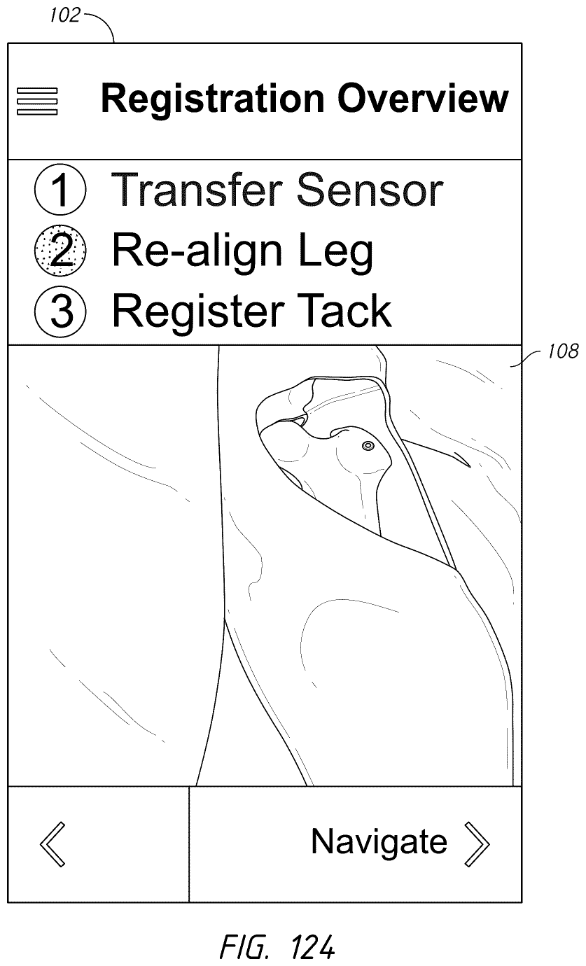

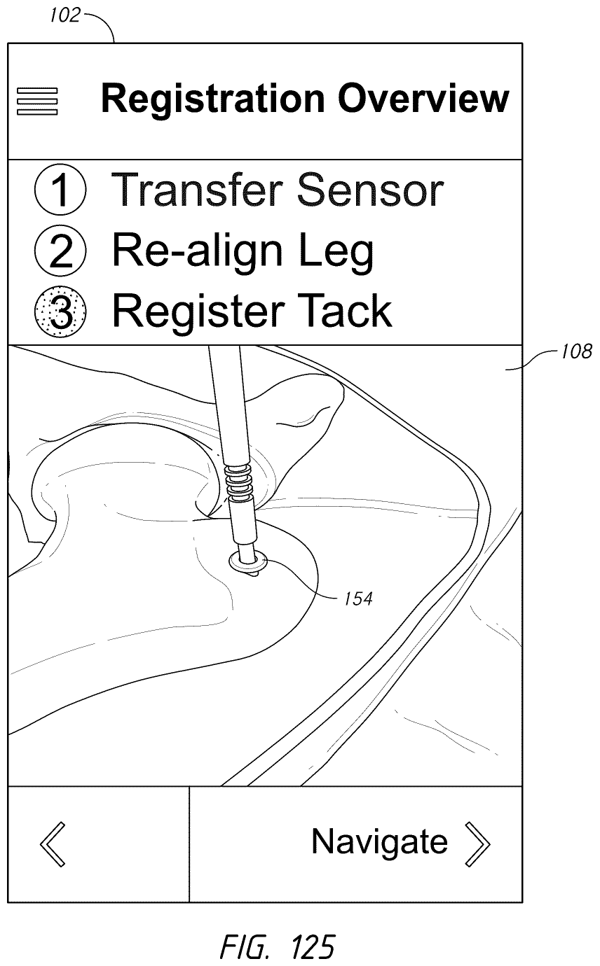

The method can include an overview of registration. The method can include realigning the leg of the patient. FIG. 124 illustrates the graphical user interface 108 of the navigation device 102 providing directions to the user related to this step.

The method can include an overview of registration. The method can include registering the femur registration marker 154. FIG. 125 illustrates the graphical user interface 108 of the navigation device 102 providing directions to the user related to this step.

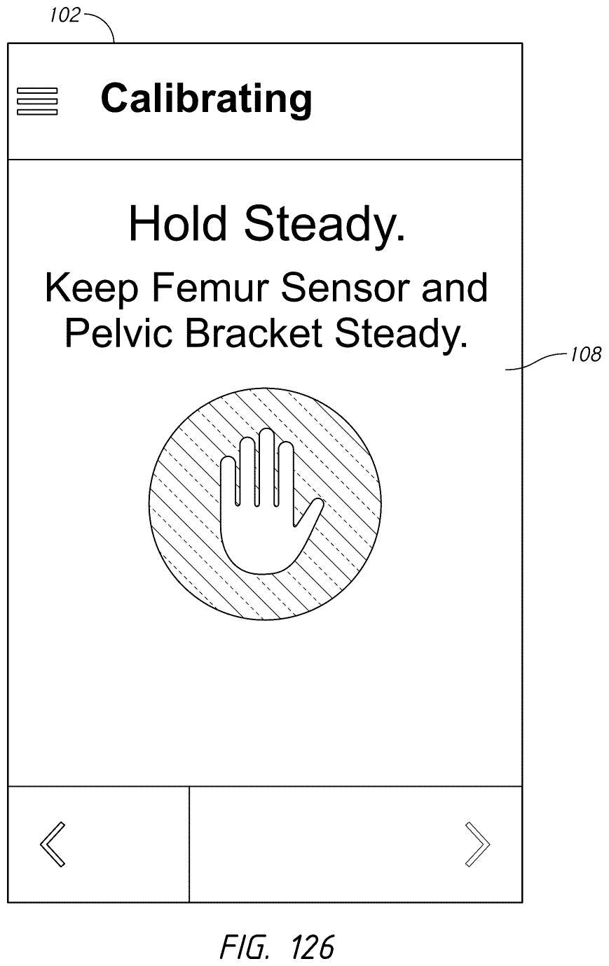

The method can include calibrating. The user can hold the femur sensor device 106 steady. The user can hold the pelvic bracket 110 steady. The user can hold the registration probe 114 steady. The method can include displaying a red stop icon on the graphical user interface 108 of the navigation device 102. FIG. 126 illustrates the graphical user interface 108 of the navigation device 102 providing directions to the user related to this step.

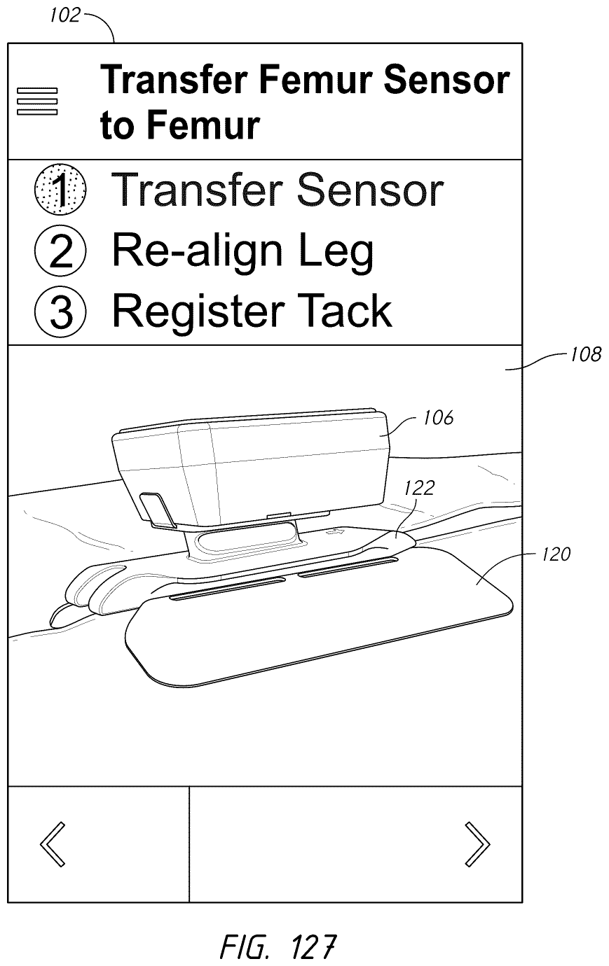

The method can include transferring the femur sensor device 106. The femur sensor device 106 can be transferred from the pelvic bracket 110 mounted to the pelvis of the patient to the thigh mount 122. The thigh mount 122 can be coupled to the thigh plate 120. The thigh plate 120 can be coupled to the distal thigh of the patient. The method can include tapping the advance button on the graphical user interface 108 of the navigation device 102 when the femur sensor device 106 is transferred. FIG. 127 illustrates the graphical user interface 108 of the navigation device 102 providing directions to the user related to this step.

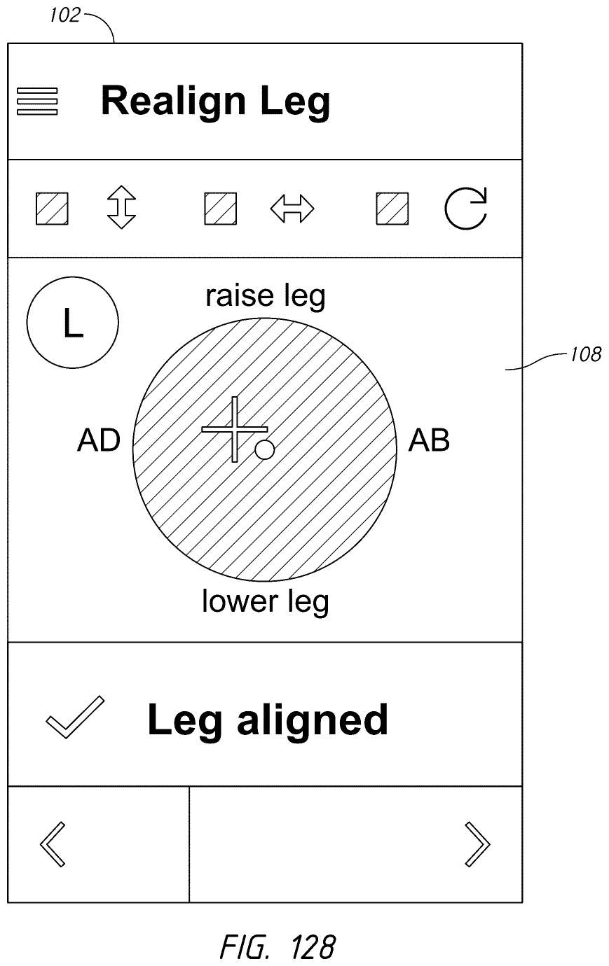

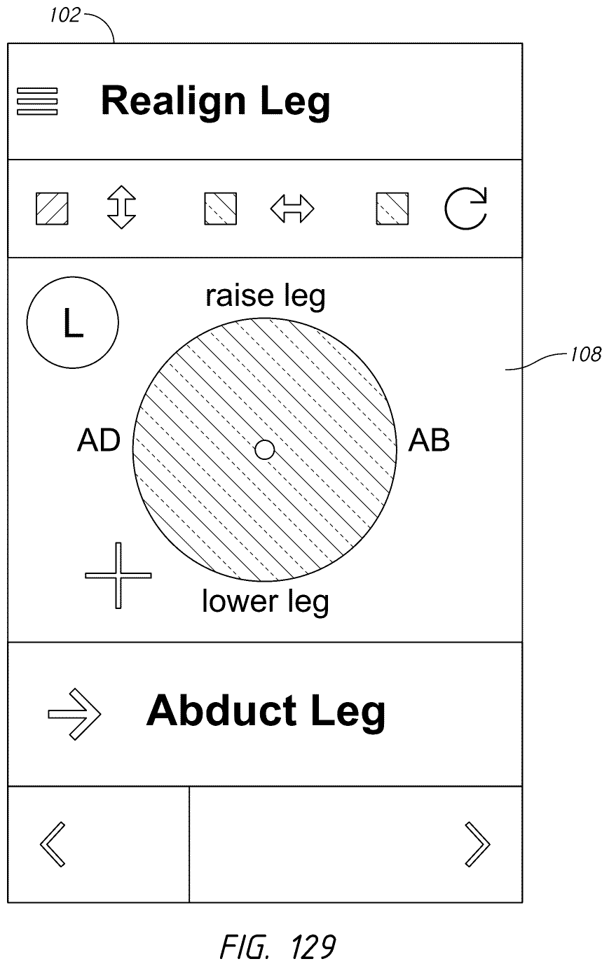

The method can include realigning the leg of the patient. The method can include realigning the leg after the cup is placed. The method can include realigning the femur relative to the pelvis. The graphical user interface 108 of the navigation device 102 can provide a target. The target can be based on the baseline rotation and/or orientation of the femur before the cup was placed. The target can be based on the baseline rotation and/or orientation of the femur as registered by the femur sensor device 106. The center of the target can indicate when the femur is positioned within an acceptable range of the baseline position. The cross-hair marker can move as the femur moves. The cross-hair marker can move to assist the user in visualizing the position of the femur relative to the baseline or initial position. The graphical user interface 108 of the navigation device 102 can provide instructions to the user to reposition the femur. The user can raise or lower the femur based on instructions. The user can move the femur in abduction (AB) or adduction (AD) based on instructions. The user can press the advance button on the graphical user interface 108 of the navigation device 102 when the femur is positioned. FIG. 128 illustrates the graphical user interface 108 of the navigation device 102 providing directions to the user related to this step. The femur is positioned within an acceptable range of the baseline measurement in FIG. 128. FIG. 129 illustrates the graphical user interface 108 of the navigation device 102 providing directions to the user related to this step. The femur is positioned outside of an acceptable range of the baseline measurement in FIG. 129.

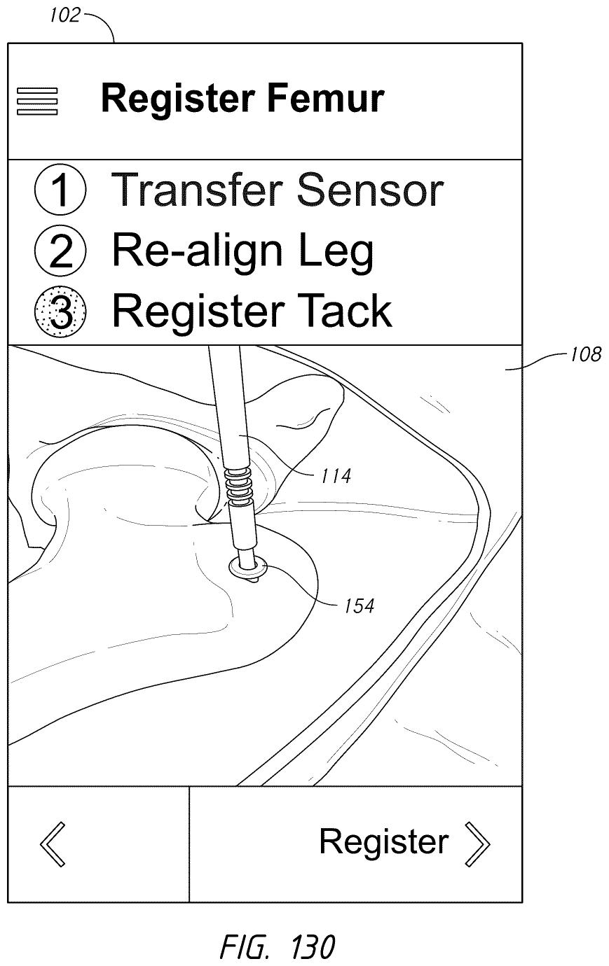

The method can include registering the femur registration marker 154. The method can include registering the femur registration marker 154 after the cup is placed. The method can include inserting the registration probe 114 into the head of the femur registration marker 154. The method can include pressing register on the graphical user interface 108 of the navigation device 102 when the registration probe 114 is contacting the registration marker 154. The orientation and/or rotation of the femur can be registered by the femur sensor device 106 after cup placement. The position, rotation and/or orientation of the femur registration marker 154 can be registered by the reference sensor device 104 after cup placement. These later measurements of the femur registration marker 154 can be stored by the navigation device 102. FIG. 130 illustrates the graphical user interface 108 of the navigation device 102 providing directions to the user related to this step.

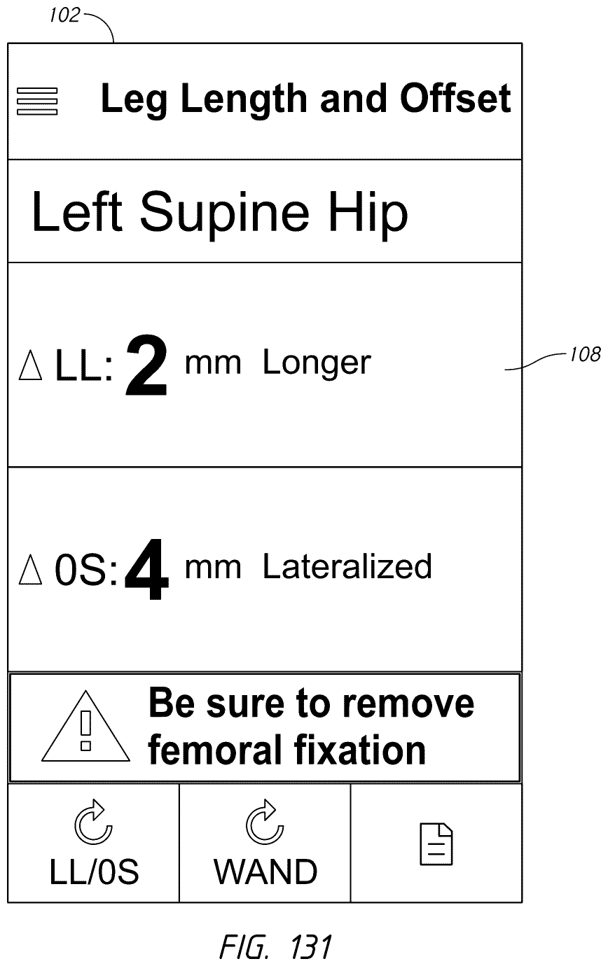

The method can include providing a summary of progress of the surgical procedure. The method can include providing a summary of changes in leg length and/or joint offset. The graphical user interface 108 of the navigation device 102 can display the change in leg length. The graphical user interface 108 of the navigation device 102 can display the direction in the change in leg length, e.g., longer or shorter. The graphical user interface 108 of the navigation device 102 can display the change in joint offset. The graphical user interface 108 of the navigation device 102 can display the direction in the change in joint offset, e.g., lateralized or medialized. The user can select to complete the leg length and joint offset measurements again by selecting the repeat icon. The user can select to complete the wand registration again by selecting the repeat icon. The user can select to save the summary by selecting the report icon. FIG. 131 illustrates the graphical user interface 108 of the navigation device 102 providing directions to the user related to this step.

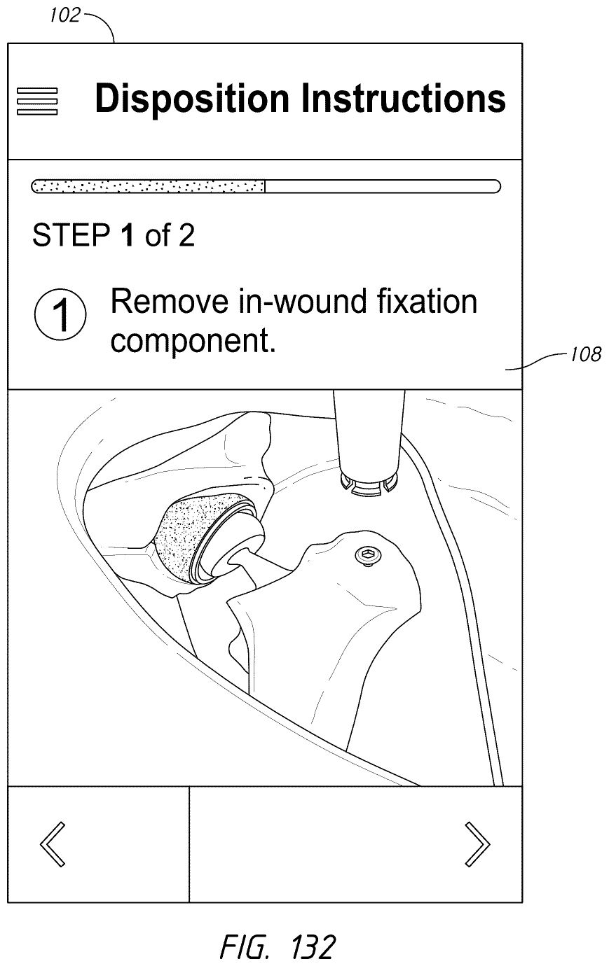

The method can include disposition instructions. The method can include removing the femur registration marker 154. FIG. 132 illustrates the graphical user interface 108 of the navigation device 102 providing directions to the user related to this step.

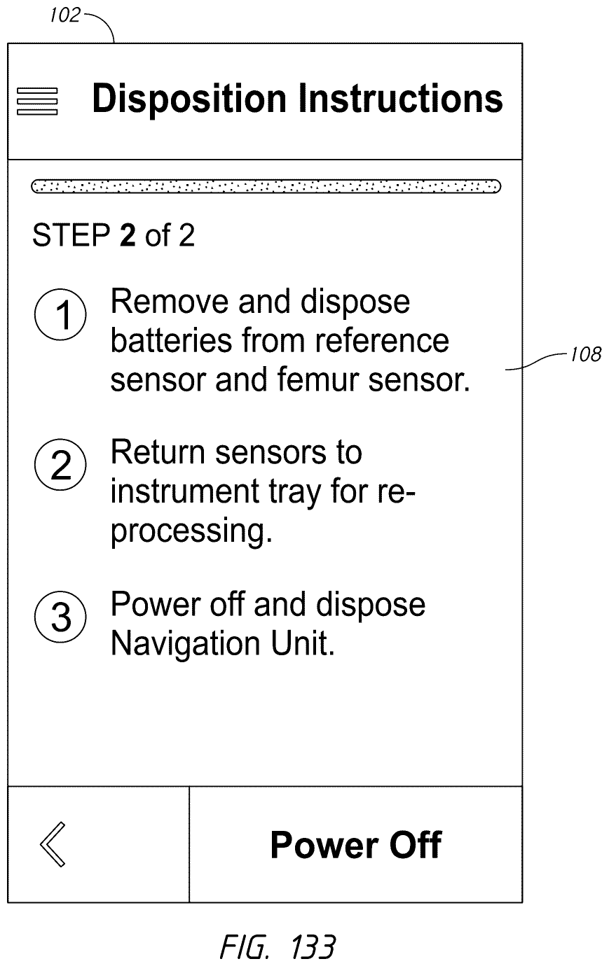

The method can include disposition instructions. The method can include removing the femur sensor battery 164 and the reference sensor battery assembly 166. The method can include returning the reference sensor device 104 and the femur sensor device 106 to the tray base 162. The method can include powering off and disposing the navigation device 102. FIG. 133 illustrates the graphical user interface 108 of the navigation device 102 providing directions to the user related to this step.

Although these inventions have been disclosed in the context of certain preferred embodiments and examples, it will be understood by those skilled in the art that this application extends beyond the specifically disclosed embodiments to other alternative embodiments and/or uses of the invention and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or sub-combinations of the specific features and aspects of the embodiments may be made and still fall within the scope of the application. Accordingly, it should be understood that various features and aspects of the disclosed embodiments can be combined with or substituted for one another in order to form varying modes of the disclosed embodiments. Thus, it is intended that the scope of the present invention herein disclosed should not be limited by the particular disclosed embodiments described above, but should be determined only by a fair reading of the claims that follow.

Similarly, this method of disclosure, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

1. A method of performing a hip joint replacement procedure on a patient, the hip joint comprises a pelvis and a femur that is movable relative to the pelvis, the method comprising:
positioning the femur relative to the pelvis;
registering a point of a femur registration marker;
inserting a cup and a liner into an acetabulum of the pelvis;
inserting a liner registration trial into the liner and thereafter registering a center of rotation; and
registering the point of the femur registration marker after inserting the cup.

2. The method of claim 1, further comprising coupling the liner registration trial to a registration wand.

3. The method of claim 2, wherein registering the center of rotation comprises registering a point on the registration wand.

4. The method of claim 1, further comprising positioning a femur sensor device on a distal thigh of the patient while registering the point of the femur registration marker.

5. The method of claim 1, further comprising positioning a femur sensor device on the greater trochanter while registering the point of the femur registration marker.

6. The method of claim 1, wherein registering the point of the femur registration marker comprises coupling a reference sensor device to a registration probe.

7. The method of claim 1, wherein registering the center of rotation comprises coupling a reference sensor device to a registration probe.

8.-33. (canceled)

34. A system for orthopedic surgery, comprising:
a femur sensor device, wherein the femur sensor device comprises one or more sensors configured to sense changes in orientation and rotation; and
a registration wand, wherein the femur sensor device is configured to couple to the registration wand.

35. The system of claim 34, further comprising a plurality of liner registration trials, each liner registration trial configured to couple to the registration wand.

36. The system of claim 34, further comprising a liner registration trial configured to couple to the registration wand, wherein liner registration trial is configured to be inserted into a liner of a cup.

37. The system of claim 34, wherein the registration wand comprises a divot, wherein a tip of a registration probe is configured to contact the divot to register the registration wand.

38. The system of claim 34, further comprising a femur registration marker.

39. The system of claim 34, wherein the femur sensor device is configured to determine an angle of a femur relative to a pelvis.

40. The method of claim 1, further comprising correcting for changes in orientation by doing a virtual rotation of the femur about the center of rotation.

41. The method of claim 1, further comprising correcting for changes in a femur angle with respect to the pelvis.

42. The method of claim 1, wherein the femur sensor device performs a virtual transformation about the center of rotation.

43. The method of claim 1, further comprising placing the femur relative to the pelvis within a range of positions.

44. The method of claim 1, wherein a thigh plate remains mounted on a distal thigh during cup placement.

45. The method of claim 1, wherein a fixation plate remains mounted on a greater trochanter during cup placement.

46. The method of claim 1, wherein a virtual repositioning facilitates accurate measurements of leg length and/or joint offset.
